(19) United States
(12) Patent Application Publication
(10) Pub. No.: US 2005/0136044 A1
(43) Pub. Date: Jun. 23, 2005

(54) Butyrylcholinesterase variants that alter the activity of chemotherapeutic agents

(76) Inventors: Jeffry D. Watkins, Encinitas, CA (US); James D. Pancook, San Diego, CA (US)

Correspondence Address: CATHRYN CAMPBELL;McDERMOTT, WILL & EMERY, Suite 700, 4370 La Jolla Village Drive, San Diego, CA, 92122, US

(21) Appl. No.: 10/728,723

(22) Filed: Dec. 4, 2003

(51) Int. Cl. A61K 38/46; C07H 21/04; C12P 17/18; C12N 9/18

(52) U.S. Cl. 424/94.6; 435/197; 435/119; 514/283; 536/23.2; 424/178.1; 530/388.26

(57) ABSTRACT

The invention provides a butyrylcholinesterase variant having the amino acid sequence selected from SEQ ID NOS: 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof. In addition, the invention provides a method of converting a camptothecin derivative to a topoisomerase inhibitor by contacting the camptothecin derivative with a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, under conditions that allow conversion of a camptothecin derivative to a topoisomerase inhibitor. Further, the invention provides a method of treating cancer by administering to an individual an effective amount of a butyrylcholinesterase variant selected from SEQ ID NO: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, exhibiting increased capability to convert a camptothecin derivative to a topoisomerase inhibitor compared to butyrylcholinesterase.

This application is based on, and claims the benefit of, U.S. Provisional Application No. 60/______ (yet to be assigned), filed Dec. 4, 2002, which was converted from U.S. Ser. No. 10/310,666, and entitled BUTYRYLCHOLINESTERASE VARIANTS THAT ALTER THE ACTIVITY OF CHEMOTHERAPEUTIC AGENTS, and which is incorporated herein by reference.

### BACKGROUND OF THE INVENTION

This invention relates to butyrylcholinesterase variants and, more specifically to the production and therapeutic use thereof.

Cancer is one of the leading causes of death in the United States. Each year, more than half a million Americans die from cancer, and more than one million are newly diagnosed with the disease. In cancer, neoplastic cells escape from their normal growth regulatory mechanisms and proliferate in an uncontrolled fashion, leading to the development of a malignant tumor. Tumor cells can metastasize to secondary sites if treatment of the primary tumor is either not complete or not initiated before substantial progression of the disease. Early diagnosis and effective treatment of malignant tumors is therefore essential for survival.

The current methods for treating cancer include surgery, radiation therapy and chemotherapy. A major problem with each of these treatments is their lack of specificity for cancer cells and numerous side-effects. For instance, due to their toxicity to normal tissues, the amount of radiation or chemotherapeutic agent that can be safely used is often inadequate to kill all neoplastic cells. Even a few residual neoplastic cells can be lethal, as they can rapidly proliferate and metastasize to other sites. Unfortunately, the toxicity associated with radiation and chemotherapy is manifested by unpleasant side effects, including nausea and hair loss, that severely reduce the quality of life for the cancer patient undergoing these treatments. Clearly, a more selective and effective means of treating cancer is needed.

Recently, classes of chemotherapeutic agents have been discovered which are activated within the body to produce a metabolic product which is toxic to cancer cells. These chemotherapeutic agents are sometimes referred to as “pro-drugs” since they are converted within the body to the active drug. Such chemotherapeutic agents include paxlitaxel prodrug and camptothecin (CPT-11). These agents are metabolized by endogenous carboxylesterases, such as butyrylcholinesterase, to yield active drugs such as paxlitaxel and SN-38, respectively. Unfortunately, although these chemotherapeutic agents have good antitumor activity in vitro, several side effects have been reported with these drugs in patients such as diarrhea, hair loss, nausea, vomiting, and cholinergic symptoms.

The low therapeutic index of these chemotherapeutic agents limits their use for cancer therapy. Because higher doses of these agents result in more side effects, a different method is needed to make these agents more effective. One method is to increase the efficacy of conversion of these agents within the body into the active drug. A number of naturally occurring human butyrylcholinesterases as well as species variations are known, however none of these enzymes exhibits increased pro-drug hydrolysis activity. In addition, enzymes derived from non-human species and intercellular enzymes have been tested for ability to convert pro-drugs into active drugs. However, both enzymes derived from non-human species and intercellular enzymes can be immunogenic which severely limits their use. Advantageously, human butyrylcholinesterase is located in the plasma and is less immunogenic.

Thus, there exists a need for butyrylcholinesterase variants capable of altering the activity of chemotherapeutic agents more efficiently than wild-type butyrylcholinesterase. The present invention satisfies this need and provides related advantages as well.

### SUMMARY OF THE INVENTION

The invention provides a butyrylcholinesterase variant having the amino acid sequence selected from SEQ ID NOS: 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof.

In addition, the invention provides a method of converting a camptothecin derivative to a topoisomerase inhibitor by contacting the camptothecin derivative with a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, under conditions that allow conversion of a camptothecin derivative to a topoisomerase inhibitor.

Further, the invention provides a method of treating cancer by administering to an individual an effective amount of a butyrylcholinesterase variant selected from SEQ ID NO: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, exhibiting increased capability to convert a camptothecin derivative to a topoisomerase inhibitor compared to butyrylcholinesterase.

### BRIEF DESCRIPTION OF THE DRAWINGS

FIG. 1 shows a representative o-nitrophenyl acetate assay showing butyrylcholinesterase variants with increased carboxylesterase activity.

FIG. 2 shows the chemical structure of CPT-11 and SN-38 and the conversion of CPT-11 to SN-38 by carboxylesterase activity.

FIG. 3 shows a high performance liquid chromatography (HPLC) assay for the formation of SN-38. FIG. 3 shows conditioned media from cells that were mock-transfected. The conditioned media was exposed to CPT-11 and analyzed by HPLC for the formation of SN-38.

FIG. 4 shows a high performance liquid chromatography (HPLC) assay for the formation of SN-38 using conditioned media from cells that were transfected with the F227A variant. The conditioned media was exposed to CPT-11 and analyzed by HPLC for the formation of SN-38. The CPT-11 and SN-38 peaks are labeled.

FIG. 5 shows a high performance liquid chromatography (HPLC) assay for the formation of SN-38 using conditioned media from cells that were transfected with the F227A/L286S variant. The conditioned media was exposed to CPT-11 and analyzed by HPLC for the formation of SN-38. The CPT-11 and SN-38 peaks are labeled.

FIG. 6 shows the results of an MTT cytotoxicity assay. CPT-11 was incubated with wild-type butyrylcholinesterase, the 6-6 variant, or F227A/L286Q variant to activate the CPT-11. The percent of SW38 colon carcinoma cells that were killed when exposed to the activated CPT-11 is shown and compared to CPT-11 that was not incubated with butyrylcholinesterase or a butyrylcholinesterase variant (lanes labeled “mock”).

FIG. 7 shows an ELISA assay demonstrating binding of expressed anti-EGFR-BChE L530 to anti-kappa capture antibody and measuring activity of bound butyrylcholinesterase by butyrylthiocholine hydrolysis.

FIG. 8 shows an ELISA assay measuring butyrylcholinesterase enzyme activity of the anti-EGFR-BChE L530 specifically bound to a cell membrane preparation containing the EGFR antigen.

FIG. 9 shows the nucleotide and amino acid sequence of the mouse anti-EGFR variable light chain (SEQ ID NOS: 17 and 18).

FIG. 10 shows the nucleotide and amino acid sequence of the mouse anti-EGFR variable heavy chain and constant heavy chain hinge region of L530 (SEQ ID NOS: 19 and 20).

FIG. 11 shows the nucleotide and amino acid sequence of human butyrylcholinesterase (SEQ ID NOS: 21 and 22). The positions of F227, T284, L286, and S287 are marked in bold and underlined.

FIG. 12 shows a table that depicts quantitation of SN38 conversion by butyrylcholinesterase variant 4-1.

FIG. 13 shows a Hofstee plot of CPT-11 hydrolysis by butyrylcholinesterase variant 4-1.

FIG. 14 shows binding of antibody-butyrylcholinesterase fusion proteins to fixed SKW tumor cells.

FIG. 15 shows targeted cytotoxocity against SKW tumor cells of an anti-CD20-butyrylcholinesterase fusion protein.

FIG. 16 shows a table setting forth structural and functional characteristics of the butyrylcholinesterase variant F227A (SEQ ID NO:2).

FIG. 17 shows a table setting forth structural and functional characteristics of the butyrylcholinesterase variants designated SEQ ID NOS: 24 through 176, all of which are double mutations that include the F227A change as one of the two amino acid changes.

FIG. 18 shows a table setting forth structural and functional characteristics of the butyrylcholinesterase variants designated SEQ ID NOS: 178 through 196, all of which include the F227A change as one of the amino acid changes.

FIG. 19 shows the amino acid sequence (SEQ ID NO: 202) and corresponding nucleotide sequence (SEQ ID NO: 201) of an anti-CD20 VH-CH1 hinge cys L530 BChE.4-1 heavy chain construct. This is the fusion protein heavy chain comprised of the anti-CD20 antibody variable heavy chain region with a cysteine-containing hinge region and the L530 fragment (SEQ ID NO: 204) of the butyrylcholinesterase variant designated SEQ ID NO: 180, which incorporates the 4-1 variant amino acids (H77F/F227A/P285N/V331A).

FIG. 20 shows the amino acid sequence (SEQ ID NO: 198) and corresponding nucleotide sequence (SEQ ID NO: 197) of an anti-CD20 light chain.

### DETAILED DESCRIPTION OF THE INVENTION

This invention provides butyrylcholinesterase variants that exhibit increased ability to convert chemotherapeutic agent pro-drugs into active drugs. The identification of butyrylcholinesterase variants that exhibit increased ability to convert chemotherapeutic agent pro-drugs into active drugs provides treatment options for cancer.

In one embodiment, the invention provides a method of treating an individual suffering from symptoms of cancer. The butyrylcholinesterase variants of the invention hold significant clinical value because of their capability to convert pro-drugs to active drugs at a higher rate than any of the known naturally occurring wild-type butyrylcholinesterase. It is this increase in pro-drug conversion activity that enables a more effective treatment for cancer with less side effect which sets the butyrylcholinesterase variants of the invention apart from other treatment options.

In one embodiment, the invention provides a method of converting a camptothecin derivative to a topoisomerase inhibitor by contacting the camptothecin derivative with a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, or functional fragment thereof, under conditions that allow conversion of a camptothecin derivative to a topoisomerase inhibitor.

As used herein, the term “butyrylcholinesterase” is intended to refer a polypeptide having the sequence of naturally occurring butyrylcholinesterase. A naturally occurring butyrylcholinesterase can be of any species origin, for example, human, primate, horse, or murine. Therefore, a butyrylcholinesterase can be, for example a vertebrate or invertebrate butyrylcholinesterase, for example a mammalian butyrylcholinesterase. In addition, a butyrylcholinesterase of the invention can be a polymorphism or any other allelic variation of a naturally occurring butyrylcholinesterase. A nucleic acid encoding a butyrylcholinesterase of the invention encodes a polypeptide having the sequence of any naturally occurring butyrylcholinesterase. Therefore, a nucleic acid encoding a butyrylcholinesterase can encode a butyrylcholinesterase of any species origin, for example, human, primate, horse, or murine. In addition, a nucleic acid encoding a butyrylcholinesterase encompasses any naturally occurring allele or polymorphism. A GenBank accession number for human butyrylcholinesterase is M16541.

As used herein, the term “butyrylcholinesterase variant” is intended to refer to a molecule that is structurally similar to butyrylcholinesterase, but differs by at least one amino acid from butyrylcholinesterase. A butyrylcholinesterase variant has the amino acid sequence as butyrylcholinesterase and exhibits enhanced metabolic capability to convert a camptothecin derivative to a topoisomerase inhibitor. In this regard, a butyrylcholinesterase variant can possess, for example, reduced, the or increased capability to convert a camptothecin derivative to a topoisomerase inhibitor compared to butyrylcholinesterase. For example, the conversion capability of a butyrylcholinesterase variant of the invention can be increased by a factor of 2, 5, 10, 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 4000, 5000, or more.

A butyrylcholinesterase variant can have a single amino acid alteration as well as multiple amino acid alterations compared to buyrylcholinesterase. A specific example of a butyrylcholinesterase variant is butyrylcholinesterase having the amino acid alanine at position 227, of which the amino acid sequence and encoding nucleic acid sequence is designated as SEQ ID NOS: 2 and 1, respectively. A further example is the butyrylcholinesterase variant the amino acid Phenylalanine at position 77, the amino acid alanine at position 227, the amino acid Asparagine at position 285, the amino acid alanine at position 331, of which the amino acid sequence and encoding nucleic acid sequence is designated as SEQ ID NOS: 180 and 179, respectively, and which exhibits at least a three thousand-fold increased capability to convert the camptothecin derivative CPT-11 to the topoisomerase inhibitor SN-38 compared to butyrylcholinesterase. The term is also intended to include butyrylcholinesterase variants encompassing, for example, modified forms of naturally occurring amino acids such as D-stereoisomers, non-naturally occurring amino acids, amino acid analogues and mimetics so long as such variants have substantially the same amino acid sequence as butyrylcholinesterase and exhibit capability to convert a camptothecin derivative to a topoisomerase inhibitor. A butyrylcholinesterase variant of the invention can have one or amino acid alterations outside of the regions determined or predicted to be important for conversion capability of a camptothecin derviative to a topoisomerase inhibitor herein. Furthermore, a butyrylcholinesterase variant of the invention can have one or more additional modifications that do not significantly change its capability to convert a camptothecin derivative to a topoisomerase inhibitor activity. A butyrylcholinesterase variant of the invention can also have increased stability compared to butyrylcholinesterase.

As used herein, the butyrylcolinesterase variants of the invention include sequences that are substantially the same as the reference amino acid sequence and a butyrylcholinesterase variant is thus intended to include a polypeptide, fragment or segment having an identical amino acid sequence, or a polypeptide, fragment or segment having a similar, non-identical sequence that is considered by those skilled in the art to be a functionally equivalent amino acid sequence. An amino acid sequence that is substantially identical to a reference butyrylcholinesterase or butyrylcholinesterase variant of the invention can have at least 70%, at least 80%, at least 81%, at least 83%, at least 85%, at least 90%, at least 95% or more identity to the reference butyrylcholinesterase. Substantially the same amino acid sequence is also intended to include polypeptides encompassing, for example, modified forms of naturally occurring amino acids such as D-stereoisomers, non-naturally occurring amino acids, amino acid analogues and mimetics so long as such polypeptides retain functional activity as defined above. A biological activity of a butyrylcholinesterase variant of the invention is the capability to convert a camptothecin derivative to a topoisomerase inhibitor, as described herein. For example, the butyrylcholinesterase variant F227A designated SEQ ID NO: 2 exhibits at least a three-fold increased capability to convert the camptothecin derivative CPT-11 to the topoisomerase inhibitor SN-38 compared to butyrylcholinesterase. A further example is the butyrylcholinesterase variant H77F, F227A, P285N, V331A designated SEQ ID NO: 180, which exhibits at least a three thousand-fold increased capability to convert the camptothecin derivative CPT-11 to the topoisomerase inhibitor SN-38 compared to butyrylcholinesterase.

It is understood that minor modifications in the primary amino acid sequence can result in a polypeptide that has a substantially equivalent function as compared to a polypeptide of the invention. These modifications can be deliberate, as through site-directed mutagenesis, or may be accidental such as through spontaneous mutation. For example, it is understood that only a portion of the entire primary structure of a butyrylcholinesterase variant can be required in order to effect the capability to convert a camptothecin derivative to a topoisomerase inhibitor. Moreover, fragments of the sequence of a butyrylcholinesterase variant of the invention are similarly included within the definition as long as at least one biological function of the butyrylcholinesterase variant is retained. It is understood that various molecules can be attached to a polypeptide of the invention, for example, other polypeptides, carbohydrates, lipids, or chemical moieties.

The nucleic acid molecules of the invention include nucleic acid sequences that are substantially the same to the reference nucleic acid molecule of the invention or a fragment thereof and are intended to include sequences having one or more additions, deletions or substitutions with respect to the reference sequence, so long as the nucleic acid molecule retains its ability to selectively hybridize with the subject nucleic acid molecule under moderately stringent conditions, or highly stringent conditions. Moderately stringent conditions are intended to include hybridization conditions equivalent to hybridization of filter-bound nucleic acid in 50% formamide, 5× Denhart's solution, 5×SSPE, 0.2% SDS at 42° C., followed by washing in 0.2×SSPE, 0.2% SDS, at 50°. As used herein, highly stringent conditions are conditions equivalent to hybridization of filter-bound nucleic acid in 50% formamide, 5× Denhart's solution, 5×SSPE, 0.2% SDS at 42° C., followed by washing in 0.2×SSPE, 0.2% SDS, at 65°. Other suitable moderately stringent and highly stringent hybridization buffers and conditions are well known to those of skill in the art and are described, for example, in Sambrook et al., Molecular Cloning: A Laboratory Manual, Cold Spring Harbor Laboratory, New York (1992) and in Ansubel et al., Current Protocols in Molecular Biology, John Wiley and Sons, Baltimore, Md. (1998). Thus, it is not necessary that two nucleic acids exhibit sequence identity to be substantially complimentary, only that they can specifically hybridize or be made to specifically hybridize without detectible cross reactivity with other similar sequences.

In general, a nucleic acid molecule that has substantially the same nucleotide sequence as a reference sequence will have greater than about 60% identity, such as greater than about 65%, 70%, 75% identity with the reference sequence, such as greater than about 80%, 85%, 90%, 95%, 97% or 99% identity to the reference sequence over the length of the two sequences being compared. Identity of any two nucleic acid sequences can be determined by those skilled in the art based, for example, on a BLAST 2.0 computer alignment, using default parameters. BLAST 2.0 searching is available at ncbi.nlm.nih.gov/gorf/bl2.html., as described by Tatiana et al., FEMS Microbiol Lett. 174: 247-250 (1999).

As used herein, the term “fragment” when used in reference to a nucleic acid encoding the claimed polypeptides is intended to mean a nucleic acid having substantially the same sequence as a portion of a nucleic acid encoding a polypeptide of the invention or segments thereof. The nucleic acid fragment is sufficient in length and sequence to selectively hybridize to a butyrylcholinesterase variant encoding nucleic acid or a nucleotide sequence that is complimentary to a butyrylcholinesterase variant encoding nucleic acid. Therefore, fragment is intended to include primers for sequencing and polymerase chain reaction (PCR) as well as probes for nucleic acid blot or solution hybridization.

Similarly, the term “functional fragment” when used in reference to a nucleic acid encoding a butyrylcholinesterase or butyrylcholinesterase variant is intended to refer to a portion of the nucleic acid that encodes a portion of the butyrylcholinesterase or butyrylcholinesterase variant that still retains some or all of the metabolic conversion capability of the parent polypeptide. A functional fragment of a polypeptide of the invention exhibiting a functional activity can have, for example, at least 6 contiguous amino acid residues from the polypeptide, at least 8, 10, 15, 20, 30 or 40 amino acids, and often has at least 50, 75, 100, 200, 300, 400 or more amino acids of a polypeptide of the invention, up to the full length polypeptide minus one amino acid. An example of a functional fragment of a butyrylcholinesterase variant of the invention is a variant that is truncated at position 530, which is a Leucine residue in the wild-type butytlcholinesterase. While a L530 truncation has no effect on the functional activity of the corresponding full-length variant, the truncation prevents formation of tetramers, thereby enhancing bioactivity and pharmacokinetic properties of the variant. Therefore, a butyrylcholinestrase variant of the invention includes an L530 truncation, which is considered a functional fragment of the reference variant.

As used herein, the term “functional fragment” in regard to a polypeptide of the invention, refers to a portion of the reference polypeptide that is capable of exhibiting or carrying out a functional activity of the reference polypeptide. A functional fragment of a polypeptide of the invention exhibiting a functional activity can have, for example, at least 6 contiguous amino acid residues from the polypeptide, at least 8, 10, 15, 20, 30 or 40 amino acids, and often has at least 50, 75, 100, 200, 300, 400 or more amino acids of a polypeptide of the invention, up to the full length polypeptide minus one amino acid. The appropriate length and amino acid sequence of a functional fragment of a polypeptide of the invention can be determined by those skilled in the art, depending on the intended use of the functional fragment. For example, a functional fragment of a butyrylcholinesterase or butyrylcholinesterase variant is intended to refer to a portion of the butyrylcholinesterase or butyrylcholinesterase variant that still retains some or all of the metabolic conversion capability of the parent polypeptide.

As used herein, the term “antibody” is intended to mean a polypeptide produced in response to an antigen which has the ability to specifically bind to the antigen which induced its formation. Antibodies include, for example, monoclonal and polyclonal antibodies, single chain antibodies, chimeric antibodies, bifunctional or bispecific antibodies, humanized antibodies, human antibodies, and complementary determining region (CDR)-grafted antibodies, including compounds which include CDR or antigen-binding sequences, which specifically bind to a polypeptide of the invention. An “antibody fragment” refers to a portion of an antibody polypeptide that retains some part of the function of the intact antibody. For example, an antibody fragment can retain some or all of the antigen binding ability of the intact antibody. Antibody fragments include, for example, Fab, Fab′, F(ab′)₂, and Fv. Screening assays to determine binding specificity or exclusivity of an antibody or antibody fragment of the invention are well known in the art (see Harlow et al. (Eds), Antibodies A Laboratory Manual; Cold Spring Harbor Laboratory; Cold Spring Harbor, N.Y. (1988)).

Antibodies that can be used in the invention can be produced using any method well known in the art, using any polypeptide, or immunogenic fragment thereof, of the invention. For example, a humanized antibody can be prepared with fully human germline framework regions. In addition, immunogenic polypeptides can be isolated from natural sources, from recombinant host cells, or can be chemically synthesized. Methods for synthesizing such peptides are known in the art, for example, as in R. P. Merrifield, J. Amer. Chem. Soc. 85: 2149-2154 (1963); J. L. Krstenansky, et al., FEBS Lett. 211: 10 (1987).

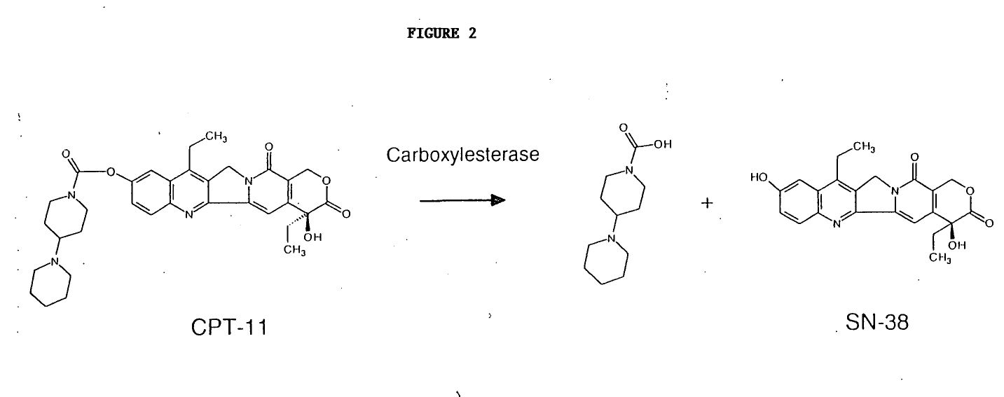

As used herein, the term “camptothecin derivative” refers to a compound that has a structure the same or substantially the same as camptothecin and that can be hydrolyzed by butyrylcholinesterase or a butyrylcholinesterase variant. For example, a camptothecin derivative can be hydrolyzed by the F227A/L286Q variant (SEQ ID NO:6). Camptothecin is derived from the stem bark of a Chinese tree called Camptotheca acuminata Decaisne. Camptothecin derivatives can inhibit DNA topoisomerase I through their metabolic break-down products. The structure of a water soluble campthothecin derivative, CPT-11, is shown in FIG. 2. The chemical name of CPT-11 is 7-ethyl-10-[4-(1-piperidino)-1-piperidine]carbonyloxycamptothecin. CPT-11 is also known by the names CAMPTOSAR and Irinotecan. Members of the camptothecins include, for example, topotecan, irinotecan, 9-aminocamptothecin and 9-nitrocamptothecin which are analogs of the plant alkaloid 20(S)-camptothecin.

As used herein, the term “topoisomerase inhibitor” refers to a compound that can inhibit a topoisomerase. Several topoisomerases are known in the literature. For example, a topoisomerase inhibitor can inhibit a type I topoisomerase, such as topoisomerase I, or a type II topoisomerase. Type I enzymes act by making a transient break in one strand of DNA and type II enzymes act by introducing a transient double strand break. Some DNA topoisonerases can relax or remove only negative supercoils from DNA while others can relax both negative and positive supercoils and still others can introduce negative supercoils. An example of a topoisomerase inhibitor is SN-38, the structure of which is shown in FIG. 2.

The chemical name of SN-38 is 7-ethyl-10-hydroxycamptothecin. In vitro, SN38 has been shown to be greater than 1000-fold more cytotoxic than CPT-11 (Pavillard et al., Cancer Chemother Pharmacol. 49: 329-35(2002)). In humans it is believed that prodrug conversion takes place primarily in the liver through the activities of two carboxylesterase isoforms, human carboxylesterase-1 (hCE-1) and human carboxylesterase-2 (hCE-2) (Humerickhouse et al., Cancer Res 60: 1189-92(2000)). The Km values are 3.4 μM and 43 μM for hCE-2 and hCE-1, respectively, and the catalytic efficiency of hCE-2 is 60-fold higher than that of hCE-1. At pharmacologically relevant concentrations of the drug (˜1-10 μM), hCE-2 converts CPT-11 to SN38 at a 25-30-fold higher rate than hCE-1 (12). SN-38 can interact with topoisomerase I and DNA to form cleavage complexes, and prevent resealing of the topoisomerase I-mediated DNA single strand breaks. This interaction eventually can lead to double-strand DNA breaks and cell death such as apoptosis.

As used herein, the term “campthothecin conversion activity” or campthothecin hydrolysis activity is intended to mean the chemical conversion of a campthothecin derivative to a topoisomerase inhibitor. For example, the conversion of CPT-11 to SN-38 is shown in FIG. 2. Conversion activity can be measured both directly or indirectly using several assays described herein (see Example II, III, and IV).

As used herein, the term “effective amount” is intended to mean an amount of a butyrylcholinesterase variant of the invention that can reduce the severity of cancer. Reduction in severity includes, for example, an arrest or a decrease in symptoms, physiological indicators, biochemical markers or metabolic indicators. Symptoms of cancer include, for example, weight loss, pain, and organ failure. As used herein, the term “treating” is intended to mean causing a reduction in the severity of cancer.

The invention provides a butyrylcholinesterase variant having the amino acid sequence selected from SEQ ID NOS: 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof. The invention also provides a butyrylcholinesterase variant where the amino acid contains SEQ ID NO: 4, or functional fragment thereof. The invention further provides a butyrylcholinesterase variant where the amino acid contains SEQ ID NO: 6, or functional fragment thereof. The invention also provides a butyrylcholinesterase variant where the amino acid contains SEQ ID NO: 8, or functional fragment thereof. The invention further provides a butyrylcholinesterase variant where the amino acid contains SEQ ID NO: 10, or functional fragment thereof. The invention also provides a butyrylcholinesterase variant where the amino acid contains SEQ ID NO: 12, or functional fragment thereof. The invention further provides a butyrylcholinesterase variant where the amino acid contains SEQ ID NO: 14, or functional fragment thereof. The invention further provides a butyrylcholinesterase variant where the amino acid contains SEQ ID NO: 24, or functional fragment thereof. The invention further provides a butyrylcholinesterase variant where the amino acid contains SEQ ID NO: 26, or functional fragment thereof. The invention further provides a butyrylcholinesterase variant where the amino acid contains SEQ ID NO: 28, or functional fragment thereof. The invention further provides a butyrylcholinesterase variant where the amino acid contains SEQ ID NO: 30, or functional fragment thereof. The invention further provides a butyrylcholinesterase variant where the amino acid contains SEQ ID NO: 32, or functional fragment thereof.

The invention further provides a butyrylcholinesterase variant where the amino acid contains SEQ ID NO: 34, or functional fragment thereof. The invention further provides a butyrylcholinesterase variant where the amino acid contains SEQ ID NO: 36, or functional fragment thereof. The invention further provides a butyrylcholinesterase variant where the amino acid contains SEQ ID NO: 38, or functional fragment thereof. The invention further provides a butyrylcholinesterase variant where the amino acid contains SEQ ID NO: 40, or functional fragment thereof. The invention further provides a butyrylcholinesterase variant where the amino acid contains SEQ ID NO: 42, or functional fragment thereof. The invention further provides a butyrylcholinesterase variant where the amino acid contains SEQ ID NO: 44, or functional fragment thereof. The invention further provides a butyrylcholinesterase variant where the amino acid contains SEQ ID NO: 46, or functional fragment thereof. The invention further provides a butyrylcholinesterase variant where the amino acid contains SEQ ID NO: 48, or functional fragment thereof. The invention further provides a butyrylcholinesterase variant where the amino acid contains SEQ ID NO: 50, or functional fragment thereof. The invention further provides a butyrylcholinesterase variant where the amino acid contains SEQ ID NO: 52, or functional fragment thereof. The invention further provides a butyrylcholinesterase variant where the amino acid contains SEQ ID NO: 54, or functional fragment thereof. The invention further provides a butyrylcholinesterase variant where the amino acid contains SEQ ID NO: 56, or functional fragment thereof. The invention further provides a butyrylcholinesterase variant where the amino acid contains SEQ ID NO: 58, or functional fragment thereof. The invention further provides a butyrylcholinesterase variant where the amino acid contains SEQ ID NO: 60, or functional fragment thereof. The invention further provides a butyrylcholinesterase variant where the amino acid contains SEQ ID NO: 62, or functional fragment thereof. The invention further provides a butyrylcholinesterase variant where the amino acid contains SEQ ID NO: 64, or functional fragment thereof. The invention further provides a butyrylcholinesterase variant where the amino acid contains SEQ ID NO: 66, or functional fragment thereof. The invention further provides a butyrylcholinesterase variant where the amino acid contains SEQ ID NO: 68, or functional fragment thereof. The invention further provides a butyrylcholinesterase variant where the amino acid contains SEQ ID NO: 70, or functional fragment thereof. The invention further provides a butyrylcholinesterase variant where the amino acid contains SEQ ID NO: 72, or functional fragment thereof. The invention further provides a butyrylcholinesterase variant where the amino acid contains SEQ ID NO: 74, or functional fragment thereof. The invention further provides a butyrylcholinesterase variant where the amino acid contains SEQ ID NO: 76, or functional fragment thereof. The invention further provides a butyrylcholinesterase variant where the amino acid contains SEQ ID NO: 78, or functional fragment thereof. The invention further provides a butyrylcholinesterase variant where the amino acid contains SEQ ID NO: 80, or functional fragment thereof. The invention further provides a butyrylcholinesterase variant where the amino acid contains SEQ ID NO: 82, or functional fragment thereof. The invention further provides a butyrylcholinesterase variant where the amino acid contains SEQ ID NO: 84, or functional fragment thereof. The invention further provides a butyrylcholinesterase variant where the amino acid contains SEQ ID NO: 86, or functional fragment thereof. The invention further provides a butyrylcholinesterase variant where the amino acid contains SEQ ID NO: 88, or functional fragment thereof. The invention further provides a butyrylcholinesterase variant where the amino acid contains SEQ ID NO: 90, or functional fragment thereof. The invention further provides a butyrylcholinesterase variant where the amino acid contains SEQ ID NO: 92, or functional fragment thereof. The invention further provides a butyrylcholinesterase variant where the amino acid contains SEQ ID NO: 94, or functional fragment thereof. The invention further provides a butyrylcholinesterase variant where the amino acid contains SEQ ID NO: 96, or functional fragment thereof. The invention further provides a butyrylcholinesterase variant where the amino acid contains SEQ ID NO: 98, or functional fragment thereof. The invention further provides a butyrylcholinesterase variant where the amino acid contains SEQ ID NO: 100, or functional fragment thereof. The invention further provides a butyrylcholinesterase variant where the amino acid contains SEQ ID NO: 102, or functional fragment thereof. The invention further provides a butyrylcholinesterase variant where the amino acid contains SEQ ID NO: 104, or functional fragment thereof. The invention further provides a butyrylcholinesterase variant where the amino acid contains SEQ ID NO: 106, or functional fragment thereof. The invention further provides a butyrylcholinesterase variant where the amino acid contains SEQ ID NO: 108, or functional fragment thereof. The invention further provides a butyrylcholinesterase variant where the amino acid contains SEQ ID NO: 110, or functional fragment thereof. The invention further provides a butyrylcholinesterase variant where the amino acid contains SEQ ID NO: 112, or functional fragment thereof. The invention further provides a butyrylcholinesterase variant where the amino acid contains SEQ ID NO: 114, or functional fragment thereof. The invention further provides a butyrylcholinesterase variant where the amino acid contains SEQ ID NO: 116, or functional fragment thereof. The invention further provides a butyrylcholinesterase variant where the amino acid contains SEQ ID NO: 118, or functional fragment thereof. The invention further provides a butyrylcholinesterase variant where the amino acid contains SEQ ID NO: 120, or functional fragment thereof. The invention further provides a butyrylcholinesterase variant where the amino acid contains SEQ ID NO: 122, or functional fragment thereof. The invention further provides a butyrylcholinesterase variant where the amino acid contains SEQ ID NO: 124, or functional fragment thereof. The invention further provides a butyrylcholinesterase variant where the amino acid contains SEQ ID NO: 126, or functional fragment thereof. The invention further provides a butyrylcholinesterase variant where the amino acid contains SEQ ID NO: 128, or functional fragment thereof. The invention further provides a butyrylcholinesterase variant where the amino acid contains SEQ ID NO: 130, or functional fragment thereof. The invention further provides a butyrylcholinesterase variant where the amino acid contains SEQ ID NO: 132, or functional fragment thereof. The invention further provides a butyrylcholinesterase variant where the amino acid contains SEQ ID NO: 134, or functional fragment thereof. The invention further provides a butyrylcholinesterase variant where the amino acid contains SEQ ID NO: 136, or functional fragment thereof. The invention further provides a butyrylcholinesterase variant where the amino acid contains SEQ ID NO: 138, or functional fragment thereof. The invention further provides a butyrylcholinesterase variant where the amino acid contains SEQ ID NO: 140, or functional fragment thereof. The invention further provides a butyrylcholinesterase variant where the amino acid contains SEQ ID NO: 142, or functional fragment thereof. The invention further provides a butyrylcholinesterase variant where the amino acid contains SEQ ID NO: 144, or functional fragment thereof. The invention further provides a butyrylcholinesterase variant where the amino acid contains SEQ ID NO: 146, or functional fragment thereof. The invention further provides a butyrylcholinesterase variant where the amino acid contains SEQ ID NO: 148, or functional fragment thereof. The invention further provides a butyrylcholinesterase variant where the amino acid contains SEQ ID NO: 150, or functional fragment thereof. The invention further provides a butyrylcholinesterase variant where the amino acid contains SEQ ID NO: 152, or functional fragment thereof. The invention further provides a butyrylcholinesterase variant where the amino acid contains SEQ ID NO: 154, or functional fragment thereof. The invention further provides a butyrylcholinesterase variant where the amino acid contains SEQ ID NO: 156, or functional fragment thereof. The invention further provides a butyrylcholinesterase variant where the amino acid contains SEQ ID NO: 158, or functional fragment thereof. The invention further provides a butyrylcholinesterase variant where the amino acid contains SEQ ID NO: 160, or functional fragment thereof. The invention further provides a butyrylcholinesterase variant where the amino acid contains SEQ ID NO: 162, or functional fragment thereof. The invention further provides a butyrylcholinesterase variant where the amino acid contains SEQ ID NO: 164, or functional fragment thereof. The invention further provides a butyrylcholinesterase variant where the amino acid contains SEQ ID NO: 166, or functional fragment thereof. The invention further provides a butyrylcholinesterase variant where the amino acid contains SEQ ID NO: 168, or functional fragment thereof. The invention further provides a butyrylcholinesterase variant where the amino acid contains SEQ ID NO: 170, or functional fragment thereof. The invention further provides a butyrylcholinesterase variant where the amino acid contains SEQ ID NO: 172, or functional fragment thereof. The invention further provides a butyrylcholinesterase variant where the amino acid contains SEQ ID NO: 174, or functional fragment thereof. The invention further provides a butyrylcholinesterase variant where the amino acid contains SEQ ID NO: 176, or functional fragment thereof. The invention further provides a butyrylcholinesterase variant where the amino acid contains SEQ ID NO: 178, or functional fragment thereof. The invention further provides a butyrylcholinesterase variant where the amino acid contains SEQ ID NO: 180, or functional fragment thereof. The invention further provides a butyrylcholinesterase variant where the amino acid contains SEQ ID NO: 182, or functional fragment thereof. The invention further provides a butyrylcholinesterase variant where the amino acid contains SEQ ID NO: 184, or functional fragment thereof. The invention further provides a butyrylcholinesterase variant where the amino acid contains SEQ ID NO: 186, or functional fragment thereof. The invention further provides a butyrylcholinesterase variant where the amino acid contains SEQ ID NO: 188, or functional fragment thereof. The invention further provides a butyrylcholinesterase variant where the amino acid contains SEQ ID NO: 190, or functional fragment thereof. The invention further provides a butyrylcholinesterase variant where the amino acid contains SEQ ID NO: 192, or functional fragment thereof. The invention further provides a butyrylcholinesterase variant where the amino acid contains SEQ ID NO: 194, or functional fragment thereof. The invention further provides a butyrylcholinesterase variant where the amino acid contains SEQ ID NO: 196, or functional fragment thereof.

The invention provides a butyrylcholinesterase variant having a 3000-fold increase in camptothecin conversion activity compared to butyrylcholinesterase, or functional fragment thereof. The invention also provides a butyrylcholinesterase variant having at least a 4-fold, 6-fold, 8-fold, 10-fold, 15-fold, 20-fold, 25-fold, 30-fold, 35-fold, 40-fold, 45-fold, 50-fold, 100-fold, 200-fold, 300-fold, 400-fold, 500-fold, 600-fold, 700-fold, 800-fold, 900-fold, 1000-fold, 1100-fold, 1200-fold, 1300-fold, 1400-fold, 1500-fold, 1600-fold, 1700-fold, 1800-fold, 1900-fold, 2000-fold, 2100-fold, 2200-fold, 2300-fold, 2400-fold, 2500-fold, 2600-fold, 2700-fold, 2800-fold, 2900-fold, 3000-fold, 3100-fold, 3200-fold, 3500-fold or greater increase in camptothecin conversion activity compared to butyrylcholinesterase, or functional fragment thereof.

The invention further provides a nucleic acid encoding a butyrylcholinesterase variant having the nucleic acid sequence selected from SEQ ID NOS: 3, 5, 7, 9, 11, 13, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, and 195, or fragment thereof. In addition the invention provides a nucleic acid encoding a butyrylcholinesterase variant having the amino acid sequence selected from SEQ ID NOS: 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or a functional fragment thereof. Further, the invention provides a nucleic acid encoding a butyrylcholinesterase variant containing the nucleic acid sequence SEQ ID NO: 3, or a functional fragment thereof. The invention also provides a nucleic acid encoding a butyrylcholinesterase variant containing the nucleic acid sequence SEQ ID NO: 5, or a functional fragment thereof. In addition the invention provides a nucleic acid encoding a butyrylcholinesterase variant containing the nucleic acid sequence SEQ ID NO: 7, or a functional fragment thereof. The invention also provides a nucleic acid encoding a butyrylcholinesterase variant containing the nucleic acid sequence SEQ ID NO: 9, or a functional fragment thereof. The invention also provides a nucleic acid encoding a butyrylcholinesterase variant containing the nucleic acid sequence SEQ ID NO: 11, or a functional fragment thereof. The invention further provides a nucleic acid encoding a butyrylcholinesterase variant containing the nucleic acid sequence SEQ ID NO: 13, or a functional fragment thereof. The invention further provides a nucleic acid encoding a butyrylcholinesterase variant containing the nucleic acid sequence SEQ ID NO: 23, or a functional fragment thereof. The invention further provides a nucleic acid encoding a butyrylcholinesterase variant containing the nucleic acid sequence SEQ ID NO: 25, or a functional fragment thereof. The invention further provides a nucleic acid encoding a butyrylcholinesterase variant containing the nucleic acid sequence SEQ ID NO: 27, or a functional fragment thereof. The invention further provides a nucleic acid encoding a butyrylcholinesterase variant containing the nucleic acid sequence SEQ ID NO: 29, or a functional fragment thereof. The invention further provides a nucleic acid encoding a butyrylcholinesterase variant containing the nucleic acid sequence SEQ ID NO: 31, or a functional fragment thereof. The invention further provides a nucleic acid encoding a butyrylcholinesterase variant containing the nucleic acid sequence SEQ ID NO: 33, or a functional fragment thereof. The invention further provides a nucleic acid encoding a butyrylcholinesterase variant containing the nucleic acid sequence SEQ ID NO: 35, or a functional fragment thereof. The invention further provides a nucleic acid encoding a butyrylcholinesterase variant containing the nucleic acid sequence SEQ ID NO: 37, or a functional fragment thereof. The invention further provides a nucleic acid encoding a butyrylcholinesterase variant containing the nucleic acid sequence SEQ ID NO: 39, or a functional fragment thereof. The invention further provides a nucleic acid encoding a butyrylcholinesterase variant containing the nucleic acid sequence SEQ ID NO: 41, or a functional fragment thereof. The invention further provides a nucleic acid encoding a butyrylcholinesterase variant containing the nucleic acid sequence SEQ ID NO: 43, or a functional fragment thereof. The invention further provides a nucleic acid encoding a butyrylcholinesterase variant containing the nucleic acid sequence SEQ ID NO: 45, or a functional fragment thereof. The invention further provides a nucleic acid encoding a butyrylcholinesterase variant containing the nucleic acid sequence SEQ ID NO: 47, or a functional fragment thereof. The invention further provides a nucleic acid encoding a butyrylcholinesterase variant containing the nucleic acid sequence SEQ ID NO: 49, or a functional fragment thereof. The invention further provides a nucleic acid encoding a butyrylcholinesterase variant containing the nucleic acid sequence SEQ ID NO: 51, or a functional fragment thereof. The invention further provides a nucleic acid encoding a butyrylcholinesterase variant containing the nucleic acid sequence SEQ ID NO: 53, or a functional fragment thereof. The invention further provides a nucleic acid encoding a butyrylcholinesterase variant containing the nucleic acid sequence SEQ ID NO: 55, or a functional fragment thereof. The invention further provides a nucleic acid encoding a butyrylcholinesterase variant containing the nucleic acid sequence SEQ ID NO: 57, or a functional fragment thereof. The invention further provides a nucleic acid encoding a butyrylcholinesterase variant containing the nucleic acid sequence SEQ ID NO: 59, or a functional fragment thereof. The invention further provides a nucleic acid encoding a butyrylcholinesterase variant containing the nucleic acid sequence SEQ ID NO: 61, or a functional fragment thereof. The invention further provides a nucleic acid encoding a butyrylcholinesterase variant containing the nucleic acid sequence SEQ ID NO: 63, or a functional fragment thereof. The invention further provides a nucleic acid encoding a butyrylcholinesterase variant containing the nucleic acid sequence SEQ ID NO: 65, or a functional fragment thereof. The invention further provides a nucleic acid encoding a butyrylcholinesterase variant containing the nucleic acid sequence SEQ ID NO: 67, or a functional fragment thereof. The invention further provides a nucleic acid encoding a butyrylcholinesterase variant containing the nucleic acid sequence SEQ ID NO: 69, or a functional fragment thereof. The invention further provides a nucleic acid encoding a butyrylcholinesterase variant containing the nucleic acid sequence SEQ ID NO: 71, or a functional fragment thereof. The invention further provides a nucleic acid encoding a butyrylcholinesterase variant containing the nucleic acid sequence SEQ ID NO: 73, or a functional fragment thereof. The invention further provides a nucleic acid encoding a butyrylcholinesterase variant containing the nucleic acid sequence SEQ ID NO: 75, or a functional fragment thereof. The invention further provides a nucleic acid encoding a butyrylcholinesterase variant containing the nucleic acid sequence SEQ ID NO: 77, or a functional fragment thereof. The invention further provides a nucleic acid encoding a butyrylcholinesterase variant containing the nucleic acid sequence SEQ ID NO: 79, or a functional fragment thereof. The invention further provides a nucleic acid encoding a butyrylcholinesterase variant containing the nucleic acid sequence SEQ ID NO: 81, or a functional fragment thereof. The invention further provides a nucleic acid encoding a butyrylcholinesterase variant containing the nucleic acid sequence SEQ ID NO: 83, or a functional fragment thereof. The invention further provides a nucleic acid encoding a butyrylcholinesterase variant containing the nucleic acid sequence SEQ ID NO: 85, or a functional fragment thereof. The invention further provides a nucleic acid encoding a butyrylcholinesterase variant containing the nucleic acid sequence SEQ ID NO: 87, or a functional fragment thereof. The invention further provides a nucleic acid encoding a butyrylcholinesterase variant containing the nucleic acid sequence SEQ ID NO: 89, or a functional fragment thereof. The invention further provides a nucleic acid encoding a butyrylcholinesterase variant containing the nucleic acid sequence SEQ ID NO: 91, or a functional fragment thereof. The invention further provides a nucleic acid encoding a butyrylcholinesterase variant containing the nucleic acid sequence SEQ ID NO: 93, or a functional fragment thereof. The invention further provides a nucleic acid encoding a butyrylcholinesterase variant containing the nucleic acid sequence SEQ ID NO: 95, or a functional fragment thereof. The invention further provides a nucleic acid encoding a butyrylcholinesterase variant containing the nucleic acid sequence SEQ ID NO: 97, or a functional fragment thereof. The invention further provides a nucleic acid encoding a butyrylcholinesterase variant containing the nucleic acid sequence SEQ ID NO: 99, or a functional fragment thereof. The invention further provides a nucleic acid encoding a butyrylcholinesterase variant containing the nucleic acid sequence SEQ ID NO: 101, or a functional fragment thereof. The invention further provides a nucleic acid encoding a butyrylcholinesterase variant containing the nucleic acid sequence SEQ ID NO: 103, or a functional fragment thereof. The invention further provides a nucleic acid encoding a butyrylcholinesterase variant containing the nucleic acid sequence SEQ ID NO: 105, or a functional fragment thereof. The invention further provides a nucleic acid encoding a butyrylcholinesterase variant containing the nucleic acid sequence SEQ ID NO: 107, or a functional fragment thereof. The invention further provides a nucleic acid encoding a butyrylcholinesterase variant containing the nucleic acid sequence SEQ ID NO: 109, or a functional fragment thereof. The invention further provides a nucleic acid encoding a butyrylcholinesterase variant containing the nucleic acid sequence SEQ ID NO: 111, or a functional fragment thereof. The invention further provides a nucleic acid encoding a butyrylcholinesterase variant containing the nucleic acid sequence SEQ ID NO: 113, or a functional fragment thereof. The invention further provides a nucleic acid encoding a butyrylcholinesterase variant containing the nucleic acid sequence SEQ ID NO: 115, or a functional fragment thereof. The invention further provides a nucleic acid encoding a butyrylcholinesterase variant containing the nucleic acid sequence SEQ ID NO: 117, or a functional fragment thereof. The invention further provides a nucleic acid encoding a butyrylcholinesterase variant containing the nucleic acid sequence SEQ ID NO: 119, or a functional fragment thereof. The invention further provides a nucleic acid encoding a butyrylcholinesterase variant containing the nucleic acid sequence SEQ ID NO: 121, or a functional fragment thereof. The invention further provides a nucleic acid encoding a butyrylcholinesterase variant containing the nucleic acid sequence SEQ ID NO: 123, or a functional fragment thereof. The invention further provides a nucleic acid encoding a butyrylcholinesterase variant containing the nucleic acid sequence SEQ ID NO: 125, or a functional fragment thereof. The invention further provides a nucleic acid encoding a butyrylcholinesterase variant containing the nucleic acid sequence SEQ ID NO: 127, or a functional fragment thereof. The invention further provides a nucleic acid encoding a butyrylcholinesterase variant containing the nucleic acid sequence SEQ ID NO: 129, or a functional fragment thereof. The invention further provides a nucleic acid encoding a butyrylcholinesterase variant containing the nucleic acid sequence SEQ ID NO: 131, or a functional fragment thereof. The invention further provides a nucleic acid encoding a butyrylcholinesterase variant containing the nucleic acid sequence SEQ ID NO: 133, or a functional fragment thereof. The invention further provides a nucleic acid encoding a butyrylcholinesterase variant containing the nucleic acid sequence SEQ ID NO: 135, or a functional fragment thereof. The invention further provides a nucleic acid encoding a butyrylcholinesterase variant containing the nucleic acid sequence SEQ ID NO: 137, or a functional fragment thereof. The invention further provides a nucleic acid encoding a butyrylcholinesterase variant containing the nucleic acid sequence SEQ ID NO: 139, or a functional fragment thereof. The invention further provides a nucleic acid encoding a butyrylcholinesterase variant containing the nucleic acid sequence SEQ ID NO: 141, or a functional fragment thereof. The invention further provides a nucleic acid encoding a butyrylcholinesterase variant containing the nucleic acid sequence SEQ ID NO: 143, or a functional fragment thereof. The invention further provides a nucleic acid encoding a butyrylcholinesterase variant containing the nucleic acid sequence SEQ ID NO: 145, or a functional fragment thereof. The invention further provides a nucleic acid encoding a butyrylcholinesterase variant containing the nucleic acid sequence SEQ ID NO: 147, or a functional fragment thereof. The invention further provides a nucleic acid encoding a butyrylcholinesterase variant containing the nucleic acid sequence SEQ ID NO: 149, or a functional fragment thereof. The invention further provides a nucleic acid encoding a butyrylcholinesterase variant containing the nucleic acid sequence SEQ ID NO: 151, or a functional fragment thereof. The invention further provides a nucleic acid encoding a butyrylcholinesterase variant containing the nucleic acid sequence SEQ ID NO: 153, or a functional fragment thereof. The invention further provides a nucleic acid encoding a butyrylcholinesterase variant containing the nucleic acid sequence SEQ ID NO: 155, or a functional fragment thereof. The invention further provides a nucleic acid encoding a butyrylcholinesterase variant containing the nucleic acid sequence SEQ ID NO: 157, or a functional fragment thereof. The invention further provides a nucleic acid encoding a butyrylcholinesterase variant containing the nucleic acid sequence SEQ ID NO: 159, or a functional fragment thereof. The invention further provides a nucleic acid encoding a butyrylcholinesterase variant containing the nucleic acid sequence SEQ ID NO: 161, or a functional fragment thereof. The invention further provides a nucleic acid encoding a butyrylcholinesterase variant containing the nucleic acid sequence SEQ ID NO: 163, or a functional fragment thereof. The invention further provides a nucleic acid encoding a butyrylcholinesterase variant containing the nucleic acid sequence SEQ ID NO: 165, or a functional fragment thereof. The invention further provides a nucleic acid encoding a butyrylcholinesterase variant containing the nucleic acid sequence SEQ ID NO: 167, or a functional fragment thereof. The invention further provides a nucleic acid encoding a butyrylcholinesterase variant containing the nucleic acid sequence SEQ ID NO: 169, or a functional fragment thereof. The invention further provides a nucleic acid encoding a butyrylcholinesterase variant containing the nucleic acid sequence SEQ ID NO: 171, or a functional fragment thereof. The invention further provides a nucleic acid encoding a butyrylcholinesterase variant containing the nucleic acid sequence SEQ ID NO: 173, or a functional fragment thereof. The invention further provides a nucleic acid encoding a butyrylcholinesterase variant containing the nucleic acid sequence SEQ ID NO: 175, or a functional fragment thereof. The invention further provides a nucleic acid encoding a butyrylcholinesterase variant containing the nucleic acid sequence SEQ ID NO: 177, or a functional fragment thereof. The invention further provides a nucleic acid encoding a butyrylcholinesterase variant containing the nucleic acid sequence SEQ ID NO: 179, or a functional fragment thereof. The invention further provides a nucleic acid encoding a butyrylcholinesterase variant containing the nucleic acid sequence SEQ ID NO: 181, or a functional fragment thereof. The invention further provides a nucleic acid encoding a butyrylcholinesterase variant containing the nucleic acid sequence SEQ ID NO: 183, or a functional fragment thereof. The invention further provides a nucleic acid encoding a butyrylcholinesterase variant containing the nucleic acid sequence SEQ ID NO: 185, or a functional fragment thereof. The invention further provides a nucleic acid encoding a butyrylcholinesterase variant containing the nucleic acid sequence SEQ ID NO: 187, or a functional fragment thereof. The invention further provides a nucleic acid encoding a butyrylcholinesterase variant containing the nucleic acid sequence SEQ ID NO: 189, or a functional fragment thereof. The invention further provides a nucleic acid encoding a butyrylcholinesterase variant containing the nucleic acid sequence SEQ ID NO: 191, or a functional fragment thereof. The invention further provides a nucleic acid encoding a butyrylcholinesterase variant containing the nucleic acid sequence SEQ ID NO: 193, or a functional fragment thereof. The invention further provides a nucleic acid encoding a butyrylcholinesterase variant containing the nucleic acid sequence SEQ ID NO: 195, or a functional fragment thereof.

Cholinesterases are ubiquitous, polymorphic carboxylase Type B enzymes capable of hydrolyzing the neurotransmitter acetylcholine and numerous ester-containing compounds. Two major cholinesterases are acetylcholinesterase and butyrylcholinesterase. Butyrylcholinesterase catalyzes the hydrolysis of a number of choline esters as shown:

$$Acetylcholine + H2O {\to }^{BChE} Choline + Corresponding Acid$$

Butyrylcholinesterase preferentially uses butyrylcholine and benzoylcholine as substrates. Butyrylcholinesterase is found in mammalian blood plasma, liver, pancreas, intestinal mucosa and the white matter of the central nervous system. The human gene encoding butyrylcholinesterase is located on chromosome 3 and over thirty naturally occuring genetic variations of butyrylcholinesterase are known. The butyrylcholinesterase polypeptide is 574 amino acids in length and encoded by 1,722 base pairs of coding sequence. Three naturally occuring butyrylcholinesterase variations are the atypical alleles referred to as A variant, the J variant and the K variant. The A variant has an D70G mutation and is rare (0.5% allelic frequency), while the J variant has a E497V mutation and has only been found in one family. The K variant has a point mutation at nucleotide 1615, which results in an A539T mutation and has an allelic frequency of around 12% in Caucasians.

In addition to the naturally-occurring human variations of butyrylcholinesterase, a number of species variations are known. The amino acid sequence of cat butyrylcholinesterase is 88% identical with human butyrylcholinesterase. Of the seventy amino acids that differ, three are located in the active site gorge and are termed A277L, P285L and F398I. Similarly, horse butyrylcholinesterase has three amino acid differences in the active site compared with human butyrylcholinesterase, which are A277V, P285L and F398I. The amino acid sequence of rat butyrylcholinesterase contains 6 amino acid differences in the active site gorge, which are A277K, V280L, T284S, P285I, L286R and V288I.

Naturally occurring human butyrylcholinesterase variations, species variations as well as recombinantly prepared mutations have previously been described by Xie et al., Molecular Pharmacology 55: 83-91 (1999). A butyrylcholinesterase variant of the invention can be prepared by a variety of methods well known in the art. If desired, random mutagenesis can be performed to prepare a butyrylcholinesterase variant of the invention. Alternatively, as disclosed herein, random mutagenesis focused in discrete regions based on the information obtained from structural, biochemical and modeling methods described herein can be performed to target those amino acids predicted to be important for catalytic activity. For example, molecular modeling of a substrate in the active site of butyrylcholinesterase can be utilized to predict amino acid alterations that allow for higher catalytic efficiency based on a better fit between the enzyme and its substrate. In addition, molecular modeling can be used to predict amino acid alterations that decrease steric hinderance between the enzyme and substrate. Based on studies with cocaine as a substrate, residues predicted to be important for hydrolysis activity include 8 hydrophobic gorge residues and the catalytic triad residues. Furthermore, it is understood that amino acid alterations of residues important for the functional structure of a butyrylcholinesterase variant, which include the cysteine residues ⁶⁵Cys-⁹²Cys, ²⁵²Cys⁻²⁶³Cys, and ⁴⁰⁰Cys⁻⁵¹⁹Cys involved in intrachain disulfide bonds are generally not altered in the preparation of a butyrylcholinesterase variant that has hydrolysis activity.

Following mutagenesis of butyrylcholinesterase or a butyrylcholinesterase variant expression, purification and functional characterization of the butyrylcholinesterase variant can be performed by methods well known in the art.

A butyrylcholinesterase variant of the invention exhibits camptothecin conversion or hydrolysis activity. As disclosed herein, a butyrylcholinesterase variant of the invention can have enhanced camptothecin conversion or hydrolysis activity and can be used to treat cancer. A polypeptide having minor modifications compared to a butyrylcholinesterase variant of the invention is encompassed by the invention so long as equivalent camptothecin conversion or hydrolysis activity is retained. In addition, functional fragments of a butyrylcholinesterase variant that still retain some or all of the camptothecin conversion or hydrolysis activity of the parent butyrylcholinesterase variant are similarly included in the invention. Similarly, functional fragments of nucleic acids, which encode functional fragments of a butyrylcholinesterase variant of the invention are similarly encompassed by the invention.

A functional fragment of a butyrylcholinesterase or a butyrylcholinesterase variant of the invention can be prepared by recombinant methods involving expression of a nucleic acid molecule encoding the butyrylcholinesterase variant or functional fragment thereof, followed by isolation of the variant or functional fragment thereof by routine biochemical methods described herein. It is understood that functional fragments can also be prepared by enzymatic or chemical cleavage of the full length butyrylcholinesterase variant. Methods for enzymatic and chemical cleavage and for purification of the resultant peptide fragments are well known in the art (see, for example, Deutscher, Methods in Enzymology, Vol. 182, “Guide to Protein Purification,” San Diego: Academic Press, Inc. (1990), which is incorporated herein by reference).

Furthermore, functional fragments of a butyrylcholinesterase variant can be produced by chemical synthesis. If desired, such molecules can be modified to include D-stereoisomers, non-naturally occurring amino acids, and amino acid analogs and mimetics in order to optimize their functional activity, stability or bioavailability. Examples of modified amino acids and their uses are presented in Sawyer, Peptide Based Drug Design, ACS, Washington (1995) and Gross and Meienhofer, The Peptides: Analysis, Synthesis, Biology, Academic Press, Inc., New York (1983), both of which are incorporated herein by reference.

If desired, random segments of a butyrylcholinesterase variant can be prepared and tested in the assays described herein. A fragment having any desired boundaries and modifications compared to the amino acid sequence of the reference butyrylcholinesterase or butyrylcholinesterase variant of the invention can be prepared. Alternatively, available information obtained by the structural, biochemical and modeling methods described herein can be used to prepare only those fragments of a butyrylcholinesterase variant that are likely to retain the camptothecin conversion or hydrolysis activity of the parent variant. As described herein, residues predicted to be important for camptothecin conversion or hydrolysis activity include 8 hydrophobic gorge residues and the catalytic triad residues. Furthermore, residues important for the functional structure of a butyrylcholinesterase variant include the cysteine residues ⁶⁵Cys-⁹²Cys, ²⁵²Cys⁻²⁶³Cys, and ⁴⁰⁰Cys⁻⁵¹⁹Cys involved in intrachain disulfide bonds. A functional fragment can include non-peptidic structural elements that serve to mimic structurally or functionally important residues of the reference variant.

Also included as butyrylcholinesterase variants of the invention are fusion proteins that result from linking a butyrylcholinesterase variant or functional fragment thereof to a heterologous protein, such as a therapeutic protein, as well as fusion constructs of nucleic acids encoding such fusion proteins. Fragments of nucleic acids that can hybridize to a butyrylcholinesterase variant or functional fragment thereof are useful, for example, as hybridization probes and are also encompassed by the claimed invention.

The invention further provides butyrylcholinesterase variants, or functional fragment thereof, that contains an antibody or antibody fragment. A butyrylcholinesterase variant of the invention can be fused to any antibody or antibody fragment. For example, a butyrylcholinesterase variant of the invention can be fused to an antibody or antibody fragment that binds to a tumor-associated antigen. In this way the butyrylcholinesterase variant can be delivered directly to a tumor which can result in a decreased number of side effects. Several antigens are known to be over-expressed in tumor cells or expressed exclusively in tumor cells. These tumor associated antigens include, for example, Lewis Y (Siegall, C., Semin. Cancer Biol. 6: 289-295 (1995)), carcinoembryonic antigen (CEA)(Watine et al., Dis. Colon Rectum 44: 1791-1799 (2001)), tetraspanin L6 (Kaneko et al., Am. J. Gastroenterol. 96: 3457-3458 (2001)), 17-1A (Indar et al., J.R. Coll. Surg. Edinb. 47: 458-474 (2002)), mucin-1 (MUC-1) (Segal-Eiras and Croce, Allerg. Immunopath. 25: 176-181 (1997)), epidermal growth factor receptor (EGFR) (Bookman, M., Semin. Oncol. 25: 381-396 (1998)), cancer antigen 125 (CA 125) (Cherry and Vacchiano, Semin. Oncol. Nurs. 18: 167-173 (2002)), p97 (Srivastava, P., Curr. Opin. Immunol. 3: 654-658 (1991)), melanoma antigen gene (MAGE) (Barker and Salehi, J. Neurosci. Res. 67: 705-712 (2002)), CD20 (Kosmas et al., Leukemia 16: 2004-2015 (2002)), CD33 (Countouriotis et al., Stem Cells 20: 215-229 (2002)), ganglioside GD2 (Ragupathi, G., Cancer Immunol. Imunother. 43: 152-157 (1996)), and ganglioside GD3 (Ragupathi, G., supra (1996)).

A butyrylcholinesterase variant of the invention can be fused to an internalizing antibody or antibody fragment or a non-internalizing antibody or antibody fragment. When fused to a non-internalizing antibody or antibody fragment, the butyrylcholinesterase variant can be internalized through binding to a cell surface polypeptide that undergoes internalization. For example, a butyrylcholinesterase variant of the invention can be fused to an antibody directed to a receptor that undergoes internalization.

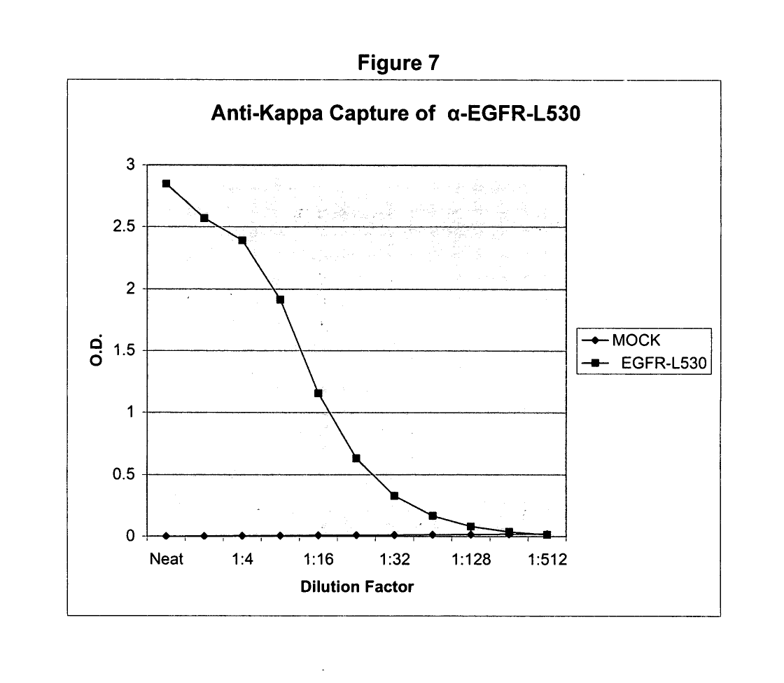

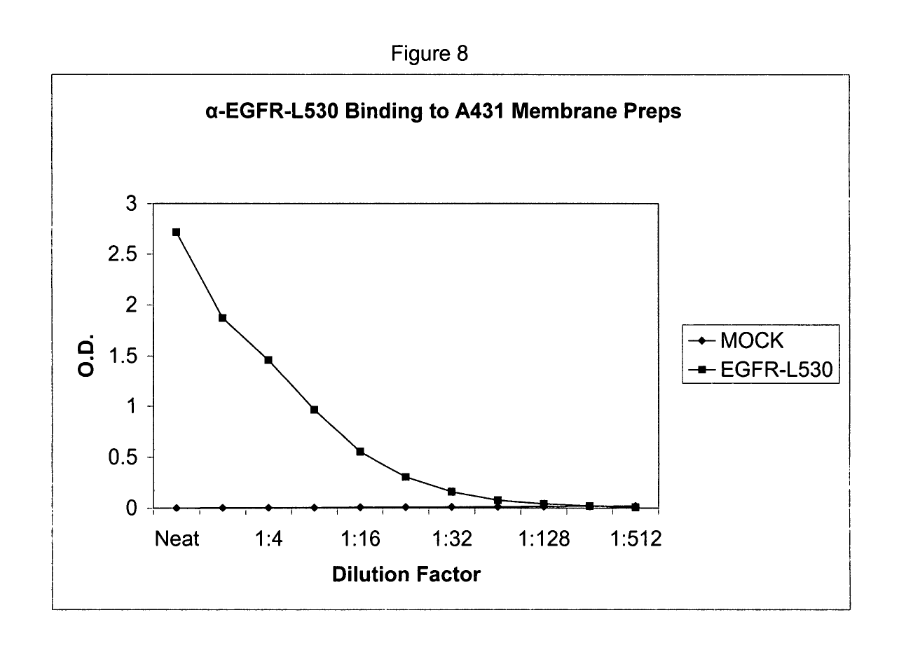

The invention provides a butyrylcholinesterase variant where the antibody or antibody fragment specifically binds a cell surface receptor. In one embodiment, the invention provides a butyrylcholinesterase variant where the antibody or antibody fragment specifically binds epidermal growth factor receptor (EGFR). The EGFR is known to be up-regulated in several tumor cell types, for example, in breast cancer cells. In various related embodiments, the invention provides a butyrylcholinesterase variant where the antibody or antibody fragment contains an amino acid sequence selected from a linker variant, hinge variant, and a synthetic linker variant. In one embodiment, the invention provides a butyrylcholinesterase variant where the antibody or antibody fragment contains an amino acid sequence as set forth in SEQ ID NOS: 18 and 20. ELISA results using a model antibody are shown in FIG. 7 and FIG. 8.

In a further embodiment, the invention provides a butyrylcholinesterase variant where the antibody or antibody fragment specifically binds CD20. CD20 is a non-glycosylated phosphoprotein on the B-cell surface. CD20-antibody complexes do not internalize, thereby allowing cell-surface bound immunoglobulin to interact with effector cells or complement for a longer time. In one embodiment, the invention provides a butyrylcholinesterase variant where the antibody or antibody fragment contains an amino acid sequence as set forth in SEQ ID NOS: 198 and 200, and specifically binds CD20. CD20 is known to be up-regulated in several tumor cell types, for example, B cell lymphomas such as Non-Hodgkin's lymphoma as well in various autoimmune conditions.

Fusions between a butyrylcholinesterase variant and an antibody or antibody fragment can be used for targeted tumor cell-specific butyrylcholoinesterase mediated toxicity using a process called antibody-directed enzyme prodrug therapy (ADEPT) (Jung, M., Mini Rev. Med. Chem. 1: 399-407 (2001); Bagshawe, K. D., Mol. Med. Today 1: 424-431 (1995); and Senter, P. D., FASEB J 4: 188-193 (1990)). A related method called viral-directed enzyme prodrug therapy (VDEPT) can also be utilized. An example of a fusion between a butyrylcholinesterase variant and an antibody or antibody fragment that can be used for targeted tumor cell-specific butyrylcholoinesterase mediated toxicity is set forth as amino acid sequence designated SEQ ID NO: 202 and corresponding nucleotide sequence set forth as SEQ ID NO: 201, which corresponds to an anti-CD20 VH-CH1 hinge cys L530 BChE.4-1 heavy chain construct. The butyrylcholinesterase variant designated SEQ ID NO: 202 is a fusion protein that contains a heavy chain comprised of the anti-CD20 antibody variable heavy chain region with a cysteine-containing hinge region and the L530 functional fragment (SEQ ID NO: 204) of the butyrylcholinesterase variant designated SEQ ID NO: 180, which incorporates the 4-1 variant amino acids (H77F/F227A/P285N/V331A). VDEPT uses a viral vector to deliver an enzyme such as a butyrylcholinesterase variant of the invention. With such approaches, selective expression of an enzyme can efficiently activate non-toxic or moderately toxic prodrugs in tumor cells into highly toxic metabolic products resulting in enhanced anti-tumor activity and an improved therapeutic index. In order for these approaches to be successful the enzyme needs to be of high activity, for example, the butyrylcholinesterase variants of the invention can be used.

The butyrylcholinesterase variants of the invention were derived from libraries as disclosed in Example I. A library that is sufficiently diverse to contain a butyrylcholinesterase variant with enhanced camptothecin conversion or hydrolysis activity can be prepared by a variety of methods well known in the art. Those skilled in the art will know what size and diversity is necessary or sufficient for the intended purpose. For example, a library of butyrylcholinesterase variants can be prepared that contains each of the 19 amino acids not found in the reference butyrylcholinesterase at each of the approximately 573 amino acid positions and screening the resultant variant library for butyrylcholinesterase variants with enhanced camptothecin hydrolysis activity.

Alternatively, a focused library can be prepared utilizing the structural, biochemical and modeling information relating to butyrylcholinesterase as described herein. It is understood that any information relevant to the determination or prediction of residues or regions important for camptothecin conversion or hydrolysis activity or structural function of butyrylcholinesterase can be useful in the design of a focused library of butyrylcholinesterase variants of the invention that have enhanced camptothecin hydrolysis activity. Thus, the butyrylcholinesterase variants that make up the library of butyrylcholinesterase variants of the invention can contain amino acid alterations at amino acid positions located in regions determined or predicted to be important for camptothecin conversion or hydrolysis activity. A focused library of butyrylcholinesterase variants can be desirable as it significantly decreases the number of variants that need to be screened in order to identify a butyrylcholinesterase variant with enhanced activity by targeting amino acid alterations to regions determined or predicted to be important for activity.

Regions important for camptothecin conversion or hydrolysis activity of butyrylcholinesterase can be determined or predicted through a variety of methods known in the art and used to focus the synthesis of a library of butyrylcholinesterase variants. Related enzymes such as, for example, acetylcholinesterase and carboxylesterase, that share a high degree of sequence similarity and have biochemically similar catalytic properties can provide information regarding the regions important for catalytic activity of butyrylcholinesterase. For example, structural modeling can reveal the active site of an enzyme, which is a three-dimensional structure such as a cleft, gorge or crevice formed by amino acid residues generally located apart from each other in primary structure. Therefore, amino acid residues that make up regions of butyrylcholinesterase important for camptothecin conversion or hydrolysis activity can include residues located along the active site gorge. For a description of structural modeling of butyrylcholinesterase, see for example, Harel et al., Proc. Nat. Acad. Sci. USA 89: 10827-10831 (1992) and Soreq et al., Trends Biochem. Sci. 17(9): 353-358 (1992), which are incorporated herein by reference.

In addition to structural modeling of butyrylcholinesterase, biochemical data can be used to determine or predict regions of butyrylcholinesterase important for camptothecin conversion or hydrolysis activity when preparing a focused library of butyrylcholiesterase variants. In this regard, the characterization of naturally occurring butyrylcholinesterase variants with altered camptothecin conversion or hydrolysis activity is useful for identifying regions important for the catalytic activity of butyrylcholinesterase. Similarly, site-directed mutagenesis studies can provide data regarding catalytically important amino acid residues as reviewed, for example, in Schwartz et al., Pharmac. Ther. 67: 283-322 (1992), which is incorporated by reference.

To generate a library of butyrylcholinesterase variants of the invention distinct types of information can be used alone or combined to determine or predict a region of an amino acid sequence of butyrylcholinesterase important for camptothecin conversion or hydrolysis activity. For example, information based on structural modeling and biochemical data is combined to determine a region of an amino acid sequence of butyrylcholinesterase important for camptothecin conversion or hydrolysis activity. Because information obtained by a variety of methods can be combined to predict the catalytically active regions, one skilled in the art will appreciate that the regions themselves represent approximations rather than strict confines. As a result, a library of butyrylcholinesterases can contain butyrylcholinesterase variants that have amino acid alterations outside of the regions determined or predicted to be important for camptothecin conversion or hydrolysis activity. Similarly, a butyrylcholinesterase variant of the invention can have amino acid alterations outside of the regions determined or predicted to be important for camptothecin conversion or hydrolysis activity. Furthermore, a butyrylcholinesterase variant of the invention can have any other modification that does not significantly change its camptothecin conversion or hydrolysis activity. It is further understood that the number of regions determined or predicted to be important for camptothecin conversion or hydrolysis activity can vary based on the predictive methods used.

Once a number of regions has been identified by any method appropriate for determination of regions important for camptothecin hydrolysis, or combination thereof, each region can be randomized across some or all amino acid positions to create a library of variants containing the wild-type amino acid plus one or more of the other nineteen naturally occurring amino acids at one or more positions within each of the regions. Seven regions of an amino acid sequence of butyrylcholinesterase selected for the focused library of butyrylcholinesterase variants provided by the invention are shown in Table 1.

TABLE 1
Butyrylcholinesterase Regions Predicted to be Important for
Catalytic Efficiency.

| Region | Location | Length |
|---|---|---|
| 1 | 68-82 | 15 |
| 2 | 110-121 | 12 |
| 3 | 194-201 | 8 |
| 4 | 224-234 | 11 |
| 5 | 277-289 | 13 |
| 6 | 327-332 | 6 |
| 7 | 429-442 | 14 |

Methods for preparing libraries containing diverse populations of various types of molecules such as peptides, peptoids and peptidomimetics are well known in the art (see, for example, Ecker and Crooke, Biotechnology 13: 351-360 (1995), and Blondelle et al., Trends Anal. Chem. 14: 83-92 (1995), and the references cited therein, each of which is incorporated herein by reference; see, also, Goodman and Ro, Peptidomimetics for Drug Design, in “Burger's Medicinal Chemistry and Drug Discovery” Vol. 1 (ed. M. E. Wolff; John Wiley & Sons 1995), pages 803-861, and Gordon et al., J. Med. Chem. 37: 1385-1401 (1994), each of which is incorporated herein by reference). Where a molecule is a peptide, protein or fragment thereof, the molecule can be produced in vitro directly or can be expressed from a nucleic acid, which can be produced in vitro. Methods of synthetic peptide chemistry are well known in the art.

A library of butyrylcholinesterase variants can be produced, for example, by constructing a nucleic acid expression library encoding butyrylcholinesterase variants. Methods for producing such libraries are well known in the art (see, for example, Sambrook et al., Molecular Cloning: A Laboratory Manual (Cold Spring Harbor Laboratory Press 1989), which is incorporated herein by reference). A library of nucleic acids encoding butyrylcholinesterase variants can be composed of DNA, RNA or analogs thereof. A library containing RNA molecules can be constructed, for example, by synthesizing the RNA molecules chemically.

The generation of a library of nucleic acids encoding butyrylcholinesterase variants can be by any means desired by the user. Those skilled in the art will know what methods can be used to generate libraries of nucleic acids encoding butyrylcholinesterase variants. For example, butyrylcholinesterase variants can be generated by mutagenesis of nucleic acids encoding butyrylcholinesterase using methods well known to those skilled in the art (Molecular Cloning: A Laboratory Manual, Sambrook et al., eds., Cold Spring Harbor Press, Plainview, N.Y. (1989)). A library of nucleic acids encoding butyrylcholinesterase variants of the invention can be randomized to be sufficiently diverse to contain nucleic acids encoding every possible naturally occurring amino acid at each amino acid position of butyrylcholinesterase. Alternatively, a library of nucleic acids can be prepared such that it contains nucleic acids encoding every possible naturally occuring amino acid at each amino acid only at positions located within a region of butyrylcholinesterase predicted or determined to be important for camptothecin conversion or hydrolysis activity.

One or more mutations can be introduced into a nucleic acid molecule encoding a butyrylcholinesterase variant to yield a modified nucleic acid molecule using, for example, site-directed mutagenesis (see Wu (Ed.), Meth. In Enzymol. Vol. 217, San Diego: Academic Press (1993); Higuchi, “Recombinant PCR” in Innis et al. (Ed.), PCR Protocols, San Diego: Academic Press, Inc. (1990), each of which is incorporated herein by reference). Such mutagenesis can be used to introduce a specific, desired amino acid alteration. Thus, distinct libraries containing amino acid alterations in one or more of the regions determined to be important for camptothecin conversion or hydrolysis activity as well as a single library containing mutations in several or all of the regions can be prepared.

The efficient synthesis and expression of libraries of butyrylcholinesterase variants using oligonucleotide-directed mutagenesis can be accomplished as previously described by Wu et al., Proc. Natl. Acad. Sci. USA, 95: 6037-6042 (1998); Wu et al., J. Mol. Biol., 294: 151-162 (1999); and Kunkel, Proc. Natl. Acad. Sci. USA, 82: 488-492 (1985), which are incorporated herein by reference. Oligonucleotide-directed mutagenesis is a well-known and efficient procedure for systematically introducing mutations, independent of their phenotype and is, therefore, ideally suited for directed evolution approaches to protein engineering. To perform oligonucleotide-directed mutagenesis a library of nucleic acids encoding the desired mutations is hybridized to single-stranded uracil-containing template of the wild-type sequence. The methodology is flexible, permitting precise mutations to be introduced without the use of restriction enzymes, and is relatively inexpensive if oligonucleotides are synthesized using codon-based mutagenesis.

Codon-based synthesis or mutagenesis represents one method well known in the art for avoiding genetic redundancy while rapidly and efficiently producing a large number of alterations in a known amino acid sequence or for generating a diverse population of random sequences. This method is the subject matter of U.S. Pat. Nos. 5,264,563 and 5,523,388 and is also described in Glaser et al. J. Immunology 149: 3903-3913 (1992). Briefly, coupling reactions for the randomization of, for example, all twenty codons which specify the amino acids of the genetic code are performed in separate reaction vessels and randomization for a particular codon position occurs by mixing the products of each of the reaction vessels. Following mixing, the randomized reaction products corresponding to codons encoding an equal mixture of all twenty amino acids are then divided into separate reaction vessels for the synthesis of each randomized codon at the next position. If desired, equal frequencies of all twenty amino acids can be achieved with twenty vessels that contain equal portions of the twenty codons. Thus, it is possible to utilize this method to generate random libraries of the entire sequence of butyrylcholinesterase or focused libraries of the regions determined or predicted to be important for camptothecin conversion or hydrolysis activity.

Variations to the above synthesis method also exist and include, for example, the synthesis of predetermined codons at desired positions and the biased synthesis of a predetermined sequence at one or more codon positions as described by Wu et al, supra, 1998. Biased synthesis involves the use of two reaction vessels where the predetermined or parent codon is synthesized in one vessel and the random codon sequence is synthesized in the second vessel. The second vessel can be divided into multiple reaction vessels such as that described above for the synthesis of codons specifying totally random amino acids at a particular position. Alternatively, a population of degenerate codons can be synthesized in the second reaction vessel such as through the coupling of NNG/T nucleotides or NNX/X where N is a mixture of all four nucleotides. Following synthesis of the predetermined and random codons, the reaction products in each of the two reaction vessels are mixed and then redivided into an additional two vessels for synthesis at the next codon position.

A modification to the above-described codon-based synthesis for producing a diverse number of variant sequences can similarly be employed for the production of the libraries of butyrylcholinesterase variants described herein. This modification is based on the two vessel method described above which biases synthesis toward the parent sequence and allows the user to separate the variants into populations containing a specified number of codon positions that have random codon changes.

Briefly, this synthesis is performed by continuing to divide the reaction vessels after the synthesis of each codon position into two new vessels. After the division, the reaction products from each consecutive pair of reaction vessels, starting with the second vessel, is mixed. This mixing brings together the reaction products having the same number of codon positions with random changes. Synthesis proceeds by then dividing the products of the first and last vessel and the newly mixed products from each consecutive pair of reaction vessels and redividing into two new vessels. In one of the new vessels, the parent codon is synthesized and in the second vessel, the random codon is synthesized. For example, synthesis at the first codon position entails synthesis of the parent codon in one reaction vessel and synthesis of a random codon in the second reaction vessel. For synthesis at the second codon position, each of the first two reaction vessels is divided into two vessels yielding two pairs of vessels. For each pair, a parent codon is synthesized in one of the vessels and a random codon is synthesized in the second vessel. When arranged linearly, the reaction products in the second and third vessels are mixed to bring together those products having random codon sequences at single codon positions. This mixing also reduces the product populations to three, which are the starting populations for the next round of synthesis. Similarly, for the third, fourth and each remaining position, each reaction product population for the preceding position are divided and a parent and random codon synthesized.

Following the above modification of codon-based synthesis, populations containing random codon changes at one, two, three and four positions as well as others can be conveniently separated out and used based on the need of the individual. Moreover, this synthesis scheme also allows enrichment of the populations for the randomized sequences over the parent sequence since the vessel containing only the parent sequence synthesis is similarly separated out from the random codon synthesis.

This method can be used to synthesize a library of nucleic acids encoding butyrylcholinesterase variants having amino acid alterations in one or more regions of butyrylcholinesterase predicted to be important for camptothecin conversion or hydrolysis activity.

Alternatively, a library of nucleic acids encoding butyrylcholinesterase variants can also be generated using gene shuffling. Gene shuffling or DNA shuffling is a method for directed evolution that generates diversity by recombination (see, for example, Stemmer, Proc. Natl. Acad. Sci. USA 91: 10747-10751 (1994); Stemmer, Nature 370: 389-391 (1994); Crameri et al., Nature 391: 288-291 (1998); Stemmer et al., U.S. Pat. No. 5,830,721, issued Nov. 3, 1998). Gene shuffling or DNA shuffling is a method using in vitro homologous recombination of pools of selected mutant genes. For example, a pool of point mutants of a particular gene can be used. The genes are randomly fragmented, for example, using DNase, and reassembled by PCR. If desired, DNA shuffling can be carried out using homologous genes from different organisms to generate diversity (Crameri et al., supra, 1998). The fragmentation and reassembly can be carried out in multiple rounds, if desired. The resulting reassembled genes constitute a library of butyrylcholinesterase variants that can be used in the invention compositions and methods.

The invention library of nucleic acids encoding butyrylcholinesterase variants can be expressed in a variety of eukaryotic cells. For example, the nucleic acids can be expressed in mammalian cells, insect cells, plant cells, and non-yeast fungal cells. Mammalian cell lines useful for expressing the invention library of nucleic acids encoding butyrylcholinesterase variants include, for example, Chinese Hamster Ovary (CHO), human 293T and Human NIH 3T3 cell lines. Expression of the invention library of nucleic acids encoding butyrylcholinesterase variants can be achieved by both stable or transient cell transfection (see Example III, Table 5).

The incorporation of variant nucleic acids or heterologous nucleic acid fragments at an identical site in the genome functions to create isogenic cell lines that differ only in the expression of a particular variant or heterologous nucleic acid. Incorporation at a single site minimizes positional effects from integration at multiple sites in a genome that affect transcription of the mRNA encoded by the nucleic acid and complications from the incorporation of multiple copies or expression of more than one nucleic acid species per cell. Techniques known in the art that can be used to target a variant or a heterologous nucleic acid to a specific location in the genome include, for example, homologous recombination, retroviral targeting and recombinase-mediated targeting.

One approach for targeting variant or heterologous nucleic acids to a single site in the genome uses Cre recombinase to target insertion of exogenous DNA into the eukaryotic genome at a site containing a site specific recombination sequence (Sauer and Henderson, Proc. Natl. Acad. Sci. USA, 85: 5166-5170 (1988); Fukushige and Sauer, Proc. Natl. Acad. Sci. U.S.A. 89: 7905-7909 (1992); Bethke and Sauer, Nuc. Acids Res., 25: 2828-2834 (1997)). In addition to Cre recombinase, Flp recombinase can also be used to target insertion of exogenous DNA into a particular site in the genome (Dymecki, Proc. Natl. Acad. Sci. U.S.A. 93: 6191-6196 (1996)). It is understood that any combination of site-specific recombinase and corresponding recombination site can be used in methods of the invention to target a nucleic acid to a particular site in the genome.

A suitable recombinase can be encoded on a vector that is co-transfected with a vector containing a nucleic acid encoding a butyrylcholinesterase variant. Alternatively, the expression element of a recombinase can be incorporated into the same vector expressing a nucleic acid encoding a butyrylcholinesterase variant. In addition to simultaneously transfecting the nucleic acid encoding a recombinase with the nucleic acids encoding a butyrylcholinesterase variant, a vector encoding the recombinase can be transfected into a cell, and the cells can be selected for expression of recombinase. A cell stably expressing the recombinase can subsequently be transfected with nucleic acids encoding variant nucleic acids.

The precise site-specific DNA recombination mediated by Cre recombinase can be used to create stable mammalian transformants containing a single copy of exogenous DNA encoding a butyrylcholinesterase variant. As exemplified below, the frequency of Cre-mediated targeting events can be enhanced substantially using a modified doublelox strategy. The doublelox strategy is based on the observation that certain nucleotide changes within the core region of the lox site alter the site selection specificity of Cre-mediated recombination with little effect on the efficiency of recombination (Hoess et al., Nucleic Acids Res. 14: 2287-2300 (1986)). Incorporation of loxP and an altered loxP site, termed lox511, in both the targeting vector and the host cell genome results in site-specific recombination by a double crossover event. The doublelox approach increases the recovery of site-specific integrants by 20-fold over the single crossover insertional recombination, increasing the absolute frequency of site-specific recombination such that it exceeds the frequency of illegitimate recombination (Bethke and Sauer, Nuc. Acids Res., 25: 2828-2834 (1997)).

Following the expression of a library of butyrylcholinesterase variants in a mammalian cell line, randomly selected clones can be sequenced and screened for increased catalytic activity. Methods for sequencing selected clones are well known to those of skill in the art and are described, for example, in Sambrook et al., supra, 1992, and in Ansubel et al., supra, 1998. Selecting a suitable method for measuring the camptothecin conversion or hydrolysis activity of a butyrylcholinesterase variant depends on a variety of factors such as, for example, the amount of the butyrylcholinesterase variant that is available. The camptothecin conversion or hydrolysis activity activity of a butyrylcholinesterase variant can be measured, for example, by spectrophotometry, by a microtiter-based assay utilizing a polyclonal anti-butyrylcholinesterase antibody to uniformly capture the butyrylcholinesterase variants and by high-performance liquid chromatography (HPLC).

Enhanced camptothecin conversion or hydrolysis activity of a butyrylcholinesterase variant compared to butyrylcholinesterase can be determined by a comparison of catalytic efficiencies as determined using assays known in the art and described herein. For example, the camptothecin conversion or hydrolysis activity of a butyrylcholinesterase variant can be determined using an o-nitrophenyl acetate hydrolysis assay (see Example II), a CPT-11 conversion to SN-38 HPLC assay (see Example III), or a cytotoxicity assay (see Example IV). To ensure that a library of butyrylcholinesterase variants has been screened exhaustively, screening of each library can be continued until clones encoding identical butyrylcholinesterase amino acid alterations have been identified on multiple occasions.

Clones expressing a butyrylcholinesterase variant with increased camptothecin hydrolysis activity can be used to established larger-scale cultures suitable for purifying larger quantities of the butyrylcholinesterase. A butyrylcholinesterase variant of interest can be cloned into an expression vector and used to transfect a cell line, which can subsequently be expanded. Those skilled in the art will know what type of expression vector is suitable for a particular application. A butyrylcholinesterase variant exhibiting increased camptothecin conversion or hydrolysis activity can be cloned, for example, into an expression vector carrying a gene that confers resistance to a particular chemical agent to allow positive selection of the transfected cells. An expression vector suitable for transfection of, for example, mammalian cell lines can contain a promoter such as the cytomegalovirus (CMV) promoter for selection in mammalian cells. As desribed herein, a butyrylcholinesterase variant can be cloned into a mammalian expression vector and transiently transfected into human 293T cells. Expression vectors suitable for expressing a butyrylcholinesterase variant are well known in the art and commercially available.

Clones expressing butyrylcholinesterase variants can be selected and tested for camptothecin conversion or hydrolysis activity. Cells carrying clones exhibiting enhanced camptothecin conversion or hydrolysis activity can be expanded by routine cell culture systems to produce larger quantities of a butyrylcholinesterase variant of interest. The concentrated recombinant butyrylcholinesterase variant can be harvested and purified by methods well known in the art and described, for example, by Masson et al., Biochemistry 36: 2266-2277 (1997), which is incorporated herein by reference.

A butyrylcholinesterase variant having an increased serum half-life can be useful for testing a butyrylcholinesterase variant in a subject or treating cancer in an individual. Useful methods for increasing the serum half-life of a butyrylcholinesterase variant include, for example, conversion of the butyrylcholinesterase variant into a tetramer, covalently attaching synthetic and natural polymers such as polyethylene glycol (PEG) and dextrans to the truncated butyrylcholinesterase variant, liposome formulations, or expression of the enzyme as an Ig-fusion protein. As disclosed herein, conversion of a butyrylcholineserase variant into a tetramer can be achieved by co-transfecting the host cell line with the COLQ gene as well as by addition of poly-L-proline to the media of transfected cells. These and other methods known in the art for increasing the serum half-life of a butyrylcholinesterase variant are useful for testing a butyrylcholinesterase variant in an animal subject or treating cancer in an individual.

The invention provides a method of converting a camptothecin derivative to a topoisomerase inhibitor by contacting the camptothecin derivative with a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, under conditions that allow conversion of a camptothecin derivative to a topoisomerase inhibitor. In one embodiment the topoisomerase inhibitor is SN-38. In a further embodiment the camptothecin derivative is CPT-11. In one embodiment, the butyrylcholinesterase variant exhibits a two-fold or greater increase in conversion capability compared to butyrylcholinesterase, a ten-fold or greater increase in conversion capability compared to butyrylcholinesterase or a fifty-fold or more enhanced conversion capability compared to butyrylcholinesterase, a one hundred-fold or more enhanced conversion capability compared to butyrylcholinesterase, a two hundred-fold or more enhanced conversion capability compared to butyrylcholinesterase, a three hundred-fold or more enhanced conversion capability compared to butyrylcholinesterase. a four hundred-fold or more enhanced conversion capability compared to butyrylcholinesterase, a five hundred-fold or more enhanced conversion capability compared to butyrylcholinesterase, a one thousand-fold or more enhanced conversion capability compared to butyrylcholinesterase, a fifteen hundred-fold or more enhanced conversion capability compared to butyrylcholinesterase, a two thousand-fold or more enhanced conversion capability compared to butyrylcholinesterase, a three thousand-fold or more enhanced conversion capability compared to butyrylcholinesterase.

In one embodiment, the invention provides a method of converting a camptothecin derivative to a topoisomerase inhibitor by contacting the camptothecin derivative with a butyrylcholinesterase variant having the sequence as shown in SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, under conditions that allow conversion of a camptothecin derivative to a topoisomerase inhibitor. For example, in one embodiment the invention provides a method of converting a camptothecin derivative to a topoisomerase inhibitor by contacting the camptothecin derivative with a butyrylcholinesterase variant having the amino acid sequence as shown in SEQ ID NO: 2, or functional fragment thereof, under conditions that allow conversion of a camptothecin derivative to a topoisomerase inhibitor. In another embodiment, the butyrylcholinesterase variant has the amino acid sequence as shown in SEQ ID NO: 4, or functional fragment thereof. In a further embodiment, the butyrylcholinesterase variant has the amino acid sequence as shown in SEQ ID NO: 6, or functional fragment thereof. In a still further embodiment, the butyrylcholinesterase variant has the amino acid sequence as shown in SEQ ID NO: 8, or functional fragment thereof. In another embodiment, the butyrylcholinesterase variant has the amino acid sequence as shown in SEQ ID NO: 10, or functional fragment thereof. In a further embodiment, the butyrylcholinesterase variant has the amino acid sequence as shown in SEQ ID NO: 12, or functional fragment thereof. In another embodiment, the butyrylcholinesterase variant has the amino acid sequence as shown in SEQ ID NO: 14, or functional fragment thereof. In another embodiment, the butyrylcholinesterase variant has the amino acid sequence as shown in SEQ ID NO: 24, or functional fragment thereof. In another embodiment, the butyrylcholinesterase variant has the amino acid sequence as shown in SEQ ID NO: 26, or functional fragment thereof. In another embodiment, the butyrylcholinesterase variant has the amino acid sequence as shown in SEQ ID NO: 28, or functional fragment thereof. In another embodiment, the butyrylcholinesterase variant has the amino acid sequence as shown in SEQ ID NO: 30, or functional fragment thereof. In another embodiment, the butyrylcholinesterase variant has the amino acid sequence as shown in SEQ ID NO: 32, or functional fragment thereof. In another embodiment, the butyrylcholinesterase variant has the amino acid sequence as shown in SEQ ID NO: 34, or functional fragment thereof. In another embodiment, the butyrylcholinesterase variant has the amino acid sequence as shown in SEQ ID NO: 36, or functional fragment thereof. In another embodiment, the butyrylcholinesterase variant has the amino acid sequence as shown in SEQ ID NO: 38, or functional fragment thereof. In another embodiment, the butyrylcholinesterase variant has the amino acid sequence as shown in SEQ ID NO: 40, or functional fragment thereof. In another embodiment, the butyrylcholinesterase variant has the amino acid sequence as shown in SEQ ID NO: 42, or functional fragment thereof. In another embodiment, the butyrylcholinesterase variant has the amino acid sequence as shown in SEQ ID NO: 44, or functional fragment thereof. In another embodiment, the butyrylcholinesterase variant has the amino acid sequence as shown in SEQ ID NO: 46, or functional fragment thereof. In another embodiment, the butyrylcholinesterase variant has the amino acid sequence as shown in SEQ ID NO: 48, or functional fragment thereof. In another embodiment, the butyrylcholinesterase variant has the amino acid sequence as shown in SEQ ID NO: 50, or functional fragment thereof. In another embodiment, the butyrylcholinesterase variant has the amino acid sequence as shown in SEQ ID NO: 52, or functional fragment thereof. In another embodiment, the butyrylcholinesterase variant has the amino acid sequence as shown in SEQ ID NO: 54, or functional fragment thereof. In another embodiment, the butyrylcholinesterase variant has the amino acid sequence as shown in SEQ ID NO: 56, or functional fragment thereof. In another embodiment, the butyrylcholinesterase variant has the amino acid sequence as shown in SEQ ID NO: 58, or functional fragment thereof. In another embodiment, the butyrylcholinesterase variant has the amino acid sequence as shown in SEQ ID NO: 60, or functional fragment thereof. In another embodiment, the butyrylcholinesterase variant has the amino acid sequence as shown in SEQ ID NO: 62, or functional fragment thereof. In another embodiment, the butyrylcholinesterase variant has the amino acid sequence as shown in SEQ ID NO: 64, or functional fragment thereof. In another embodiment, the butyrylcholinesterase variant has the amino acid sequence as shown in SEQ ID NO: 66, or functional fragment thereof. In another embodiment, the butyrylcholinesterase variant has the amino acid sequence as shown in SEQ ID NO: 68, or functional fragment thereof. In another embodiment, the butyrylcholinesterase variant has the amino acid sequence as shown in SEQ ID NO: 70, or functional fragment thereof. In another embodiment, the butyrylcholinesterase variant has the amino acid sequence as shown in SEQ ID NO: 72, or functional fragment thereof. In another embodiment, the butyrylcholinesterase variant has the amino acid sequence as shown in SEQ ID NO: 74, or functional fragment thereof. In another embodiment, the butyrylcholinesterase variant has the amino acid sequence as shown in SEQ ID NO: 76, or functional fragment thereof. In another embodiment, the butyrylcholinesterase variant has the amino acid sequence as shown in SEQ ID NO: 78, or functional fragment thereof. In another embodiment, the butyrylcholinesterase variant has the amino acid sequence as shown in SEQ ID NO: 80, or functional fragment thereof. In another embodiment, the butyrylcholinesterase variant has the amino acid sequence as shown in SEQ ID NO: 82, or functional fragment thereof. In another embodiment, the butyrylcholinesterase variant has the amino acid sequence as shown in SEQ ID NO: 84, or functional fragment thereof. In another embodiment, the butyrylcholinesterase variant has the amino acid sequence as shown in SEQ ID NO: 86, or functional fragment thereof. In another embodiment, the butyrylcholinesterase variant has the amino acid sequence as shown in SEQ ID NO: 88, or functional fragment thereof. In another embodiment, the butyrylcholinesterase variant has the amino acid sequence as shown in SEQ ID NO: 90, or functional fragment thereof. In another embodiment, the butyrylcholinesterase variant has the amino acid sequence as shown in SEQ ID NO: 92, or functional fragment thereof. In another embodiment, the butyrylcholinesterase variant has the amino acid sequence as shown in SEQ ID NO: 94, or functional fragment thereof. In another embodiment, the butyrylcholinesterase variant has the amino acid sequence as shown in SEQ ID NO: 96, or functional fragment thereof. In another embodiment, the butyrylcholinesterase variant has the amino acid sequence as shown in SEQ ID NO: 98, or functional fragment thereof. In another embodiment, the butyrylcholinesterase variant has the amino acid sequence as shown in SEQ ID NO: 100, or functional fragment thereof. In another embodiment, the butyrylcholinesterase variant has the amino acid sequence as shown in SEQ ID NO: 102, or functional fragment thereof. In another embodiment, the butyrylcholinesterase variant has the amino acid sequence as shown in SEQ ID NO: 104, or functional fragment thereof. In another embodiment, the butyrylcholinesterase variant has the amino acid sequence as shown in SEQ ID NO: 106, or functional fragment thereof. In another embodiment, the butyrylcholinesterase variant has the amino acid sequence as shown in SEQ ID NO: 108, or functional fragment thereof. In another embodiment, the butyrylcholinesterase variant has the amino acid sequence as shown in SEQ ID NO: 110, or functional fragment thereof. In another embodiment, the butyrylcholinesterase variant has the amino acid sequence as shown in SEQ ID NO: 112, or functional fragment thereof. In another embodiment, the butyrylcholinesterase variant has the amino acid sequence as shown in SEQ ID NO: 114, or functional fragment thereof. In another embodiment, the butyrylcholinesterase variant has the amino acid sequence as shown in SEQ ID NO: 116, or functional fragment thereof. In another embodiment, the butyrylcholinesterase variant has the amino acid sequence as shown in SEQ ID NO: 118, or functional fragment thereof. In another embodiment, the butyrylcholinesterase variant has the amino acid sequence as shown in SEQ ID NO: 120, or functional fragment thereof. In another embodiment, the butyrylcholinesterase variant has the amino acid sequence as shown in SEQ ID NO: 122, or functional fragment thereof. In another embodiment, the butyrylcholinesterase variant has the amino acid sequence as shown in SEQ ID NO: 124, or functional fragment thereof. In another embodiment, the butyrylcholinesterase variant has the amino acid sequence as shown in SEQ ID NO: 126, or functional fragment thereof. In another embodiment, the butyrylcholinesterase variant has the amino acid sequence as shown in SEQ ID NO: 128, or functional fragment thereof. In another embodiment, the butyrylcholinesterase variant has the amino acid sequence as shown in SEQ ID NO: 130, or functional fragment thereof. In another embodiment, the butyrylcholinesterase variant has the amino acid sequence as shown in SEQ ID NO: 132, or functional fragment thereof. In another embodiment, the butyrylcholinesterase variant has the amino acid sequence as shown in SEQ ID NO: 134, or functional fragment thereof. In another embodiment, the butyrylcholinesterase variant has the amino acid sequence as shown in SEQ ID NO: 136, or functional fragment thereof. In another embodiment, the butyrylcholinesterase variant has the amino acid sequence as shown in SEQ ID NO: 138, or functional fragment thereof. In another embodiment, the butyrylcholinesterase variant has the amino acid sequence as shown in SEQ ID NO: 140, or functional fragment thereof. In another embodiment, the butyrylcholinesterase variant has the amino acid sequence as shown in SEQ ID NO: 142, or functional fragment thereof. In another embodiment, the butyrylcholinesterase variant has the amino acid sequence as shown in SEQ ID NO: 144, or functional fragment thereof. In another embodiment, the butyrylcholinesterase variant has the amino acid sequence as shown in SEQ ID NO: 146, or functional fragment thereof. In another embodiment, the butyrylcholinesterase variant has the amino acid sequence as shown in SEQ ID NO: 148, or functional fragment thereof. In another embodiment, the butyrylcholinesterase variant has the amino acid sequence as shown in SEQ ID NO: 150, or functional fragment thereof. In another embodiment, the butyrylcholinesterase variant has the amino acid sequence as shown in SEQ ID NO: 152, or functional fragment thereof. In another embodiment, the butyrylcholinesterase variant has the amino acid sequence as shown in SEQ ID NO: 154, or functional fragment thereof. In another embodiment, the butyrylcholinesterase variant has the amino acid sequence as shown in SEQ ID NO: 156, or functional fragment thereof. In another embodiment, the butyrylcholinesterase variant has the amino acid sequence as shown in SEQ ID NO: 158, or functional fragment thereof. In another embodiment, the butyrylcholinesterase variant has the amino acid sequence as shown in SEQ ID NO: 160, or functional fragment thereof. In another embodiment, the butyrylcholinesterase variant has the amino acid sequence as shown in SEQ ID NO: 162, or functional fragment thereof. In another embodiment, the butyrylcholinesterase variant has the amino acid sequence as shown in SEQ ID NO: 164, or functional fragment thereof. In another embodiment, the butyrylcholinesterase variant has the amino acid sequence as shown in SEQ ID NO: 166, or functional fragment thereof. In another embodiment, the butyrylcholinesterase variant has the amino acid sequence as shown in SEQ ID NO: 168, or functional fragment thereof. In another embodiment, the butyrylcholinesterase variant has the amino acid sequence as shown in SEQ ID NO: 170, or functional fragment thereof. In another embodiment, the butyrylcholinesterase variant has the amino acid sequence as shown in SEQ ID NO: 172, or functional fragment thereof. In another embodiment, the butyrylcholinesterase variant has the amino acid sequence as shown in SEQ ID NO: 174, or functional fragment thereof. In another embodiment, the butyrylcholinesterase variant has the amino acid sequence as shown in SEQ ID NO: 178, or functional fragment thereof. In another embodiment, the butyrylcholinesterase variant has the amino acid sequence as shown in SEQ ID NO: 180, or functional fragment thereof. In another embodiment, the butyrylcholinesterase variant has the amino acid sequence as shown in SEQ ID NO: 182, or functional fragment thereof. In another embodiment, the butyrylcholinesterase variant has the amino acid sequence as shown in SEQ ID NO: 184, or functional fragment thereof. In another embodiment, the butyrylcholinesterase variant has the amino acid sequence as shown in SEQ ID NO: 186, or functional fragment thereof. In another embodiment, the butyrylcholinesterase variant has the amino acid sequence as shown in SEQ ID NO: 188, or functional fragment thereof. In another embodiment, the butyrylcholinesterase variant has the amino acid sequence as shown in SEQ ID NO: 190, or functional fragment thereof. In another embodiment, the butyrylcholinesterase variant has the amino acid sequence as shown in SEQ ID NO: 192, or functional fragment thereof. In another embodiment, the butyrylcholinesterase variant has the amino acid sequence as shown in SEQ ID NO: 194, or functional fragment thereof. In another embodiment, the butyrylcholinesterase variant has the amino acid sequence as shown in SEQ ID NO: 196, or functional fragment thereof.

The invention also provides a method of converting a paxlitaxel prodrug to a paxlitaxel by contacting the paxlitaxel prodrug with a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 32, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, under conditions that allow conversion of a paxlitaxel prodrug to paxlitaxel. In one embodiment, the butyrylcholinesterase variant exhibits a two-fold or greater increase in conversion capability compared to butyrylcholinesterase, a ten-fold or greater increase in conversion capability compared to butyrylcholinesterase, a fifty-fold or more enhanced conversion capability compared to butyrylcholinesterase, a one hundred-fold or more enhanced conversion capability compared to butyrylcholinesterase, a two hundred-fold or more enhanced conversion capability compared to butyrylcholinesterase, a three hundred-fold or more enhanced conversion capability compared to butyrylcholinesterase, a four hundred-fold or more enhanced conversion capability compared to butyrylcholinesterase, a five hundred-fold or more enhanced conversion capability compared to butyrylcholinesteras, a one thousand-fold or more enhanced conversion capability compared to butyrylcholinesterase. a fifteen hundred-fold or more enhanced conversion capability compared to butyrylcholinesterase, a two thousand-fold or more enhanced conversion capability compared to butyrylcholinesterase, a three thousand-fold or more enhanced conversion capability compared to butyrylcholinesterase.

Paclitaxel prodrugs such as paclitaxel-2-ethylcarbonate (PC) have significant levels of antitumor activities in rodent models of human cancers. Paclitaxel (also known as TAXOL) was originally isolated from the bark of the Pacific yew tree and has been used in the treatment of several cancers including, for example, breast cancer, ovarian cancer, non-small cell lung cancer and Kaposi's sarcoma. The mechanism of action of this class of chemotherapeutic agents is the stabilization of tubulin. Serum carboxylesterases such as rat carboxylesterase has been shown to convert paclitaxel prodrugs, such as PC, to paxlitaxel. These serum carboxylesterases enhance the cytotoxic activity of PC on lung carcinoma and melanoma cell lines (Senter et al., Cancer Res. 56: 1471-1474 (1996)). Butyrylcholinesterase and butyrylcholinesterase variants of the invention can be used to convert paclitaxel prodrugs such as PC into active drugs useful for the treatment of cancer.

As described herein, a butyrylcholinesterase variant exhibiting increased camptothecin conversion or hydrolysis activity can convert or hydrolyze a substrate, such as a camptothecin derivative or paclitaxel, in vitro as well as in vivo. For example, a camptothecin derivative butyrylcholinesterase substrate can be contacted with a butyrylcholinesterase variant of the invention in vitro by adding the substrate to supernatant isolated from cultures of butyrylcholinesterase variant library clones. Alternatively, the butyrylcholinesterase variant can be purified prior to being contacted by the substrate. Appropriate medium conditions in which to contact a substrate such as a camptothecin derivative substrate with a butyrylcholinesterase variant of the invention are readily determined by those skilled in the art. As described below, butyrylcholinesterase variants from culture supernatants can further be immobilized using a capture agent, such as an antibody prior to being contacted with a substrate, which allows for removal of culture supernatant components and enables contacting of the immobilized variants with substrate in the absence of contaminants. Following contacting of a butyrylcholinesterase variant of the invention with a substrate, an activity of the variant enzyme can be measured. For example, after contacting a butyrylcholinesterase variant of the invention with a camptothecin derivative substrate, camptothecin conversion or hydrolysis activity can be measured by a variety of methods known in the art and described herein, such as high-performance liquid chromatography or a cytotoxicity assay.

The invention further provides a method of treating cancer by administering to an individual an effective amount of a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, exhibiting increased capability to convert a camptothecin derivative to a topoisomerase inhibitor compared to butyrylcholinesterase. In one embodiment, the cancer is metastatic colorectal cancer. In another embodiment the cancer is ovarian cancer. In a further embodiment the cancer is lung cancer, for example, small cell lung cancer or non-small cell lung cancer. In a still further embodiment the cancer is non-Hodgkin's lymphoma. In another embodiment, the cancer is a central nervous system cancer.

The invention also provides a method of treating cancer by administering to an individual an effective amount of a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 202 or 204 or functional fragment thereof, exhibiting increased capability to convert CPT-11 to a topoisomerase inhibitor compared to butyrylcholinesterase. In one embodiment, the topoisomerase inhibitor is SN-38.

The invention further provides a method of treating cancer by administering to an individual an effective amount of a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 202 or 204, or functional fragment thereof, exhibiting increased capability to convert a paclitaxel prodrug to paclitaxel compared to butyrylcholinesterase. In one embodiment, the cancer is metastatic colorectal cancer. In another embodiment the cancer is ovarian cancer. In another embodiment the cancer is breast cancer. In a further embodiment the cancer is lung cancer. In a still further embodiment the cancer is Kaposi's sarcoma.

Paclitaxel and camptothecin derivatives are known to be effective chemotherapeutic agents against a variety of cancers. For example, CPT-11 has been approved by the FDA for the treatment of colon cancer. Improvements in the hydrolysis of CPT-11 to SN-38 will aid in the usefulness of this drug and reduce side-effects in patients. For example, side-effects of CPT-11 treatment can include diarrhea, hair loss, nausea, vomiting, myelosuppression, hyperglycemia, alopecia and cholinergic symptoms (Moertel et al., Cancer Chemo. R. 56: 95-101 (1972); Muggia et al., Ca. Chemother. Rep. 56: 515-521 (1972)). In addition to colon cancer, these drugs have been tested in a variety of other cancers (see Hare et al., Cancer Chemtoher. Pharmacol. 39: 187-191 (1997), incorporated herein by reference).

The invention provides a method of treating cancer in an individual by administering a therapeutically effective amount of the butyrylcholinesterase variant. It is contemplated that a method of treating cancer in an individual by administering a therapeutically effective amount of the butyrylcholinesterase variant can be administration of a variant that further contains an antibody or antibody fragment, for example, the CD20 (SEQ ID NOS: 198 and 200) and EGF (SEQ ID NOS: 18 and 20) antibodies and corresponding fragments described herein. The dosage of a butyrylcholinesterase variant required to be effective depends, for example, on the route and form of administration, the potency and bio-active half-life of the molecule being administered, the weight and condition of the individual, and previous or concurrent therapies. The appropriate amount considered to be an effective dose for a particular application of the method can be determined by those skilled in the art, using the teachings and guidance provided herein. For example, the amount can be extrapolated from in vitro or in vivo butyrylcholinesterase assays described herein. One skilled in the art will recognize that the condition of the individual needs to be monitored throughout the course of treatment and that the amount of the composition that is administered can be adjusted accordingly.

For treating cancer, a therapeutically effective amount of a butyrylcholinesterase variant of the invention can be, for example, between about 0.1 mg/kg to 0.15 mg/kg body weight, for example, between about 0.15 mg/kg to 0.3 mg/kg, between about 0.3 mg/kg to 0.5 mg/kg or between about 1 mg/kg to 5 mg/kg, depending on the treatment regimen. Similarly, formulations that allow for timed-release of a butyrylcholinesterase variant would provide for the continuous release of a smaller amount of a butyrylcholinesterase variant to an individual treated for cancer. It is understood, that the dosage of a butyrylcholinesterase variant has to be adjusted based on the catalytic activity of the variant, such that a lower dose of a variant exhibiting significantly enhanced camptothecin conversion or hydrolysis activity can be administered compared to the dosage necessary for a variant with lower camptothecin conversion or hydrolysis activity.

A butyrylcholinesterase variant can be delivered systemically, such as intravenously or intraarterially. A butyrylcholinesterase variant can be provided in the form of isolated and substantially purified polypetides and polypeptide fragments in pharmaceutically acceptable formulations using formulation methods known to those of ordinary skill in the art. These formulations can be administered by standard routes, including for example, topical, transdermal, intraperitoneal, intracranial, intracerebroventricular, intracerebral, intravaginal, intrauterine, oral, rectal or parenteral (e.g., intravenous, intraspinal, subcutaneous or intramuscular) routes. In addition, a butyrylcholinesterase variant can be incorporated into biodegradable polymers allowing for sustained release of the compound useful for treating individual symptomatic of cancer. Biodegradable polymers and their use are described, for example, in detail in Brem et al., J. Neurosurg. 74: 441-446 (1991), which is incorporated herein by reference.

A butyrylcholinesterase variant can be administered as a solution or suspension together with a pharmaceutically acceptable medium. Such a pharmaceutically acceptable medium can be, for example, water, sodium phosphate buffer, phosphate buffered saline, normal saline or Ringer's solution or other physiologically buffered saline, or other solvent or vehicle such as a glycol, glycerol, an oil such as olive oil or an injectable organic ester. A pharmaceutically acceptable medium can additionally contain physiologically acceptable compounds that act, for example, to stabilize or increase the absorption of the butyrylcholinesterase variant. Such physiologically acceptable compounds include, for example, carbohydrates such as glucose, sucrose or dextrans; antioxidants such as ascorbic acid or glutathione; chelating agents such as EDTA, which disrupts microbial membranes; divalent metal ions such as calcium or magnesium; low molecular weight proteins; lipids or liposomes; or other stabilizers or excipients.

Formulations suitable for parenteral administration include aqueous and non-aqueous sterile injection solutions such as the pharmaceutically acceptable mediums described above. The solutions can additionally contain, for example, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

The butyrylcholinesterase variant of the invention can further be utilized in combination therapies with other therapeutic agents. Combination therapies that include a butyrylcholinesterase variant can consist of formulations containing the variant and the additional therapeutic agent individually in a suitable formulation. Alternatively, combination therapies can consist of fusion proteins, where the butyrylcholinesterase variant is linked to a heterologous protein, such as a therapeutic protein or antibody or antibody fragment.

In vivo modes of administration of antibody therapeutics can include intraperitoneal, intravenous and subcutaneous administration of a fusion polypeptide antibody or a functional fragment thereof. Dosages for antibody therapeutics are known or can be routinely determined by those skilled in the art. For example, such dosages are typically administered so as to achieve a plasma concentration from about 0.01 μg/ml to about 100 μg/ml, about 1-5 μg/ml or about 5 μg/ml. In terms of amount per body weight, these dosages typically correspond to about 0.1-300 mg/kg, about 0.2-200 mg/kg or about 0.5-20 mg/kg. Depending on the need, dosages can be administered once or multiple times over the course of the treatment. Generally, the dosage will vary with the age, condition, sex and extent of the pathology of the subject and should not be so high as to cause adverse side effects. Moreover, dosages can also be modulated by the physician during the course of the treatment to either enhance the treatment or reduce the potential development of side effects. Such procedures are known and routinely performed by those skilled in the art.

A butyrylcholinesterase variant of the invention also can be delivered to an individual by administering an encoding nucleic acid for the peptide or variant. The encoding nucleic acids for the butyrylcholinesterase variant of the invention are useful in conjunction with a wide variety of gene therapy methods known in the art for delivering a therapeutically effective amount of the polypeptide or variant. Using the teachings and guidance provided herein, encoding nucleic acids for a butyrylcholinesterase variant can be incorporated into a vector or delivery system known in the art and used for delivery and expression of the encoding sequence to achieve a therapeutically effective amount. Applicable vector and delivery systems known in the art include, for example, retroviral vectors, adenovirus vectors, adenoassociated virus, ligand conjugated particles and nucleic acids for targeting, isolated DNA and RNA, liposomes, polylysine, and cell therapy, including hepatic cell therapy, employing the transplantation of cells modified to express a butyrylcholinesterase variant, as well as various other gene delivery methods and modifications known to those skilled in the art, such as those described in Shea et al., Nature Biotechnology 17: 551-554 (1999), which is incorporated herein by reference.

Specific examples of methods for the delivery of a butyrylcholinesterase variant by expressing the encoding nucleic acid sequence are well known in art and described in, for example, U.S. Pat. No. 5,399,346; U.S. Pat. Nos. 5,580,859; 5,589,466; 5,460,959; 5,656,465; 5,643,578; 5,620,896; 5,460,959; 5,506,125; European Patent Application No. EP 0 779 365 A2; PCT No. WO 97/10343; PCT No. WO 97/09441; PCT No. WO 97/10343, all of which are incorporated herein by reference. Other methods known to those skilled in the art also exist and are similarly applicable for the delivery of a butyrylcholinesterase variant by expressing the encoding nucleic acid sequence.

It is understood that modifications that do not substantially affect the activity of the various embodiments of this invention are also included within the definition of the invention provided herein. Accordingly, the following examples are intended to illustrate but not limit the present invention.

### EXAMPLE I

### Libraries of Butyrylcholinesterase Variants

This example describes the synthesis and characterization of butyrylcholinesterase variant libraries expressed in mammalian cells.

Initial libraries of butyrylcholinesterase variants were generated by mutating residues determined to be important for the catalytic activity of butyrylcholinesterase. Residues within butyrylcholinesterase that are potentially important for catalytic activity were determined by docking a substrate into the active site of butyrylcholinesterase with the FlexiDock program (Tripos Inc., St. Louis, Mo.) in Sybyl 6.4 software on a Silicone Graphics Octane computer. Residues important for catalytic activity were mutated using either PCR-based mutagenesis or codon-based mutagenesis as described herein and packaged into libraries.

Butyrylcholinesterase variant libraries were generated, for example, using PCR-site directed mutagenesis of human butyrylcholinesterase DNA performed utilizing Pfu polymerase (Stratagene, La Jolla, Calif.). Three oligonucleotide primers were used to perform the mutagenesis. The mutagenesis primers were used at the same time as a general primer such as the SP6 promoter sequencing primer (MBI Fermentas, Amherst, N.Y.) to amplify one end of the butyrylcholinesterase cDNA. The PCR reaction products (megaprimers) were cleaned on QuiaQuick PCR (Qiagen, Santa Clarita, Calif.) according to the manufacturer's protocol to remove excess primers. The cleaned megaprimers were extended in a second PCR reaction to generate the complete 1.8 kb coding sequence of each variant.

The 1.8-kb fragments constituting the butyrylcholinesterase variants were cloned into the plasmid pGS and resequenced to make sure the desired mutation was present. The plasmid pGS is identical with pRc/CMV (Invitrogen, Carlsbad, Calif.) except that the Neo gene has been replaced by rat glutamine synthesase. These variants can be stably expressed, for example, in Chinese Hamster Ovary (CHO) cell lines, or transiently expressed, for example, as described below in 293T cells.

Butyrylcholinesterase variant libraries were also generated, for example, using codon-based mutagenesis of human butyrylcholinesterase DNA. Regions of butyrylcholinesterase that were predicted to be important for catalytic efficiency based on structural modeling or sequence alignments between different species were targeted for mutagenesis. Seven regions that were predicted to be important for catalytic efficiency are shown in Table 1.

The seven regions of butyrylcholinesterase selected for focused library synthesis span residues that include the 8 aromatic active site gorge residues (W82, W112, Y128, W231, F329, Y332, W430 and Y440) as well as two of the catalytic triad residues. The integrity of intrachain disulfide bonds, located between ⁶⁵Cys-⁹²Cys, ²⁵²Cys⁻²⁶³Cys, and ⁴⁰⁰Cys⁻⁵¹⁹Cys is maintained to ensure functional butyrylcholinesterase structure. In addition, putative glycosylation sites (N-X-S/T) located at residues 17, 57, 106, 241, 256, 341, 455, 481, 485, and 486 also generally are avoided in the library synthesis. In total, the seven focused libraries span 79 residues, representing approximately 14% of the butyrylcholinesterase linear sequence, and result in the expression of about 1500 distinct butyrylcholinesterase variants.

Libraries of nucleic acids corresponding to the seven regions of human butyrylcholinesterase to be mutated are synthesized by codon-based mutagenesis, as described above. Briefly, multiple DNA synthesis columns are used for synthesizing the oligonucleotides by β-cyanoethyl phosphoramidite chemistry, as described previously by Glaser et al., supra, 1992. In the first step, trinucleotides encoding for the amino acids of butyrylcholinesterase are synthesized on one column while a second column is used to synthesize the trinucleotide NN(G/T), where N is a mixture of dA, dG, dC, and dT cyanoethyl phosphoramadites. Using the trinucleotide NN(G/T) results in thorough mutagenesis with minimal degeneracy, accomplished through the systematic expression of all twenty amino acids at every position.

Following the synthesis of the first codon, resins from the two columns are be mixed together, divided, and replaced in four columns. By adding additional synthesis columns for each codon and mixing the column resins pools of degenerate oligonucleotides will be segregated based on the extent of mutagenesis. The resin mixing aspect of codon-based mutagenesis makes the process rapid and cost-effective because it eliminates the need to synthesize multiple oligonucleotides. In the present study, the pool of oligonucleotides encoding single amino acid mutations are used to synthesize focused butyrylcholinesterase libraries. The oligonucleotides encoding the butyrylcholinesterase variants containing a single amino acid mutation can be cloned, for example, into the doublelox targeting vector using oligonucleotide-directed mutagenesis (Kunkel, supra, 1985).

Several butyrylcholinesterase variants from the libraries described above were found to have enhanced catalytic activity when compared to wild-type butyrylcholinesterase. Variants from the libraries described above were also assayed for enhanced carboxylesterase activity using a variety of assays described herein, such as the o-nitrophenyl acetate hydrolysis assay and HPLC assay for the formation of SN-38. One variant from these libraries, referred to as F227A (SEQ ID NO: 2), showed at least a 3 fold increase in butyrylcholinesterase activity compared to wild-type butyrylcholinesterase in the HPLC assay. F227A contains a single amino acid substitution in the human butyrylcholinesterase polypeptide sequence which replaces a phenylalanine at position 227 with an alanine. At the DNA level this is a change from a TTT codon to a GCG codon.

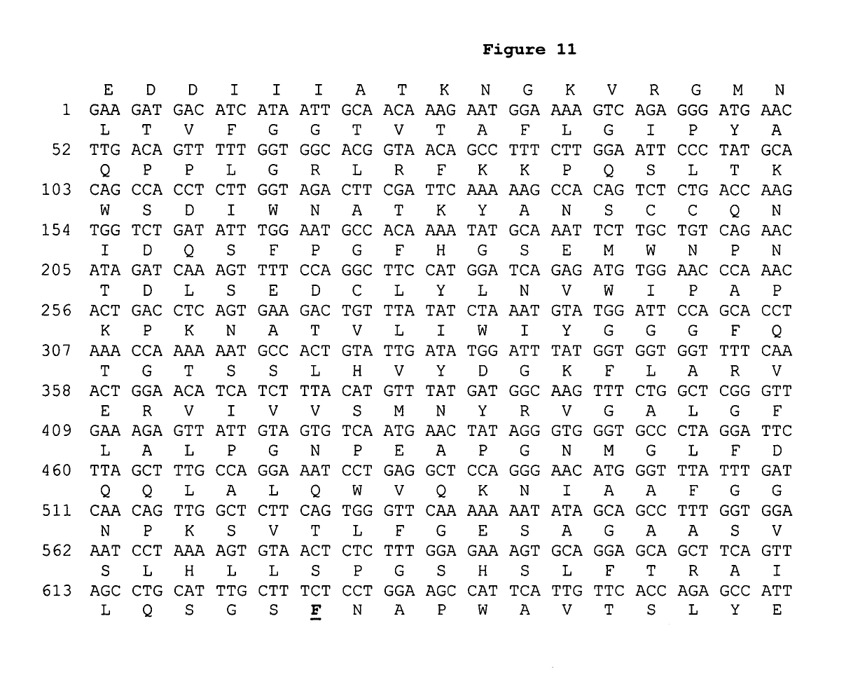

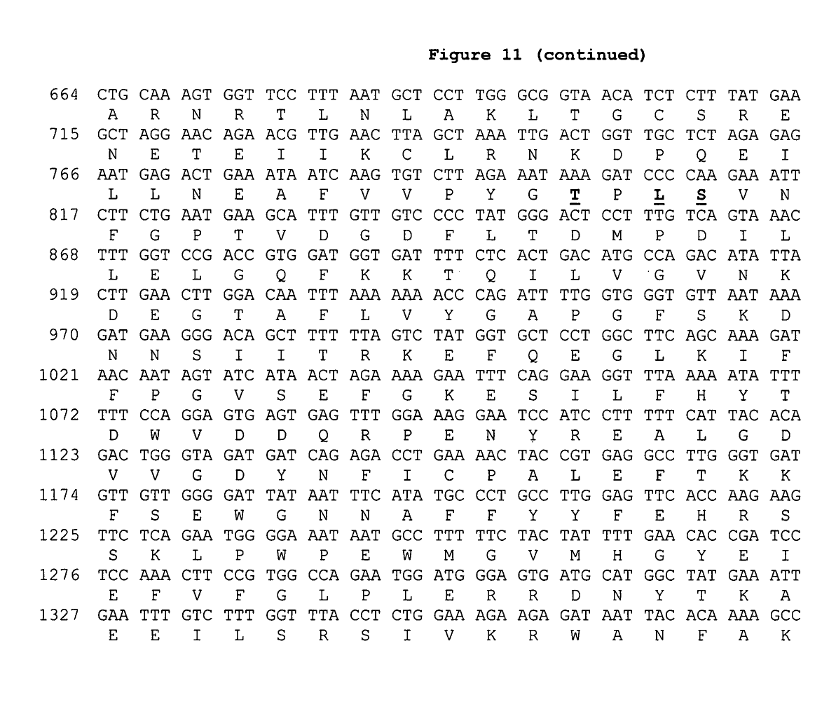

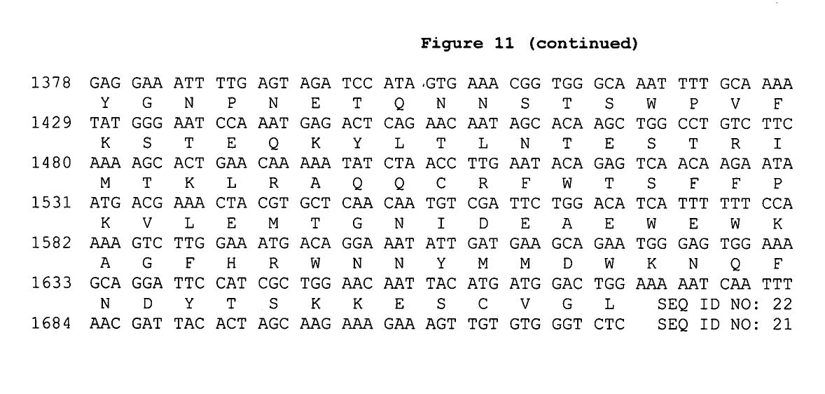

Using the F227A variant as a template, additional site-directed mutations were generated resulting in the construction of several double mutants. The regions chosen for site-directed mutation were residues predicted to be important for catalytic efficiency as described herein (see, for example, Table 1). For example, the following double mutants were generated (see Table 2). The nucleotide and amino acid sequence of human butyrylcholinesterase (SEQ ID NOS: 21 and 22) is shown in FIG. 11 for reference.

TABLE 2

| | Amino acid change | Codon change |
|---|---|---|
| | (in addition to F227A change, | (in addition to F227A |
| BChE Variant | phenylalanine to alanine) | change, TTT to GCT) |
| F227A/T284A | threonine to alanine | ACT to GCT |
| F227A/L286Q | leucine to glutamine | TTG to CAG |
| F227A/L286S | leucine to serine | TTG to TCG |
| F227A/L286H | leucine to histidine | TTG to CAT |
| F227A/L286W | leu to tryptophan | TTG to TGG |
| F227A/S287P | serine to proline | TCA to CCT |

Butyrylcholinesterase variants that contain double mutations were expressed in a transient system using 293T human embryonic kidney cells. Briefly, on day 1, 293T cells were plated at 1.5×10⁵ cells/well in a BioCoat 24-well plate. The cells were then allowed to recover overnight. On the second day, dilute 2 ul of Lipofectamine 2000/well in 50 ul Opti-MEM/well, and incubate 5 minutes. Dilute 500 ng-1 ug DNA/well in 50 ul Opti-MEM/well. The two diluted solutions were mixed, and incubated for 20 minutes at room temperature. Media was removed from cells and replaced with 500 ul/well complete growth media, without penicillin or streptomycin. Subsequently, 100 ul of diluted solutions was added to each well, and incubated on cells for 4 hours. The media/DNA/Lipofectamine 2000 was removed from cells, and replaced with 1 ml of Ultraculture serum free media (Bio Wittaker) per well. The butyrylcholinesterase variant polypeptides were allowed to accumulate for 48-96 hours and the conditioned media from the cells was used directly. For other applications the butyrylcholinesterase variant polypeptides can be purified as described below in Example VI.

Butyrylcholinesterase variants that contain double mutations were assayed for activity as described below. For example, the variants were assayed for carboxylesterase activity using an o-nitrophenyl acetate hydrolysis assay, CPT-11 conversion activity using an HPLC based assay, and cytotoxicity of a cancer cell line.

### EXAMPLE II

### Carboxylesterase Activity of Butyrylcholinesterase Variants

This example shows carboxylesterase activity of several butyrylcholinesterase variants.

A standard assay for carboxylesterase activity is the o-nitrophenyl acetate (o-NPA) hydrolysis assay (see Beaufay et al., J. Cell. Biol. 61: 188-200 (1974)). A modification of this assay that measures o-NPA activity of butyrylcholinesterase variants normalized by capturing with an anti-butyrylcholinesterase antibody was performed as described below.

Protocol to Determine Carboxylesterase activity of Captured Butyrylcholinesterase by o-Nitrophenyl acetate:
1) Coat 96-well Immulon 2 plates with rabbit anti-human butyrylcholinesterase (Dako #A0032) at 10 mg/ml in PBS (100 ml/well) overnight at 4° C.
2) Remove coating solution and block plate with 3% BSA in PBS (250 ml/well) for 2 hours at room temperature.
3) Add 200 ml butyrylcholinesterase variant conditioned media and incubate at RT for 2 hrs.
4) Wash plate 3 times with 250 ml/well PBS.
5) Add 85 ml/well 0.1 M potassium phosphate pH 7.0
6) Add 13.6 mg o-NPA to 100 ml acetonitrile, mix to dissolve. Add 100 ml of this stock to 6.3 mls water and mix well.
7) Wash plate 3 times with PBS.
8) Add 15 ml of diluted o-NPA substrate.
9) Read absorbance at 405 nm.

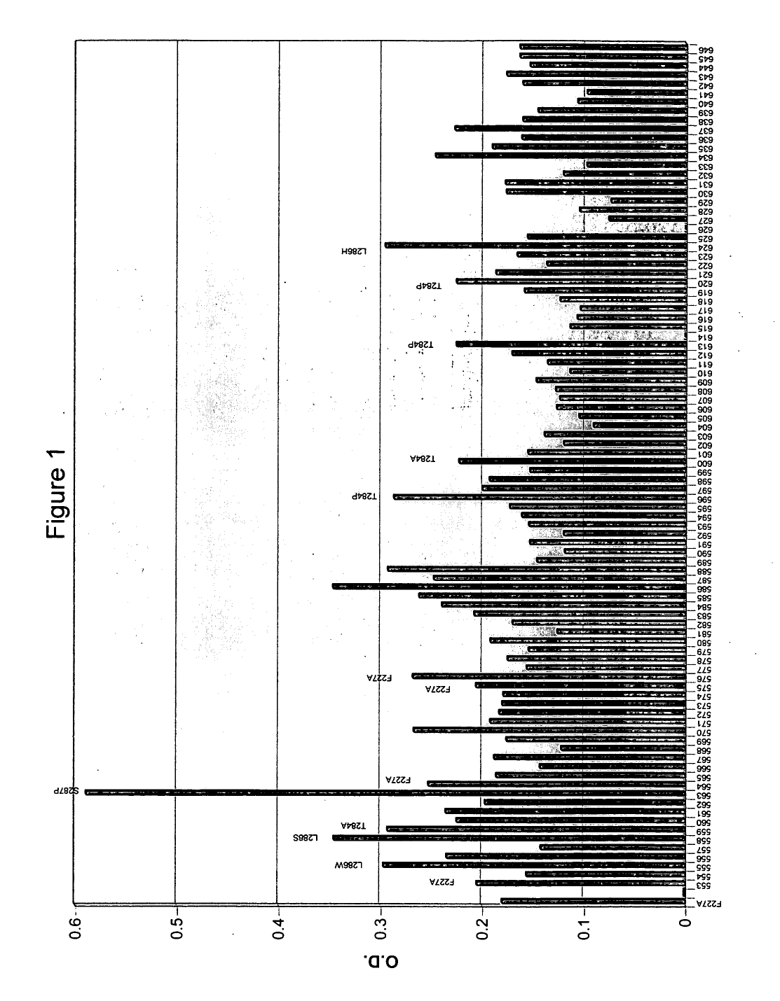

Conditioned media from butyrylcholinesterase variants that were transiently expressed in 293T cells was tested using the above described anti-butyrylcholinesterase antibody capture/normalization assay for carboxylesterase activity. As shown in the representative assay in FIG. 1, several butyrylcholinesterase variants showed carboxylesterase activity. The first variant shown in FIG. 1 is F227A (see left-most bar). The level of activity of the other variants can be compared to the level of activity of F227A. The level of activity of wild-type butyrylcholinesterase in this assay was approximately 50% of the level of activity seen with F227A. In order to determine the amount of variability present in the assay, several wells contained the same variant. For example, four wells are labeled as containing the F227A variant in addition to the first well. The level of activity in all five wells is similar and demonstrates a low level of variability within this assay.

Butyrylcholinesterase residues identified by this method that effect carboxylesterase substrate hydrolysis include the following: F227, A328, Y332, T284, P285, L286. This assay can be used to quickly screen a number of variants for activity. Activity measured in this assay can be predictive of CPT-11 activation and can be used to identify residues or regions of BChE involved in CPT-11 activation.

### EXAMPLE III

### CPT-11 Conversion Activity of Butyrylcholinesterase Variants

This example shows butyrylcholinesterase variants that have increased CPT-11 conversion activity compared to butyrylcholinesterase.

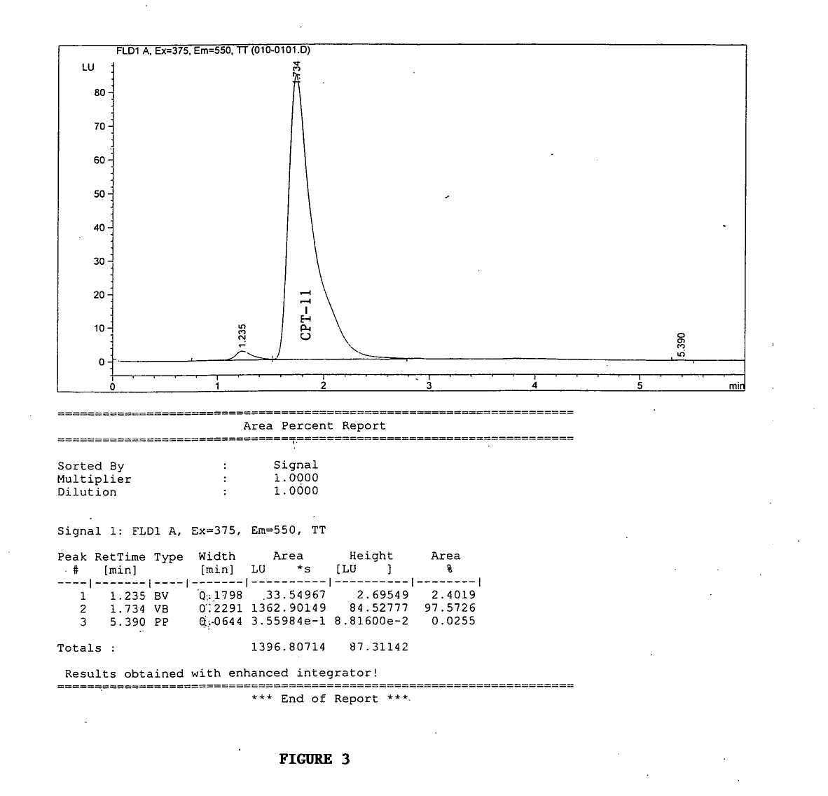

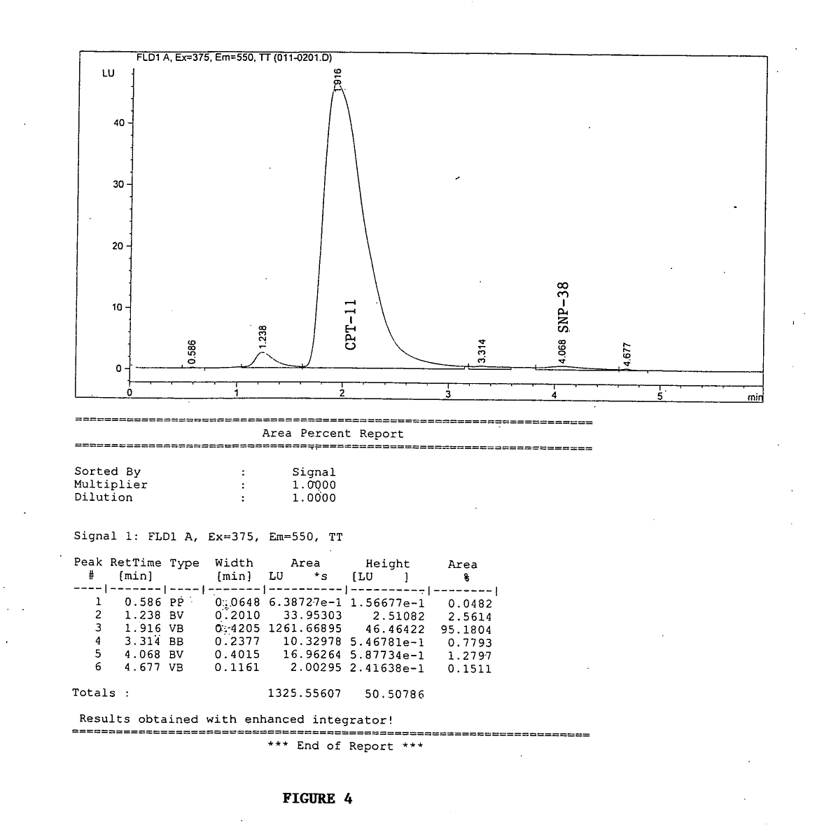

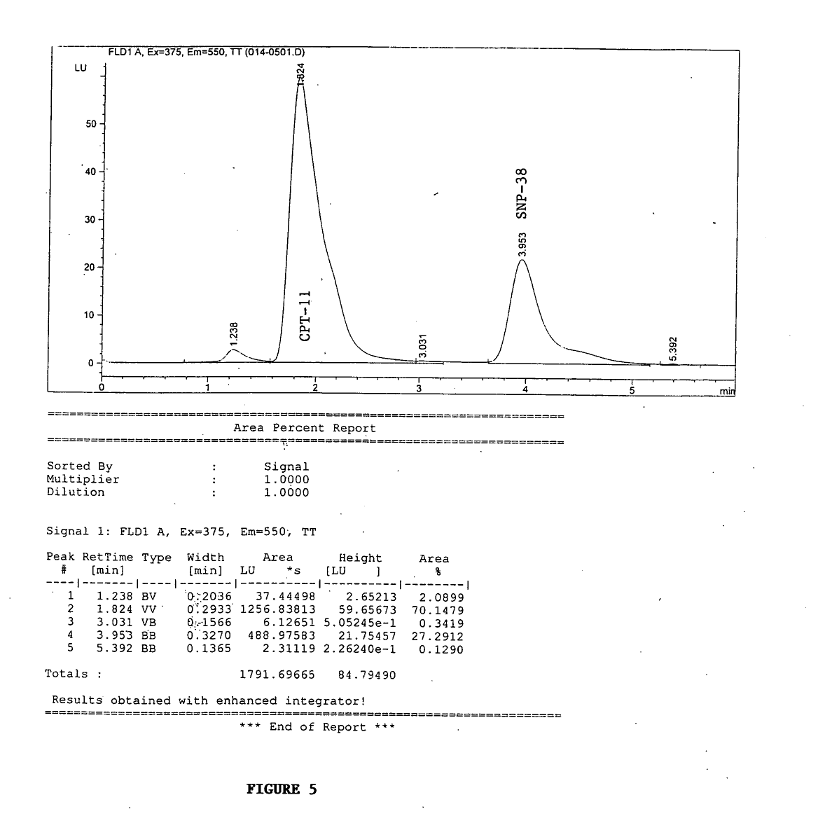

Butyrylcholinesterase variants were assayed for CPT-11 conversion activity using fluorescent High Performance Liquid Chromotography (HPLC) detection of SN-38 formation as described in Dodds and Rivory, Mol. Pharmacol. 56: 1346-1353 (1999), which is incorporated herein by reference. The conversion of CPT-11 to SN-38 is shown in FIG. 2. Briefly, conditioned media from transiently expressed BChE variants were exposed to 20 mM CPT-11 for 72 hours at 37° C. and analyzed by HPLC for SN-38 formation (peak at about 4 minutes column retention time). FIG. 3 shows the amount of SN-38 produced using conditioned media from cells that were mock-transfected which means that the transfection was performed as usual however no DNA was added. FIG. 4 shows the amount of SN-38 produced using conditioned media from cells that were transfected with F227A, and FIG. 5 shows the amount of SN-38 produced using conditioned media from cells that were transfected with F227A/L286S.

As shown in FIGS. 3-5, the F227A variant produced a small amount of SN-38 and variant F227A/L286S showed significant conversion Of CPT-11 to SN-38 as compared to mock and F227A variant conditioned medias. In this assay, wild-type butyrylcholinesterase did not show detectable levels of SN-38.

Butyrylcholinesterase variants identified by this method that had increased CPT-11 conversion to SN-38 compared to wild-type butyrylcholinesterase include: F227A (SEQ ID NO: 2), F227A/T284A (SEQ ID NO: 4), F227A/L286Q (SEQ ID NO: 6), F227A/L286S (SEQ ID NO: 8), F227A/L286H (SEQ ID NO: 10), F227A/L286W (SEQ ID NO: 12), and F227A/S287P (SEQ ID NO: 14). Table 3 shows the approximate fold improvement in CPT-11 conversion to SN-38 in these variants. The actual fold improvement can be significantly higher than listed in Table 3 because the values in Table 3 are based on the fold improvement compared to wild-type butyrylcholinesterase. Since the activity of wild-type butyrylcholinesterase is extremely low in this assay (less than 1% conversion), the value for wild-type butyrylcholinesterase is prone to more variability than other activity values. Therefore, the activity values listed in Table 3 are very conservative values for the activity of these variants and so the variants in Table 3 have at least the listed activity value and more. For example, the butyrylcholinesterase variant F227A/L268S (SEQ ID NO: 8) has at least a 50-fold increase in camptothecin conversion activity compared to butyrylcholinesterase. Said another way, the butyrylcholinesterase variant F227A/L268S (SEQ ID NO: 8) exhibits a 50-fold or greater increase in conversion capability compared to the butyrylcholinesterase.

TABLE 3

| | | Fold improvement in CPT-11 |
|---|---|---|
| SEQ ID NO: | BChE Variant | conversion |
| 2 | F227A | >3 |
| 4 | F227A/T284A | >7 |
| 6 | F227A/L286Q | >10 |
| 8 | F227A/L286S | >50 |
| 10 | F227A/L286H | >35 |
| 12 | F227A/L286W | >42 |
| 14 | F227A/S287P | >6 |

### EXAMPLE IV

### Butyrylcholinesterase-Mediated Cytotoxicity and Enchanced CPT-11 Activated Killing by Butyrylcholinesterase Variants

This example shows butyrylcholinesterase variants that have increased cytotoxicity in a cancer cell line compared to butyrylcholinesterase.

A cellular cytotoxicity assay was used to demonstrate the level of CPT-11 activation by BChE variants. Clinically relevant concentrations of CPT-11 (0.5-10 mM) were exposed to BChE variants for 24-72 hours at 37° C. Briefly, CPT-11 at 4 mM was incubated for 72 hours with expressed wild-type BChE, the 6-6 variant, or F227A/L286Q variant. SW48 colon carcinoma cells were exposed to the activated CPT-11 at a concentration of 0.5 mM for 72 hours and cell viability measured by the MTT method. The MTT (3-(4,5-dimethylthiazolyl-2)-2,5-diphenyltetrazolium bromide) assay is commercially available and can be used to measure cell viability based on the ability of a cell to reduce a redox sensitive dye. Note the 6-6 variant is a quadruple mutant referenced as A328W/Y332M/S287G/F227A (SEQ ID NO: 16) in the nomenclature used throughout to describe variants. The 6-6 variant contains the following codons at variant positions: GCG encodes alanine at amino acid position 227, GGT encodes glycine at amino acid position 287, ATG encodes methionine at amino acid position 332, and TGG encodes tryptophane at amino acid position 328.

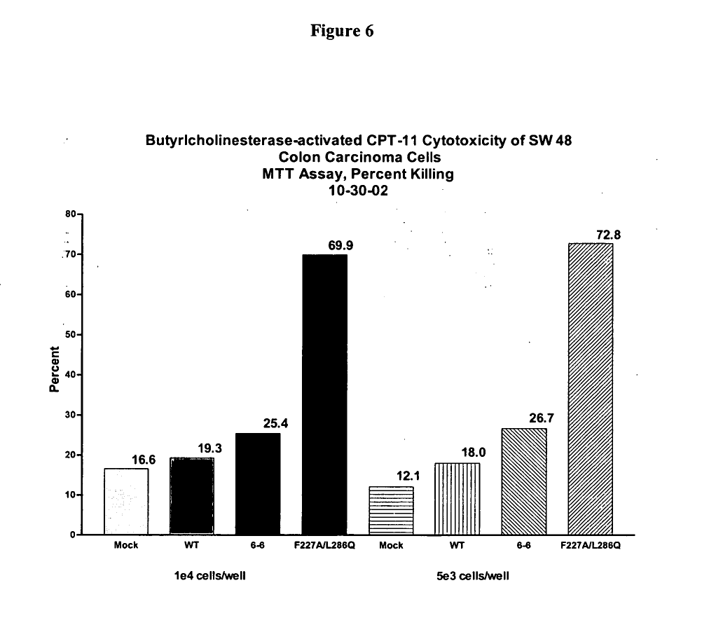

As shown in FIG. 6, CPT-11 mediated cytotoxicity in SW48 colon carcinoma cells is significantly enhanced in the presence of BChE and BChE variants. Both the 6-6 variant (SEQ ID NO: 16) and F227/L286Q (SEQ ID NO: 6) variant showed increased tumor cell cytotoxicity mediated by CPT-11 activation.

### EXAMPLE V

### Antibody-Butyrylcholinesterase Fusion Polypeptide for the Targeted Activation of CPT-11

This example shows the construction and characterization of an anti-EGF receptor-BChE fusion polypeptide which can be used for Antibody Directed Enzyme Pro-drug Therapy (ADEPT) with CPT-11.

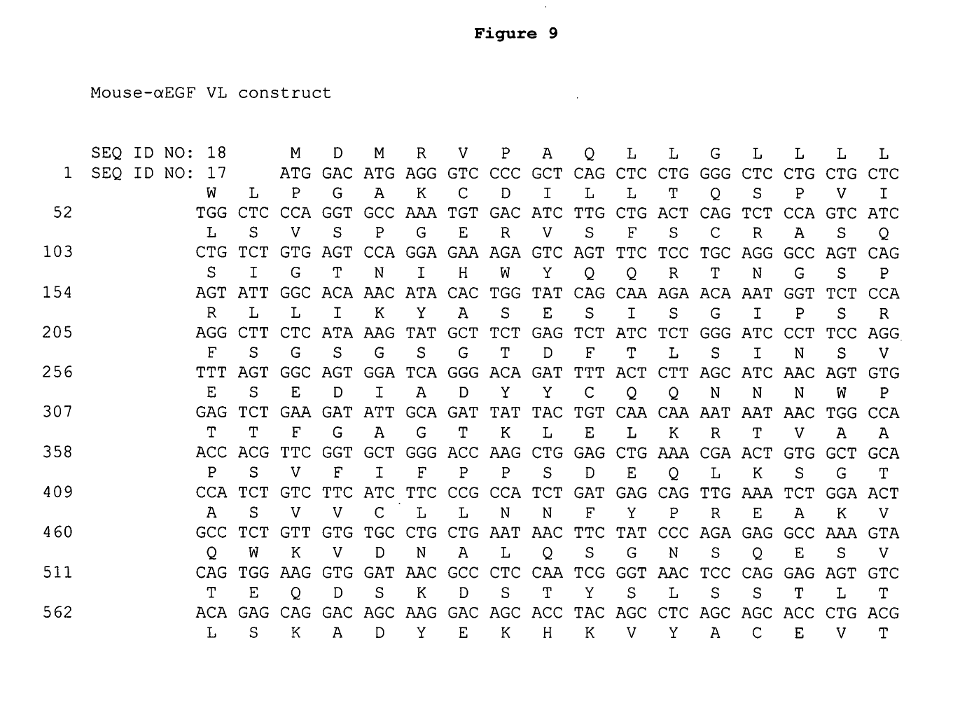

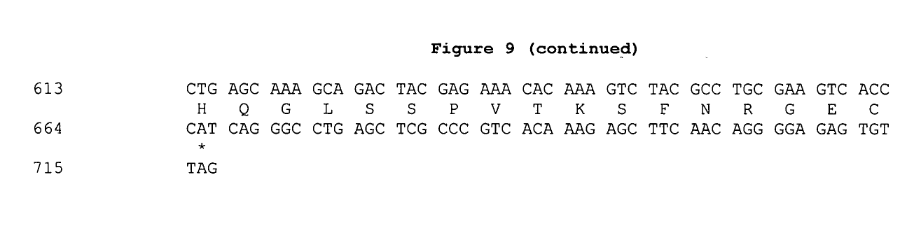

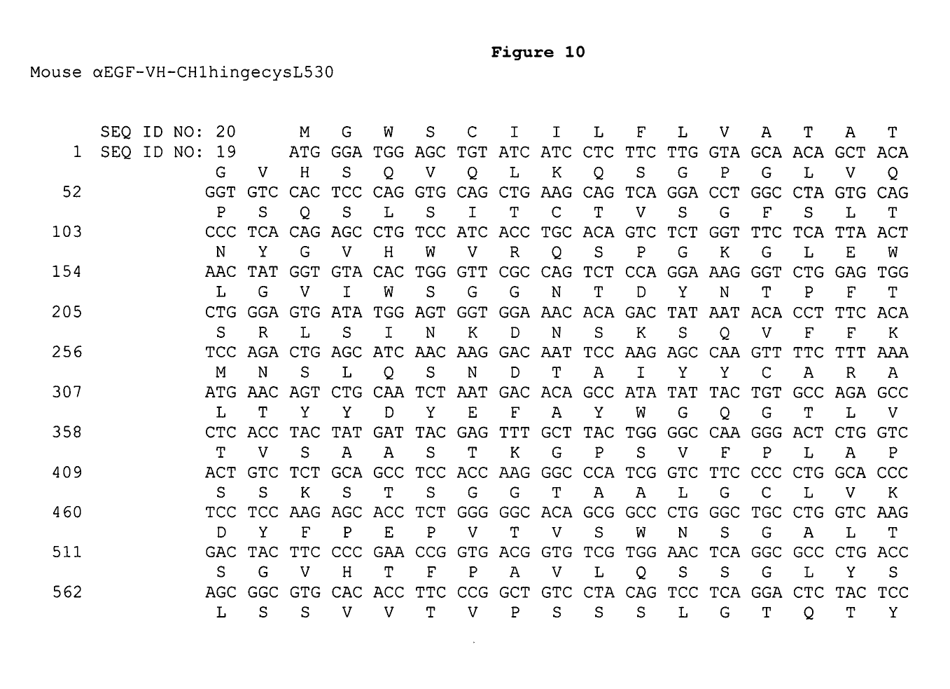

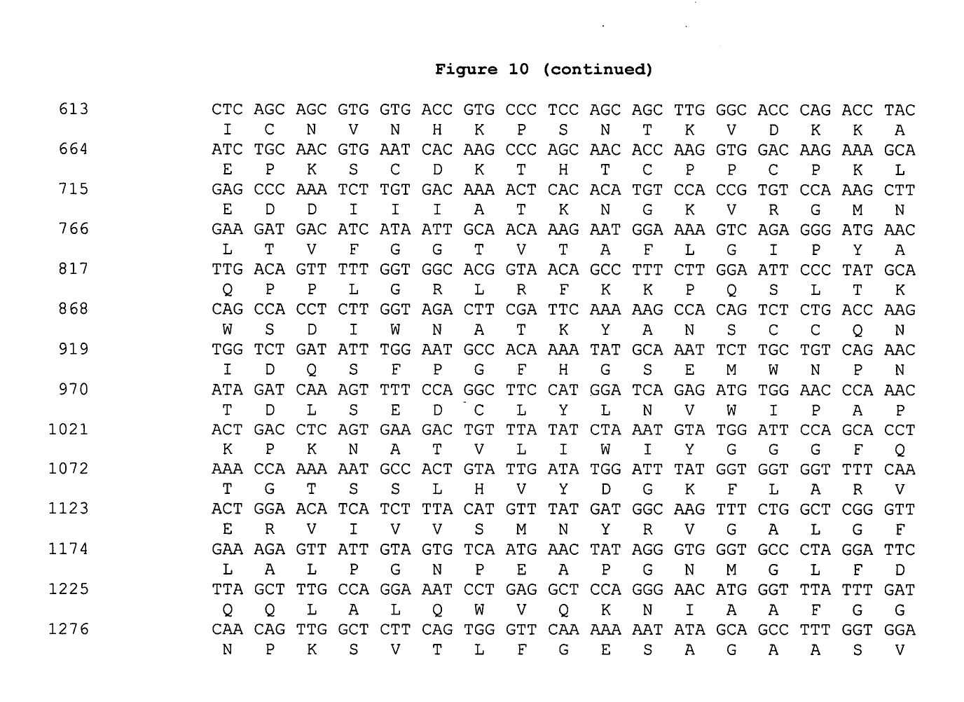

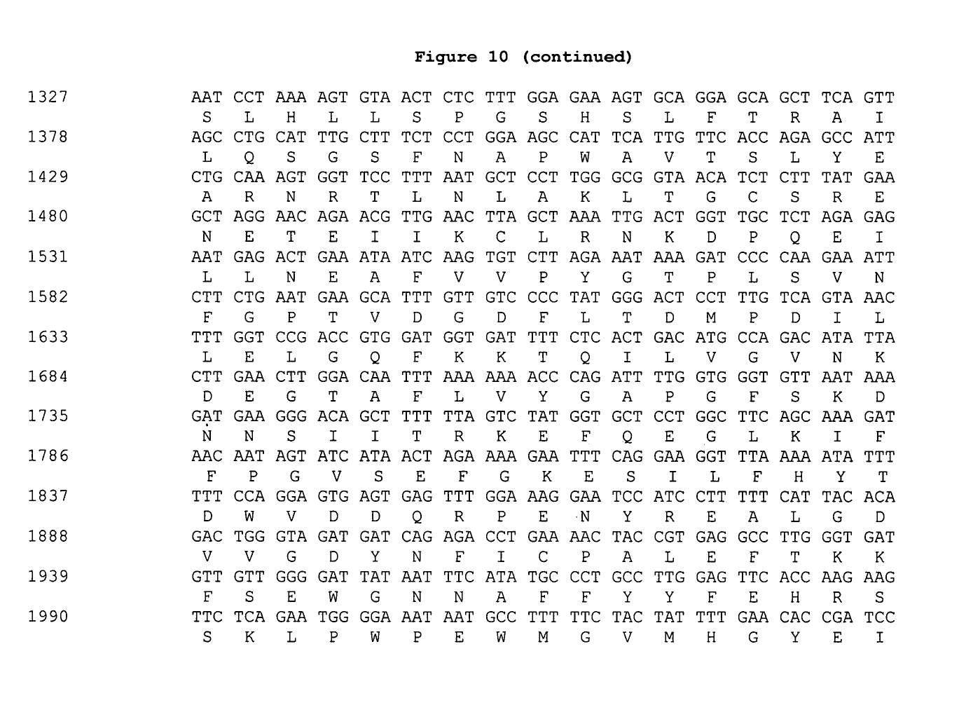

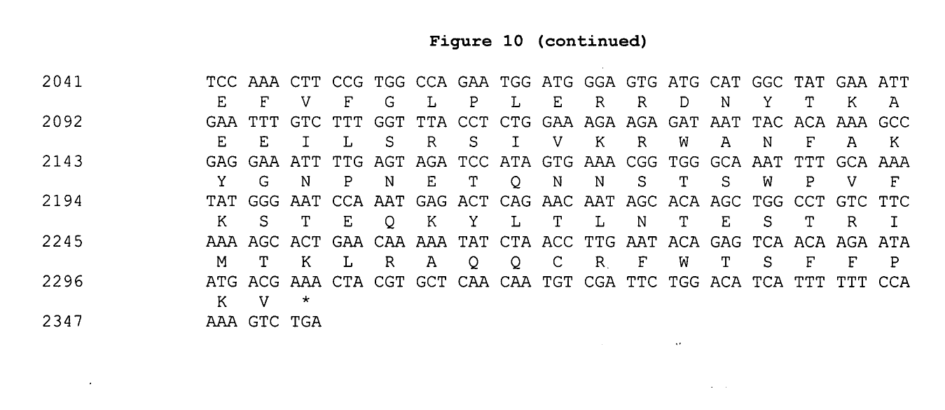

A model antibody-BChE fusion polypeptide was constructed by fusing the N-terminus of the BChE (truncated L530 monomer) to the C-terminus of the CH1 domain of the anti-epidermal growth factor receptor (EGFR) antibody ch225. The two domains are linked by either a GGGS linker or the natural antibody hinge region. A model antibody-enzyme fusion polypeptide was produced which exhibits both antigen binding and catalytic enzyme functions. The nucleotide and amino acid sequence of the mouse anti-EGF variable light chain is shown in FIG. 9 and the nucleotide and amino acid sequence of the mouse anti-EGF variable heavy chain and constant heavy chain 1 hinge region of L530 is shown in FIG. 10.

The truncated L530 monomer is described in Blong et al., Biochem. J. 327: 747-757 (1997) and is incorporated herein by reference. This monomer contains a BChE which is truncated at the C terminus such that it does not assemble into a tetramer as seen with wild-type BChE. The L530 monomer does retain most or all butyrylcholinesterase (BChE) activity.

An ELISA assay showing binding of expressed anti-EGFR-BChE L530 to anti-kappa capture antibody and measuring activity of bound butyrylcholinesterase by butyrylthiocholine hydrolysis is shown in FIG. 7. This demonstrates that intact fusion polypeptide (bound through the antibody light chain) exhibits butyrylcholinesterase activity.

The protocol for anti-Kappa capture of anti-EGFR-L530 is as follows:
1) Coat 96-well Immulon 2 plates with 200 ml of 10 mg/ml anti-human Kappa antibody in PBS overnight.
2) Block plate with 3% BSA in PBS (250 ml/well) for 2 hours at room temperature.
3) Wash plate 3× with 250 ml/well PBS.
4) Add 200 ml BChE conditioned media and incubate at RT for 2 hours.
5) Prepare working solution of DTNB by making a 1:10 dilution of stock 5 mM DTNB in 0.1 M Potassium phosphate pH 7.0. Add 180 ml per well
6) Prepare working solution of Butyrythiocholine (BTC) by making 1:20 dilution of 200 mM stock in water. Add 20 ml per well.
7) Incubate plate at 37° C. and read on a spectrophotometer at A405 nm.

An ELISA assay measuring butyrylcholinesterase activity of the anti-EGFR-BChE L530 specifically bound to a cell membrane preparation containing the EGFR antigen is shown in FIG. 8. These results demonstrates antigen-specific binding of the fusion protein through the antibody domain and enzymatic activity of the butyrylcholinesterase domain.

The protocol for anti-EGFR binding to A431 membrane preparations is as follows:
1) Coat 96-well Immulon 2 plates with 50 ml/well of A431 cell lysate diluted 1/20 in 10 mM HEPES pH 7.4, 0.1% Triton X-100 and dry in the hood overnight.
2) Block plate with 3% BSA in PBS (250 ml/well) for 2 hours at room temperature.
3) Wash plate 3× with 250 ml/well PBS.
4) Add 200 ml BChE conditioned media and incubate at RT for 2 hours.
5) Prepare working solution of DTNB by making a 1:10 dilution of stock 5 mM DTNB in 0.1 M Potassium phosphate pH 7.0. Add 180 ml per well
6) Prepare working solution of Butyrythiocholine (BTC) by making 1:20 dilution of 200 mM stock in water. Add 20 ml per well.
7) Incubate plate at 37° C. and read on a spectrophotometer at A405 mm.

### EXAMPLE VI

### Purification and Characterization of the Butyrylcholinesterase Variants

This example shows how butyrylcholinesterase variant polypeptides can be purified. These purified polypeptides can be used, for example, in the assays and pharmaceutical compositions described herein.

To purify the butyrylcholinesterase variants, the culture medium corresponding to each variant is filtered through Whatman #1 filter paper (Whatman Inc., Clifton, N.J.) on a Buchner funnel. The filtrate is poured through a chromatography column (XK50/30, Pharmacia Biotech, Piscatawy, N.J.) packed with 100 ml of affinity gel procainamide-Sepharose 4B. The butyrylcholinesterase variants stick to the affinity gel during loading so that 20 mg of enzyme that was previously in 20 liters is concentrated in 100 ml of affinity gel. The affinity gel is subsequently washed with 0.3M sodium chloride in 20 mM potassium phosphate pH 7.0 and 1 mM EDTA to elute contaminating proteins. Next, the affinity gel is washed with buffer containing 20 mM potassium phosphate and 1 mM EDTA pH 7.0 to reduce the ionic strength. Finally, the butyrylcholinesterasae variants is eluted with 250 ml of 0.2M procainamide in buffer.

To further purify the butyrylcholinesterase variants and remove the procainamide a second purification step can be performed. The butyrylcholinesterase variants recovered in the first purification step are diluted 10-fold with buffer (20 mM TrisCl, 1 mM EDTA pH 7.4) to reduce the ionic strength to about 0.02M. The diluted enzyme is loaded onto a column containing 400 ml of the weak anion exchanger DE52 (Whatman, Clifton, N.J.). At this low ionic strength the butyrylcholinesterase variant sticks to the ion exchange gel. After loading is complete the column is washed with 2 liters of buffer containing 20 mM TrisCl and 1 mM EDTA pH7.4 until the absorbency of the eluant at 280 nm is nearly zero, indicating that the procainamide has washed off. Subsequently, the butyrylcholinesterase variants are eluted from the column with a salt gradient from 0 to 0.2M NaCl in 20 mM TrisCl pH 7.4. Following the elution of the butyrylcholinesterase variants 10 ml fractions are collected for each variant using a fraction collector. Activity assays are performed to identify the peak containing butyrylcholinesterase variant. SDS gel electrophoresis can be performed to determine the purity of each butyrylcholinesterase variants, which is typically determined to be approximately 90%.

### EXAMPLE VII

### Production of Antibody-BChE Fusion Proteins and Evaluation of Targeted Prodrug Activation and Cytotoxicity In Vitro

This example demonstrates optimization BChE variants in an antibody directed enzyme prodrug therapy model. Antibody-enzyme fusion proteins incorporating optimized variant residues were identified by library screening and the feasibility of using optimized BChE in ADEPT with CPT-11 was confirmed by targeting CD20, a non-internalizing cell surface antigen present on B-lymphocytes.

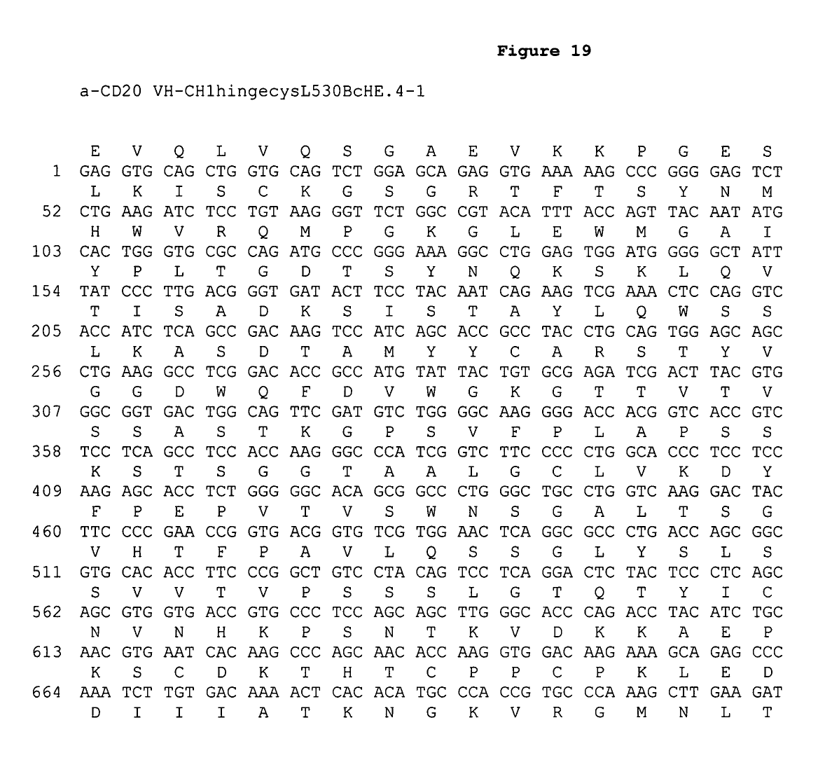

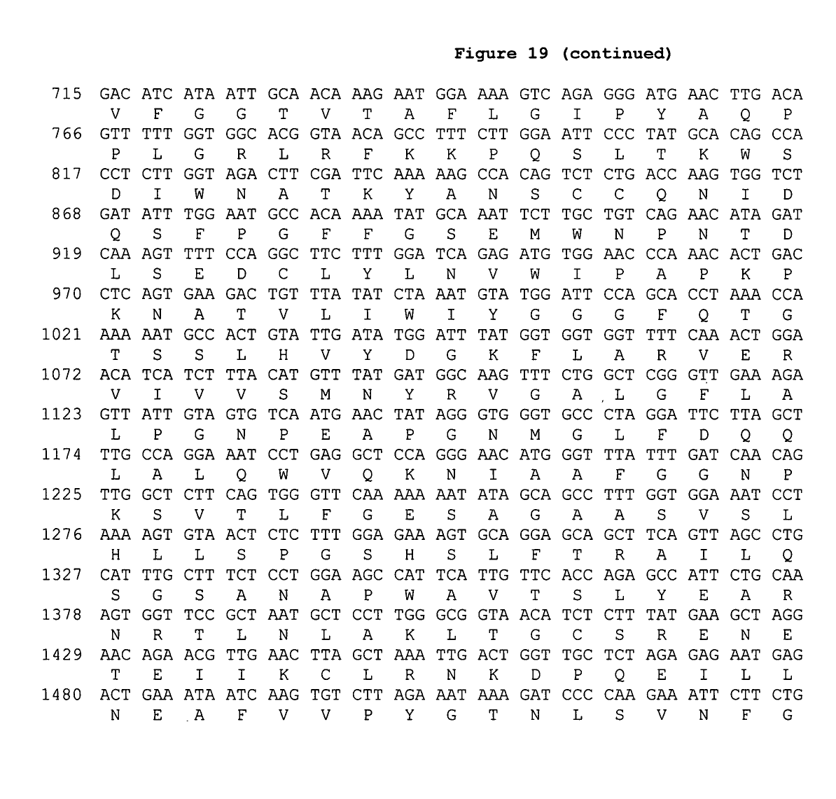

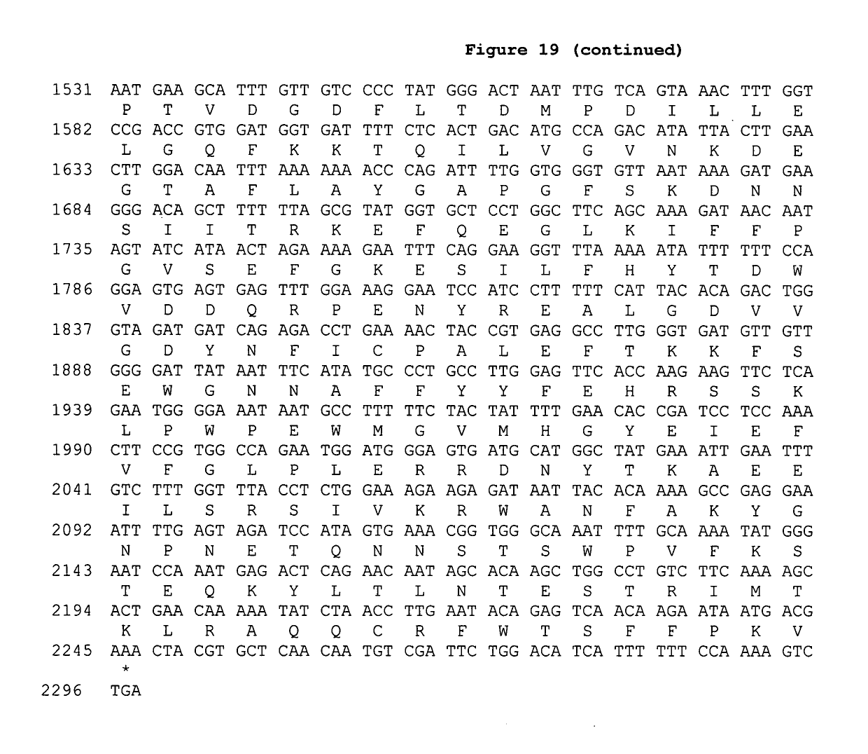

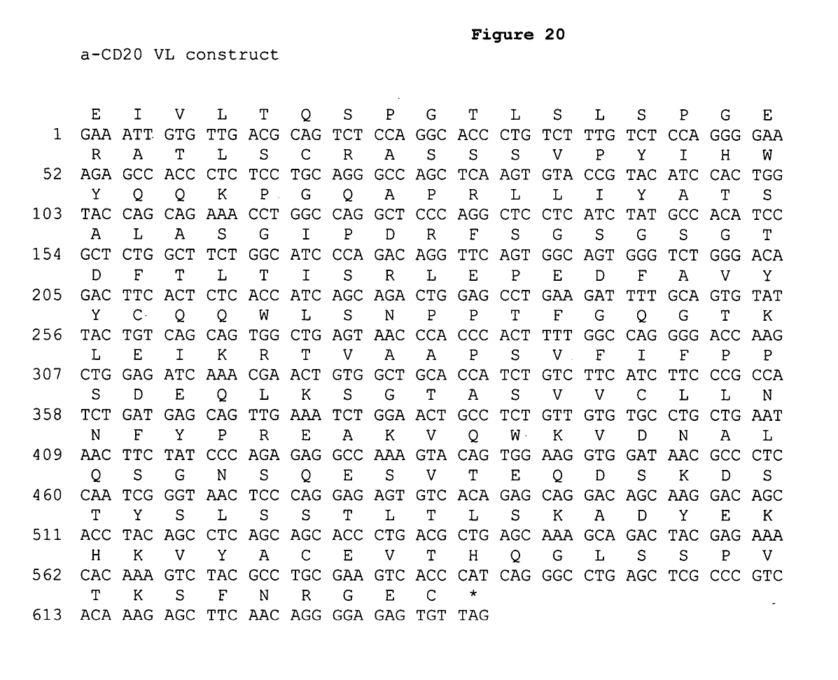

CD20 is a useful target antigen to test the feasibility of using optimized BChE in ADEPT as the antigen is abundantly expressed on human B lymphoma lines (3.2×10⁵ molecules/cell) and does not undergo significant internalization upon antibody binding. AME133 is a humanized anti-CD20 with fully human germline framework regions shown in FIGS. 19 and 20 and set forth as SEQ ID NOS: 198 and 200, with corresponding nucleic acid sequences SEQ ID NOS: 197 and 199. This antibody was generated at Applied Molecular Evolution using the company's directed evolution strategies, and the monovalent Fab binds to CD20 with extremely high affinity (1×10⁻⁹ M). An exemplary model fusion protein (anti-CD20-BChE.4-1) was generated and is shown in FIG. 19. Anti-CD20-BChE.4-1 (SEQ ID NO: 202) is composed of AME133 Fab fused at the C-terminal end of the CH1 heavy chain domain to the N-terminus of modified BChE variant L530 (SEQ ID NO: 204), which is a functional fragment of reference SEQ ID NO: 180. The BChE variant was truncated at amino acid 530 to abrogate its normal assembly into tetramers. The monomeric version of the enzyme exhibits the equivalent activity of each subunit of the naturally occurring tetrameric form, with no loss of activity due to allosteric effects as described by Blong et al., Biochem J 327 (Pt 3): 747-57 (1997).

Non-adherent HEK 293 cells were adapted to suspension culture in serum-free low protein media (UltraCULTURE, BioWhittaker) and transiently transfected with fusion protein, producing yields of 2-4 mg/L. A two-step purification procedure was developed using anion exchange on Sepharose Q followed by a hydrophobic interaction chromatography step on phenyl Sepharose. This resulted in product of >90% purity with one major contaminant band detected by SDS-PAGE. The fusion protein maintains a Km value for CPT-11 identical to that of the BChE.4-1 variant alone. Control fusion protein constructs have also been generated and expressed. These are AME133 Fab fused at the C-terminal end of the CH1 heavy chain domain to the N-terminus of truncated wild type BChE (anti-CD20-BChE.wt) and anti-epidermal growth factor receptor (EGFR) Fab fused to BChE.4-1.

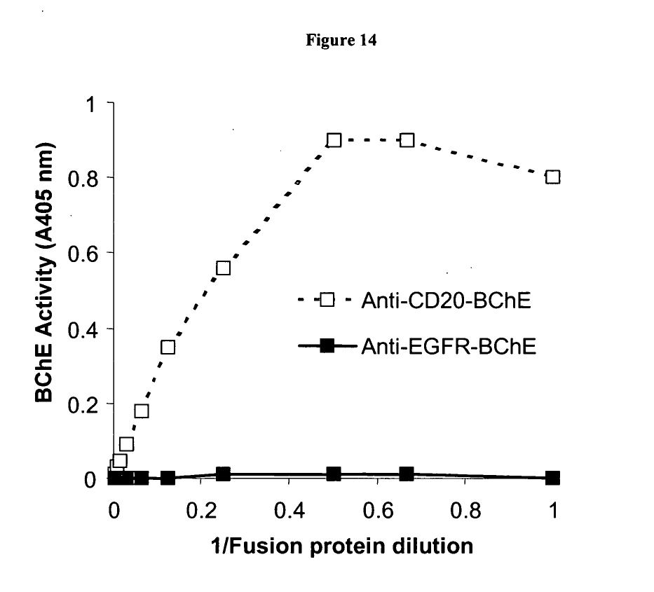

As shown in FIG. 14, anti-CD20-BChE fusion proteins exhibit antigen specific binding to CD20 positive SKW tumor cells as compared to the control anti-EGFR-BChE construct. Bound fusion protein was detected by BChE specific hydrolysis of butyrylthiocholine iodide.

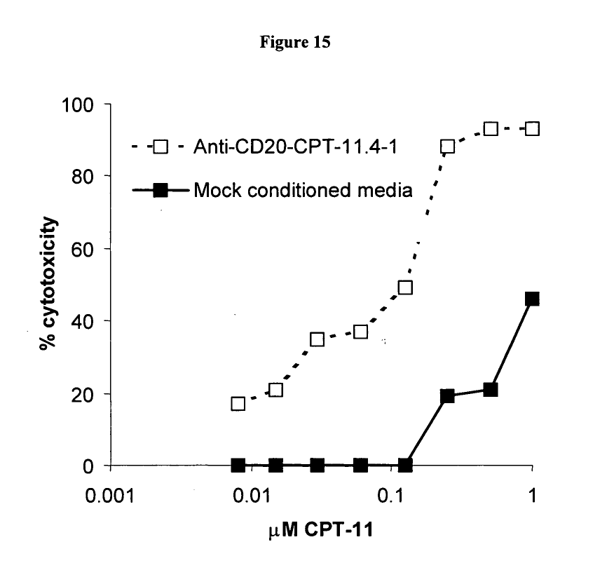

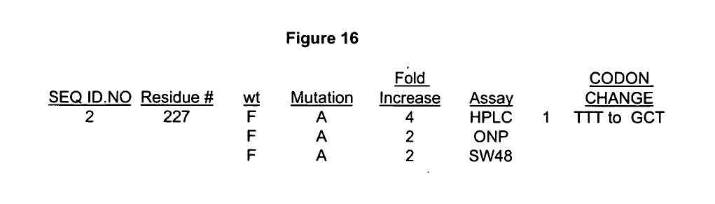

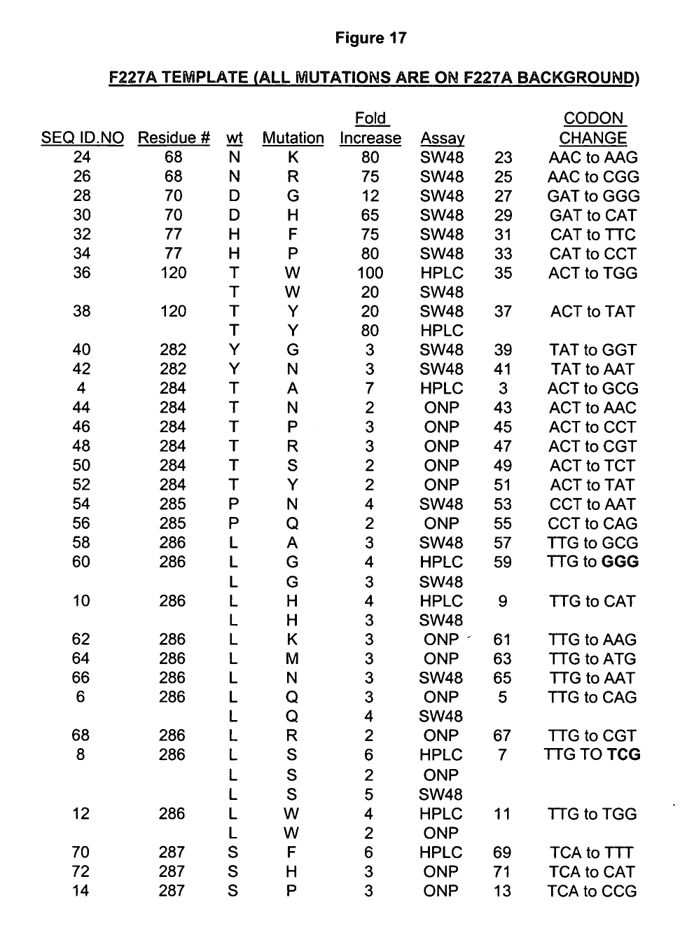

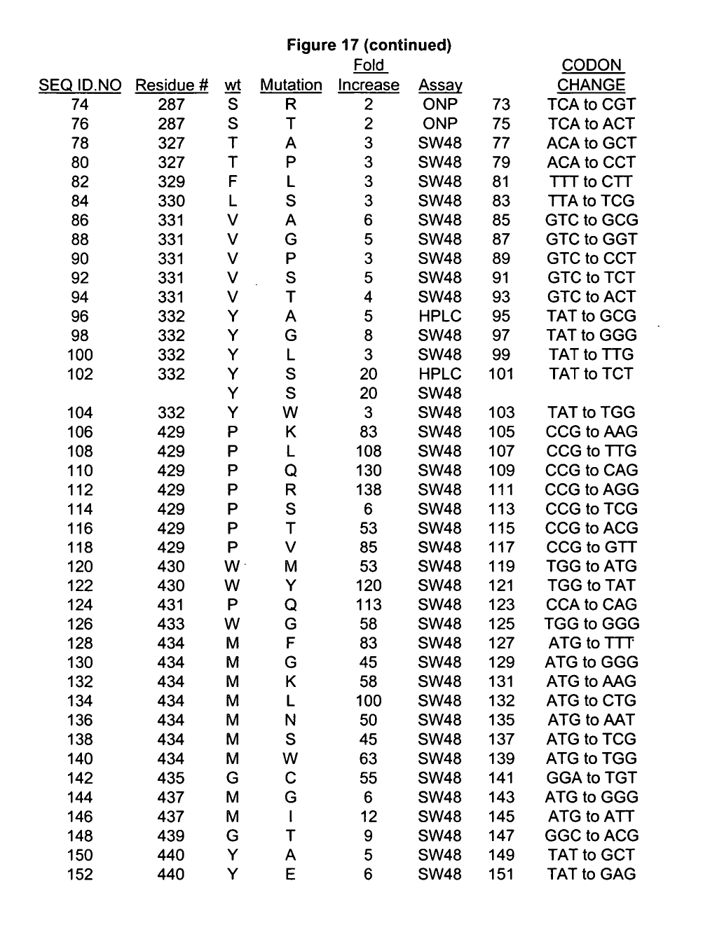

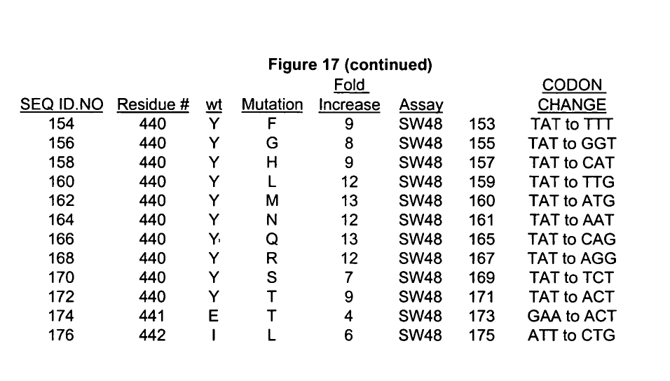

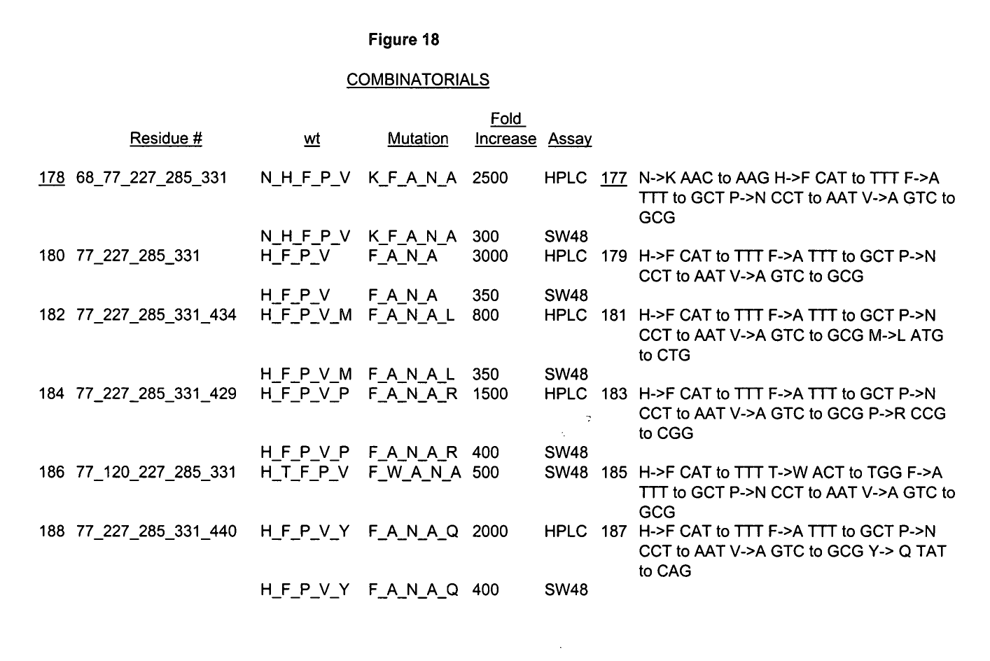

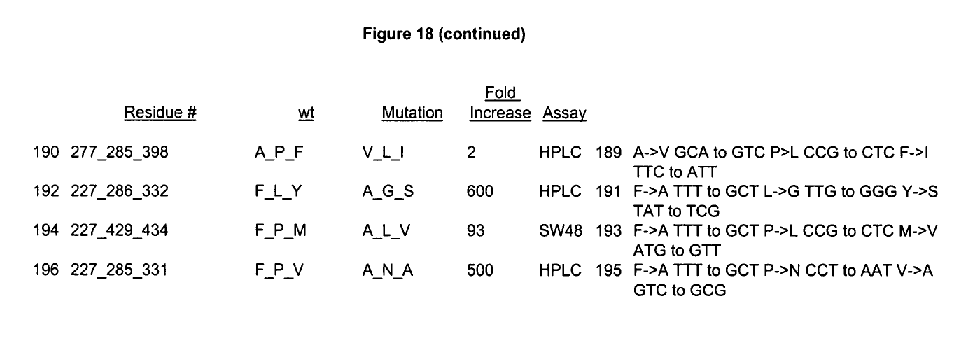

In order to demonstrate the utility of the fusion protein in ADEPT, it was shown that the anti-CD20-BChE fusion protein binds tightly to tumor cells in vitro and generates cytotoxic SN38 in the presence of CPT-11. Antigen-positive SKW 6.4 human B lymphoma cells were incubated with anti-CD20-BChE.4-1 and unbound fusion protein was removed by washing. Cells were then incubated with increasing concentrations of CPT-11 for four hours and washed again. Cell viability was assessed 72 hours later. Following incubation with anti-CD20-BChE.4-1, a 7 to 10-fold decrease in the EC₅₀ of CPT-11 was observed, relative to cells treated with mock conditioned media (FIG. 15). Briefly, live tumor cells were pre-incubated with anti-CD20-BChE.4-1 or mock conditioned media and washed prior to 4-hour exposure to CPT-11 over a range of concentrations. Bound fusion protein resulted in a marked decrease in the EC50 for CPT-11 killing. Cell viability was evaluated in a 72 hour MTT assay. Similar experiments using control anti-EGFR-BChE.4-1 or anti-CD20-BChE.wt showed no increase in cytotoxicity over CPT-11 alone. In these experiments approximately 0.8 units of fusion protein BTC activity were localized per 1×10⁶ (1.2 mg) SKW cells.

In patients treated with 200 mg/m2 CPT-11, plasma concentrations of CPT-11 remained above 0.1 μM for at least 24 hours (Ducreux et al., Ann Oncol 14 Suppl 2: ii17-23 (2003)). CPT-11 is dosed as a single agent at up to 500 mg/m² and plasma peak concentration is dose-proportional (Mathijssen et al. Clin Cancer Res 7: 2182-94 (2001); Ducreux et al., supra, 2003). These findings demonstrate the targeted killing of tumor cells with anti-CD20-BChE.4-1 in vitro at concentrations of CPT-11 that are pharmacologically relevant and sustainable over time in patients.

### EXAMPLE VIII

### Optimization of Butyrylcholinesterase for the Improved Activation of CPT-11

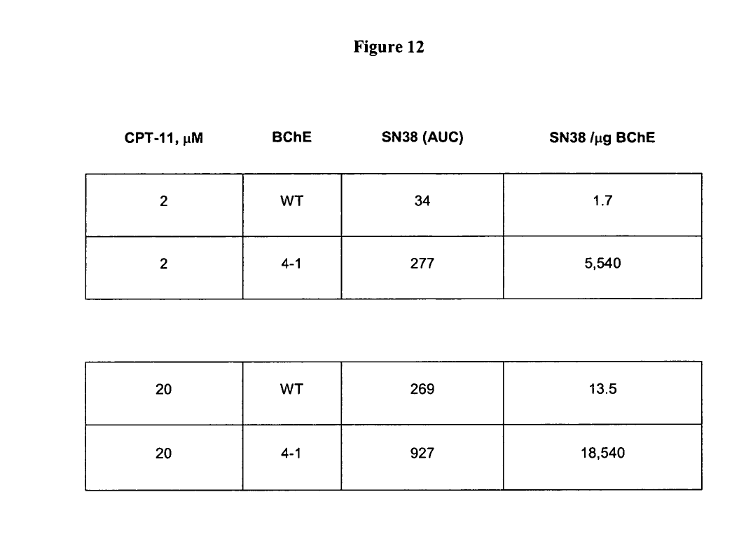

This example demonstrates identifation of additive variants incorporating beneficial mutations from different library regions that resulted, inter alia, in the isolation of the H77F, F227A, P285N, V331A variant (SEQ ID NO: 180), also referred to as the 4-1 variant, exhibiting a >3000-fold increase in CPT-11 hydrolysis over wild-type BChE (FIG. 12) and a 5-6-fold improvement in the Km for CPT-11, from 40 μM to ˜7 μM.

A series of focused BChE variant libraries corresponding to amino acids predicted to be lining the active site gorge (Harel et al., supra, 1992) of the enzyme was synthesized. Using pools of oligonucleotides encoding single amino acid mutations for 7 different library regions of 6-15 residues, libraries were synthesized using uracil-containing single-stranded DNA as a template for oligonucleotide-directed mutagenesis (Glaser et al., supra, 1992). Transformed libraries were plated on agar, and bacterial clones were picked and grown in a 96-well format for DNA plasmid preps.

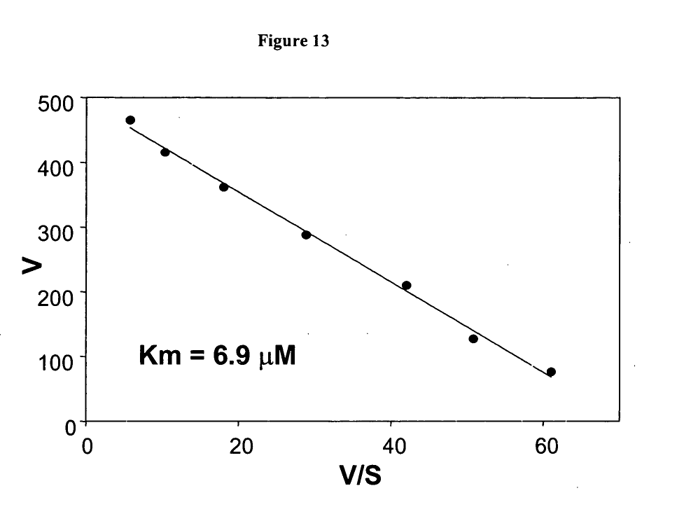

BChE plasmid variants were expressed transiently in the human embryonic kidney cell line 293T and assayed for activity at 72 hours post-transfection. Primary screening of variants was performed by indirect measurement of enzymatic activation of CPT-11 (FIG. 2) in SW48 colon carcinoma cell cytotoxicity assays. Enzyme expression in this system varies less than 20% between wells. To avoid identification of expression variants, only hits exhibiting >2-fold increased cytotoxicity were picked. Functional hits identified in these assays were characterized by sequence analysis of the corresponding DNA from the original preps. Primary screening resulted in the identification of 65 beneficial amino acid changes at 25 different positions in the enzyme. Direct measurement of BChE-mediated CPT-11 hydrolysis was quantitated by fluorescence detection of SN38 separated by reverse-phase HPLC (Dodds and Rivory, Mol Pharmacol 56: 1346-53 (1999). This method was used to compare SN38 formation by expressed variants to that of the wild-type enzyme and to calculate the Km of variants for CPT-11. Identification of additive variants incorporating beneficial mutations from different library regions resulted in the isolation of a variant exhibiting a >3000-fold increase in CPT-11 hydrolysis over wild-type BChE (FIG. 12) and a 5-6-fold improvement in the Km for CPT-11, from 40 μM to ˜7 μM (FIG. 13). FIG. 13 shows a Hofstee plot of CPT-11 hydrolysis by BChE variant 4-1. Enzyme was incubated at 37° C. for 24 hours with CPT-11 over a range of concentrations from 1-80 μM. The variant exhibits a Km of 6.9 μM, a >5-fold improvement over the 40 μM Km characteristic of native serum BChE. Velocity (v) is in light absorbance units at 540 nm. Substrate concentration [s] is μM CPT-11.

For the quantitation of SN38 formation by BChE variant 4-1, wild type (20 μg) or BChE.4-1 (0.05 μg) enzymes were incubated with CPT-11 at 37° C. for 24 hours. Samples were acidified and resolved by reverse-phase HPLC on a NovaPak C18 column to separate SN38 from unactivated CPT-11. SN38 peak area under the curve (AUC) was used to compare CPT-11 activation by the wild-type enzyme to the optimized BChE.4-1 variant.

Throughout this application various publications have been referenced within parentheses. The disclosures of each of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A butyrylcholinesterase variant comprising the amino acid sequence selected from the group consisting of SEQ ID NOS: 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof.

2. The butyrylcholinesterase variant of claim 1, having at least a two-fold increase in camptothecin conversion activity compared to butyrylcholinesterase, or functional fragment thereof.

3. A butyrylcholinesterase variant comprising the amino acid sequence selected from the group consisting of SEQ ID NOS: 24, 26, 30, 32, 34, 36, 38, 104, 106, 108, 110, 112, 116, 118, 120, 122, 124, 126, 128, 132, 134, 136, 140, and 142, or functional fragment thereof.

4. The butyrylcholinesterase variant of claim 3, having at least a fifty-fold increase in camptothecin conversion activity compared to butyrylcholinesterase, or functional fragment thereof.

5. A butyrylcholinesterase variant comprising the amino acid sequence selected from the group consisting of SEQ ID NOS: 36, 108, 110, 112, 122, 124, 134, 178, 180, 182, 186, 188, 190, 192 and 196, or functional fragment thereof.

6. The butyrylcholinesterase variant of claim 5, having at least a one hundred-fold increase in camptothecin conversion activity compared to butyrylcholinesterase, or functional fragment thereof.

7. A butyrylcholinesterase variant comprising the amino acid sequence selected from the group consisting of SEQ ID NOS: 178, 180, 182, 184, 186, 188, 192 and 196, or functional fragment thereof.

8. The butyrylcholinesterase variant of claim 7, having at least a five hundred-fold increase in camptothecin conversion activity compared to butyrylcholinesterase, or functional fragment thereof.

9. A butyrylcholinesterase variant comprising the amino acid sequence selected from the group consisting of SEQ ID NOS: 178, 180, 182, 184, 188 and 192, or functional fragment thereof.

10. The butyrylcholinesterase variant of claim 9, having at least a six hundred-fold increase in camptothecin conversion activity compared to butyrylcholinesterase, or functional fragment thereof.

11. A butyrylcholinesterase variant comprising the amino acid sequence selected from the group consisting of SEQ ID NOS: 178, 180, 182, 184 and 188, or functional fragment thereof, or functional fragment thereof.

12. The butyrylcholinesterase variant of claim 11, having at least an eight hundred hundred-fold increase in camptothecin conversion activity compared to butyrylcholinesterase, or functional fragment thereof.

13. A butyrylcholinesterase variant comprising the amino acid sequence selected from the group consisting of SEQ ID NOS: 178, 180, 184 and 188, or functional fragment thereof.

14. The butyrylcholinesterase variant of claim 13, having at least a fifteen hundred-fold increase in camptothecin conversion activity compared to butyrylcholinesterase, or functional fragment thereof.

15. A butyrylcholinesterase variant comprising the amino acid sequence selected from the group consisting of SEQ ID NOS: 178, 180 and 188, or functional fragment thereof.

16. The butyrylcholinesterase variant of claim 15, having at least a two thousand-fold increase in camptothecin conversion activity compared to butyrylcholinesterase, or functional fragment thereof.

17. A butyrylcholinesterase variant comprising the amino acid sequence selected from the group consisting of SEQ ID NOS: 178 and 180, or functional fragment thereof.

18. The butyrylcholinesterase variant of claim 17, having at least a two thousand five hundred-fold increase in camptothecin conversion activity compared to butyrylcholinesterase, or functional fragment thereof.

19. A butyrylcholinesterase variant comprising the amino acid sequence designated SEQ ID NO: 180, or functional fragment thereof.

20. The butyrylcholinesterase variant of claim 19, having at least a three thousand-fold increase in camptothecin conversion activity compared to butyrylcholinesterase, or functional fragment thereof.

21. The butyrylcholinesterase variant of claim 1, 3, 5, 7, 9, 11, 13, 15, 17 or 19, or functional fragment thereof, further comprising an antibody or antibody fragment.

22. The butyrylcholinesterase variant of claim 21, wherein said antibody or antibody fragment specifically binds the epidermal growth factor receptor (EGFR).

23. The butyrylcholinesterase variant of claim 22, wherein said antibody or antibody fragment comprises an amino acid sequence as shown in SEQ ID NOS: 18 and 20.

24. The butyrylcholinesterase variant of claim 21, wherein said antibody or antibody fragment specifically binds the CD20 cell surface antigen.

25. The butyrylcholinesterase variant of claim 24, wherein said antibody or antibody fragment comprises an amino acid sequence as shown in SEQ ID NOS: 198 and 200.

26. The butyrylcholinesterase variant of claim 25, comprising the sequence shown in FIG. 19 and designated SEQ ID NO: 202.

27. The butyrylcholinesterase variant of claim 7, wherein said amino acid sequence comprises SEQ ID NO: 178.

28. The butyrylcholinesterase variant of claim 7, wherein said amino acid sequence comprises SEQ ID NO: 180.

29. The butyrylcholinesterase variant of claim 7, wherein said amino acid sequence comprises SEQ ID NO: 182.

30. The butyrylcholinesterase variant of claim 7, wherein said amino acid sequence comprises SEQ ID NO: 184.

31. The butyrylcholinesterase variant of claim 7, wherein said amino acid sequence comprises SEQ ID NO: 186.

32. The butyrylcholinesterase variant of claim 7, wherein said amino acid sequence comprises SEQ ID NO: 188.

33. The butyrylcholinesterase variant of claim 5, wherein said amino acid sequence comprises SEQ ID NO: 190.

34. The butyrylcholinesterase variant of claim 7, wherein said amino acid sequence comprises SEQ ID NO: 192.

35. The butyrylcholinesterase variant of claim 7, wherein said amino acid sequence comprises SEQ ID NO: 194.

36. The butyrylcholinesterase variant of claim 7, wherein said amino acid sequence comprises SEQ ID NO: 196.

37. A nucleic acid encoding a butyrylcholinesterase variant comprising the nucleic acid sequence selected from SEQ ID NOS: 3, 5, 7, 9, 11, 13, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, and 195, or fragment thereof.

38. The nucleic acid of claim 37, wherein said nucleic acid sequence comprises SEQ ID NO: 177, or a functional fragment thereof.

39. The nucleic acid of claim 37, wherein said nucleic acid sequence comprises SEQ ID NO: 179, or a functional fragment thereof.

40. The nucleic acid of claim 37, wherein said nucleic acid sequence comprises SEQ ID NO: 181, or a functional fragment thereof.

41. The nucleic acid of claim 37, wherein said nucleic acid sequence comprises SEQ ID NO: 183, or a functional fragment thereof.

42. The nucleic acid of claim 37, wherein said nucleic acid sequence comprises SEQ ID NO: 185, or a functional fragment thereof.

43. The nucleic acid of claim 37, wherein said nucleic acid sequence comprises SEQ ID NO: 187, or a functional fragment thereof.

44. The nucleic acid of claim 37, wherein said nucleic acid sequence comprises SEQ ID NO: 189, or a functional fragment thereof.

45. The nucleic acid of claim 37, wherein said nucleic acid sequence comprises SEQ ID NO: 191, or a functional fragment thereof.

46. The nucleic acid of claim 37, wherein said nucleic acid sequence comprises SEQ ID NO: 193, or a functional fragment thereof.

47. The nucleic acid of claim 37, wherein said nucleic acid sequence comprises SEQ ID NO: 195, or a functional fragment thereof.

48. A method of converting a camptothecin derivative to a topoisomerase inhibitor comprising contacting said camptothecin derivative with a butyrylcholinesterase variant comprising an amino acid sequence selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194 and 196, or functional fragment thereof, under conditions that allow conversion of a camptothecin derivative to a topoisomerase inhibitor.

49. The method of claim 48, wherein said butyrylcholinesterase variant exhibits a two-fold or greater increase in conversion capability compared to butyrylcholinesterase.

50. A method of converting a camptothecin derivative to a topoisomerase inhibitor comprising contacting said camptothecin derivative with a butyrylcholinesterase variant comprising an amino acid sequence selected from SEQ ID NOS: 24, 26, 30, 32, 34, 36, 38, 104, 106, 108, 110, 112, 116, 118, 120, 122, 124, 126, 128, 132, 134, 136, 140 and 142, or functional fragment thereof, under conditions that allow conversion of a camptothecin derivative to a topoisomerase inhibitor.

51. The method of claim 50, wherein said butyrylcholinesterase variant exhibits a fifty-fold or greater increase in conversion capability compared to butyrylcholinesterase.

52. A method of converting a camptothecin derivative to a topoisomerase inhibitor comprising contacting said camptothecin derivative with a butyrylcholinesterase variant comprising an amino acid sequence selected from the group consisting of SEQ ID NOS: 36, 108, 110, 112, 122, 124, 134, 178, 180, 182, 186, 188, 190, 192 and 196, or functional fragment thereof, under conditions that allow conversion of a camptothecin derivative to a topoisomerase inhibitor.

53. The method of claim 52, wherein said butyrylcholinesterase variant exhibits a one hundred-fold or greater increase in conversion capability compared to butyrylcholinesterase.

54. A method of converting a camptothecin derivative to a topoisomerase inhibitor comprising contacting said camptothecin derivative with a butyrylcholinesterase variant comprising an amino acid sequence selected from the group consisting of SEQ ID NOS: 178, 180, 182, 184, 186, 188, 192 and 196, or functional fragment thereof, under conditions that allow conversion of a camptothecin derivative to a topoisomerase inhibitor.

55. The method of claim 54, wherein said butyrylcholinesterase variant exhibits a five hundred-fold or greater increase in conversion capability compared to butyrylcholinesterase.

56. A method of converting a camptothecin derivative to a topoisomerase inhibitor comprising contacting said camptothecin derivative with a butyrylcholinesterase variant comprising an amino acid sequence selected from the group consisting of SEQ ID NO: 178, 180, 182, 184, 188 and 192, or functional fragment thereof, under conditions that allow conversion of a camptothecin derivative to a topoisomerase inhibitor.

57. The method of claim 56, wherein said butyrylcholinesterase variant exhibits a six hundred-fold or greater increase in conversion capability compared to butyrylcholinesterase.

58. A method of converting a camptothecin derivative to a topoisomerase inhibitor comprising contacting said camptothecin derivative with a butyrylcholinesterase variant comprising an amino acid sequence selected from the group consisting of SEQ ID NO: 178, 180, 182, 184 and 188, or functional fragment thereof, under conditions that allow conversion of a camptothecin derivative to a topoisomerase inhibitor.

59. The method of claim 58, wherein said butyrylcholinesterase variant exhibits a eight hundred-fold or greater increase in conversion capability compared to butyrylcholinesterase.

60. A method of converting a camptothecin derivative to a topoisomerase inhibitor comprising contacting said camptothecin derivative with a butyrylcholinesterase variant comprising an amino acid sequence selected from the group consisting of SEQ ID NO: 178, 180, 184 and 188, or functional fragment thereof, under conditions that allow conversion of a camptothecin derivative to a topoisomerase inhibitor.

61. The method of claim 60, wherein said butyrylcholinesterase variant exhibits a fifteen hundred-fold or greater increase in conversion capability compared to butyrylcholinesterase.

62. A method of converting a camptothecin derivative to a topoisomerase inhibitor comprising contacting said camptothecin derivative with a butyrylcholinesterase variant comprising an amino acid sequence selected from the group consisting of SEQ ID NO: 178, 180 and 188, or functional fragment thereof, under conditions that allow conversion of a camptothecin derivative to a topoisomerase inhibitor.

63. The method of claim 62, wherein said butyrylcholinesterase variant exhibits a two thousand-fold or greater increase in conversion capability compared to butyrylcholinesterase.

64. A method of converting a camptothecin derivative to a topoisomerase inhibitor comprising contacting said camptothecin derivative with a butyrylcholinesterase variant comprising an amino acid sequence selected from the group consisting of SEQ ID NO: 178 and 180, or functional fragment thereof, under conditions that allow conversion of a camptothecin derivative to a topoisomerase inhibitor.

65. The method of claim 64, wherein said butyrylcholinesterase variant exhibits a two thousand five hundred-fold or greater increase in conversion capability compared to butyrylcholinesterase.

66. A method of converting a camptothecin derivative to a topoisomerase inhibitor comprising contacting said camptothecin derivative with a butyrylcholinesterase variant comprising an amino acid sequence designated SEQ ID NO: 180, or functional fragment thereof, under conditions that allow conversion of a camptothecin derivative to a topoisomerase inhibitor.

67. The method of claim 66, wherein said butyrylcholinesterase variant exhibits a three thousand-fold or greater increase in conversion capability compared to butyrylcholinesterase.

68. The method of claim 48, 50, 52, 54, 56, 58, 60, 62, 64 or 66, wherein said topoisomerase inhibitor is SN-38.

69. The method of claim 68, wherein said camptothecin derivative is CPT-11.

70. The method of claim 54, wherein said butyrylcholinesterase variant comprises the amino acid sequence shown in SEQ ID NO: 178, or a functional fragment thereof.

71. The method of claim 54, wherein said butyrylcholinesterase variant comprises the amino acid sequence shown in SEQ ID NO: 180, or a functional fragment thereof.

72. The method of claim 54, wherein said butyrylcholinesterase variant comprises the amino acid sequence shown in SEQ ID NO: 182, or a functional fragment thereof.

73. The method of claim 54, wherein said butyrylcholinesterase variant comprises the amino acid sequence shown in SEQ ID NO: 184, or a functional fragment thereof.

74. The method of claim 54, wherein said butyrylcholinesterase variant comprises the amino acid sequence shown in SEQ ID NO: 186, or a functional fragment thereof.

75. The method of claim 54, wherein said butyrylcholinesterase variant comprises the amino acid sequence shown in SEQ ID NO: 188, or a functional fragment thereof.

76. The method of claim 52, wherein said butyrylcholinesterase variant comprises the amino acid sequence shown in SEQ ID NO: 190, or a functional fragment thereof.

77. The method of claim 54, wherein said butyrylcholinesterase variant comprises the amino acid sequence shown in SEQ ID NO: 192, or a functional fragment thereof.

78. The method of claim 54, wherein said butyrylcholinesterase variant comprises the amino acid sequence shown in SEQ ID NO: 194, or a functional fragment thereof.

79. The method of claim 54, wherein said butyrylcholinesterase variant comprises the amino acid sequence shown in SEQ ID NO: 196, or a functional fragment thereof.

80. A method of treating cancer comprising administering to an individual an effective amount of a butyrylcholinesterase variant selected from SEQ ID NOS: 2, 4, 6, 8, 10, 12, 14, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, and 196, or functional fragment thereof, exhibiting increased capability to convert a camptothecin derivative to a topoisomerase inhibitor compared to butyrylcholinesterase.

81. The method of claim 80, wherein said cancer is metastatic colorectal cancer.

82. The method of claim 80, wherein said cancer is ovarian cancer.

83. The method of claim 80, wherein said cancer is lung cancer.

84. The method of claim 80, wherein said cancer is non-Hodgkin's lymphoma.

85. The method of claim 80, wherein said topoisomerase inhibitor is SN-38.

86. The method of claim 80, wherein said camptothecin derivative is CPT-11.

87. The method of claim 80, wherein said butyrylcholinesterase variant comprises the amino acid sequence shown in SEQ ID NO: 178, or a functional fragment thereof.

88. The method of claim 80, wherein said butyrylcholinesterase variant comprises the amino acid sequence shown in SEQ ID NO: 180, or a functional fragment thereof.

89. The method of claim 80, wherein said butyrylcholinesterase variant comprises the amino acid sequence shown in SEQ ID NO: 182, or a functional fragment thereof.

90. The method of claim 80, wherein said butyrylcholinesterase variant comprises the amino acid sequence shown in SEQ ID NO: 184, or a functional fragment thereof.

91. The method of claim 80, wherein said butyrylcholinesterase variant comprises the amino acid sequence shown in SEQ ID NO: 186, or a functional fragment thereof.

92. The method of claim 80, wherein said butyrylcholinesterase variant comprises the amino acid sequence shown in SEQ ID NO: 188, or a functional fragment thereof.

93. The method of claim 80, wherein said butyrylcholinesterase variant comprises the amino acid sequence shown in SEQ ID NO: 190, or a functional fragment thereof.

94. The method of claim 80, wherein said butyrylcholinesterase variant comprises the amino acid sequence shown in SEQ ID NO: 192, or a functional fragment thereof.

95. The method of claim 80, wherein said butyrylcholinesterase variant comprises the amino acid sequence shown in SEQ ID NO: 194, or a functional fragment thereof.

96. The method of claim 80, wherein said butyrylcholinesterase variant comprises the amino acid sequence shown in SEQ ID NO: 196, or a functional fragment thereof.

97. The method of claim 80, wherein said butyrylcholinesterase variant further comprises an antibody or antibody fragment.

98. The method of claim 97, wherein said antibody or antibody fragment specifically binds the epidermal growth factor receptor (EGFR).

99. The method of claim 98, wherein said antibody or antibody fragment comprises an amino acid sequence as shown in SEQ ID NOS: 18 and 20.

100. The method of claim 97, wherein said antibody or antibody fragment specifically binds the CD20 cell surface antigen.

101. The method of claim 100, wherein said antibody or antibody fragment comprises an amino acid sequence as shown in SEQ ID NOS: 198 and 200.

102. The method of claim 97, wherein said butyrylcholinesterase comprises the sequence shown in FIG. 19 and designated SEQ ID NO: 202.

103. The method of claim 97, wherein said butyrylcholinesterase variant comprises the amino acid sequence designated as SEQ ID NO: 180, or functional fragment thereof.

104. The method of claim 103, wherein said functional fragment is a L530 truncation (SEQ ID NO.: 204).
